(19) United States
(12) Patent Application Publication
(10) Pub. No.: US 2022/0296586 A1
(43) Pub. Date: Sep. 22, 2022

(54) NOVEL COMPOSITIONS, COMBINATIONS, AND METHODS THEREOF

(71) Applicants: Sreenivasarao Vepachedu, VERNON HILLS, IL (US); EXCIVA GmbH, HEIDELBERG (DE)

(72) Inventors: Sreenivasarao Vepachedu, VERNON HILLS, IL (US); Hans J. Moebius, Wolleru (DE); Anton Bespalov, Heidelberg (DE)

(73) Assignee: Sreenivasarao Vepachedu, VERNON HILLS, IL (US)

(21) Appl. No.: 17/827,958

(22) Filed: May 30, 2022

Related U.S. Application Data
(63) Continuation-in-part of application No. 16/712,010, filed on Dec. 12, 2019.
Application No. 16/712,010 is a continuation of application No. 16/672,517, filed on Nov. 3, 2019.
Application No. 16/672,517 is a continuation-in-part of application No. 16/328,678, filed on Feb. 26, 2019, now Pat. No. 11,103,499.
(60) Provisional application No. 62/501,696, filed on May 4, 2017.
(60) Provisional application No. 62/477,435, filed on Mar. 28, 2017.
(60) Provisional application No. 62/380,325, filed on Aug. 26, 2016.

(51) Int. Cl. A61K 31/4748; A61K 31/662; A61K 31/222; A61K 31/225

(52) U.S. Cl. CPC A61K 31/4748; A61K 31/662; A61K 31/222; A61K 31/225

(57) ABSTRACT

Compounds of Formula I:

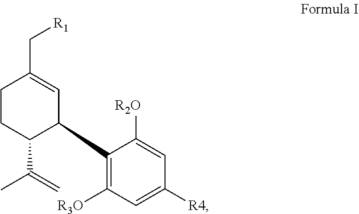

pharmaceutically acceptable salts thereof, enantiomers thereof, metabolites thereof, derivatives thereof, prodrugs thereof, acid addition salts thereof, pharmaceutically acceptable salts thereof, or N-oxides thereof; or a combination thereof, processes and intermediates for preparation thereof, compositions thereof, and uses thereof, are provided. Pharmaceutical compositions comprising a compound of Formula I, or enantiomers thereof, metabolites thereof, derivatives thereof, prodrugs thereof, acid addition salts thereof, pharmaceutically acceptable salts thereof, or N-oxides thereof, or a combination thereof, wherein the compound is double and/or triple agent or ligand for CYP2D6, 5-HT_{2A} receptors, and/or acetylcholinesterase; and methods comprising co-administering a compound of Formula I and an N-Methyl-d-Aspartate (NMDA) receptor antagonist to a subject in need thereof, and dosage forms, drug delivery systems, methods of treatment thereof. The compositions contain one of more ligands for CYP2D6, 5-HT_{2A}, and N-Methyl-d-Aspartate (NMDA) receptors, and acetylcholinesterase.

### TECHNICAL FIELD

This disclosure relates to novel compositions, combinations, therapeutic formulations, symptomatic and disease-modifying treatments, therapies, kits thereof, and methods thereof.

### BACKGROUND

Diseases affecting the brain and central nervous system represent one of the largest global healthcare challenges and greatest medical needs due to the devastating personal and economic consequences for patients, caregivers and society. An estimated 55 million people worldwide suffer from neurodegenerative diseases with no currently approved disease-modifying therapies available. As modern therapeutic interventions increase life expectancy, the number of patients suffering from these diseases is expected to double every 20 years. Just nine of the most common neurological diseases such as Alzheimer's disease and other dementias, low back pain, stroke, traumatic brain injury, migraine, epilepsy, multiple sclerosis, spinal cord injury, and Parkinson's disease is staggering, totaling $789 billion in 2014 dollars, currently estimated at $818 billion, and estimated to be more than $1 trillion by 2030. Total European 2010 cost of brain disorders was €798 billion, of which direct health care cost 37%, direct non-medical cost 23%, and indirect cost 40%.

Progress in specifically addressing therapeutic needs in dementia has been slow in the past two decades, and all development projects in Alzheimer's Disease have failed since the approval of memantine by the EMA (2002) and the FDA (2003). Rather than insisting on treatment indications, regulators have addressed the persisting high medical need by opening up the range of approvable medications to therapies with syndromal indication labels. Such a syndromal indication label could cover, e.g., Behavioral and Psychiatric Symptoms in Dementia (BPSD), sub-syndromal indications like aggression or apathy in Alzheimer's Disease, hallucinations and delusions in Parkinson's Disease Dementia (PDD). BPSD also known as neuropsychiatric symptoms, represent a heterogeneous group of non-cognitive symptoms and behaviors occurring in subjects with dementia. BPSD constitute a major component of the dementia syndrome irrespective of its subtype. They are as clinically relevant as cognitive symptoms as they strongly correlate with the degree of functional and cognitive impairment. BPSD include agitation, aberrant motor behavior, anxiety, elation, irritability, depression, apathy, disinhibition, delusions, hallucinations, and sleep or appetite changes. It is estimated that BPSD affect up to 90% of all dementia subjects over the course of their illness, and is independently associated with poor outcomes, including distress among patients and caregivers, long term hospitalization, misuse of medication and increased health care costs. Accordingly, there is need for development of such new therapies with syndromal indication labels for novel compositions, combinations, therapeutic formulations, symptomatic and disease-modifying treatments, and therapies.

### SUMMARY OF THE INVENTION

An embodiment of the invention is a composition comprising a compound of Formula I:

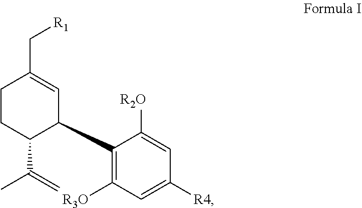

wherein R₁, R₂, R₃, and R₄ are independently H, OH, or Alkyl (C₁-C₁₀), or enantiomers thereof, metabolites thereof, derivatives thereof, and/or prodrugs thereof, pharmaceutically acceptable salts thereof, N-oxides thereof, or a combination thereof.

An embodiment of the invention is a composition comprising a compound of Formula II

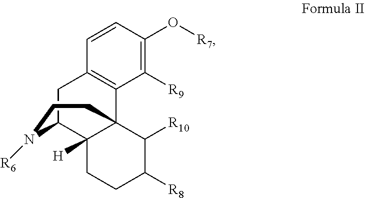

wherein, R₆, R₇, and R₈ are independently H, D, C₁₋₁₀-alkyl, halo C₁₋₁₀-alkyl wherein halogen is F, Cl, or Br; R₉ and R₁₀ are independently H; C₁₋₁₀-alkyl; halo C₁₋₁₀-alkyl wherein halogen is F, Cl, or Br; OH; or R₉ and R₁₀ together form a five-membered heterocycle wherein the hetero atom is O, S, or N; enantiomers, metabolites, derivatives, prodrugs, salts, diastereomers, pharmaceutically acceptable salts, or N-oxides thereof, or a combination thereof, or 3) a combination of 1 and 2; or a combinations thereof.

In some embodiments, the composition is one or more compounds of formula I, or formula II, or a combination thereof.

Some embodiments include a composition comprising an effective amount of:

a composition comprising a compound of Formula I, wherein the compound is

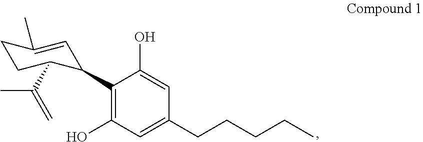

or enantiomers thereof, metabolites thereof, derivatives thereof, and/or prodrugs thereof, pharmaceutically acceptable salts thereof, N-oxides thereof, or a combination thereof; and
a compound of Formula II

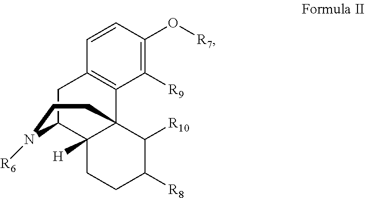

wherein, R₆, R₇, and R₈ are independently H, D, C₁₋₁₀-alkyl, halo C₁₋₁₀-alkyl wherein halogen is F, Cl, or Br; R₉ and R₁₀ are independently H; C₁₋₁₀-alkyl; halo C₁₋₁₀-alkyl wherein halogen is F, Cl, or Br; OH; or R₉ and R₁₀ together form a five-membered heterocycle wherein the hetero atom is O, S, or N; enantiomers, metabolites, derivatives, prodrugs, salts, diastereomers, pharmaceutically acceptable salts, or N-oxides thereof, or a combination thereof, or 3) a combination of 1 and 2; or a combinations thereof.

Some embodiments include a method of treating a disease or disorder in a subject in need thereof comprising an effective amount of: 1) a composition comprising a compound of Formula I, as defined above; enantiomers thereof, metabolites thereof, derivatives thereof, and/or prodrugs thereof, pharmaceutically acceptable salts thereof, N-oxides thereof, or a combination thereof, or 2) a compound of Formula II, as defined above, enantiomers, metabolites, derivatives, prodrugs, salts, diastereomers, pharmaceutically acceptable salts, or N-oxides thereof, or a combination thereof, or 3) a combination of 1 and 2.

Some embodiments include a method of treating a disease or disorder in a subject in need thereof comprising an effective amount of a composition comprising dextromethorphan, enantiomers, metabolites, derivatives, or prodrugs thereof, or a combination thereof; salts and diastereomers thereof, pharmaceutically acceptable salts thereof, N-oxides thereof, processes and intermediates for preparation thereof, compositions thereof, and uses thereof.

In an embodiment, the method is a method of decreasing the number of doses and/or total daily dose of the compound of Formula II that can be administered while increasing efficacy and safeguarding tolerability and safety; a method of reducing an adverse event associated with treatment by the compound of Formula II, wherein the subject is at risk of experiencing the adverse event as a result being treated with the compound of Formula II; a method of decreasing metabolites of the compound of Formula II plasma levels, a method of treating a neurological disorder, a method of increasing the compound of Formula II plasma levels in a subject in need of treatment with the compound of Formula II, wherein the subject is an extensive metabolizer of the compound of Formula II; a method of inhibiting the metabolism of the compound of Formula II; a method of increasing the metabolic lifetime of the compound of Formula II; a method of correcting extensive metabolism of the compound of Formula II; a method of improving the antitussive properties of the compound of Formula II; a method of treating cough. Another embodiment is the method, wherein the disease or disorder is a neurological disorder, wherein the composition is administered at least once a day for at least 1 day, 2 days, 3 days, 4 days, 5 days, 6 days, 7 days, or 8 days.

Some embodiments include a method of treating a neurological disorder comprising administering about 5 mg/day to about 600 mg/day, about 5 mg/day to about 300 mg/day, about 5 mg/day to about 400 mg/day, about 5 mg/day to about 500 mg/day, about 5 mg/day to about 600 mg/day, about 5 mg/day to about 1,000 mg/day, about 50 mg/day to about 1000 mg/day, about 100 mg/day to about 1000 mg/day, about 150 mg/day to about 1000 mg/day, about 150 mg/day to about 5000 mg/day, about 150 mg/day to about 300 mg/day, or about 150 mg/day to about 100 mg/day, or an amount as required of a compound of Formula I, and about 0.1 mg/day to about 1 mg/day, about 0.5 mg/day to about 15 mg/day, about 15 mg/day to about 60 mg/day, about 15 mg/day to about 120 mg/day, about 0.1 mg/day to about 200 mg/day, or an amount as required of the compound of Formula II to a subject in need thereof.

Another embodiment is a pharmaceutical composition comprising the compound of Formula II and one or more agents are compounds of Formula I.

### BRIEF DESCRIPTION OF FIGURES

FIG. 1. Activity of rats in the open field under moderately aversive conditions. Rats were pre-treated with a combination of dextromethorphan (or its vehicle) and cannabidiol (or its vehicle) prior to being placed in the open field for 60 min. Data are presented as mean (±SEM) activity recorded as arbitrary units of crossing the open field zones. N=8 per treatment condition.

FIG. 2. Anxety index of rats in the open field under moderately aversive conditions. Rats were pre-treated with a combination of dextromethorphan (or its vehicle) and cannabidiol (or its vehicle) prior to being placed in the open field for 60 min. Data are presented as mean (±SEM) anxiety index calculated as the relative proximity to the novel object placed in one of the open field corners. N=7-8 per treatment condition.

FIG. 3: Lineweaver-Burk plots for the inhibition of CYP2D6 by cannabidiol (CBD). Recombinant CYP2D6 was incubated with dextromethorphan in the presence or absence of different concentrations of CBD. Each point is the mean of duplicate determinations.

### DETAILED DESCRIPTION OF THE INVENTION

Various embodiments comprise novel compositions, and combinations, therapeutic formulations, symptomatic and disease-modifying treatments, therapies, kits thereof, and methods of making such compositions, combinations, therapeutic formulations, treatments, therapies, and kits comprising biologics, chemicals, nutritionals, pharmaceuticals, compositions, treatments, therapies, cures, prophylactics, supplements, and formulations, including the disclosures of patent applications U.S. 62/501,693 filed May 4, 2017, PCT/US2017/048748 filed Aug. 25, 2017 published WO 2018/039642 A1 Mar. 1, 2018, TW 106129169 filed Aug. 28, 2017, U.S. 62/634,162 filed Feb. 22, 2018, U.S. 62/636,171 filed Feb. 22, 2018, U.S. 62/635,554 filed Feb. 27, 2018, and U.S. 62/636,099 filed Feb. 27, 2018, all of which are incorporated by reference.

An embodiment of the invention is a composition comprising a compound of formula I:

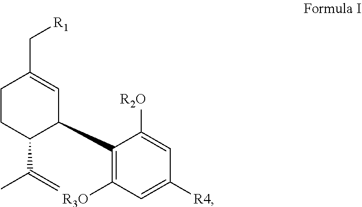

wherein R₁, R₂, R₃, and R₄ are independently H, OH, or Alkyl (C₁₋₁₀), or pharmaceutically acceptable salts, N-oxides thereof, prodrugs thereof, enantiomers thereof, metabolites thereof, derivatives thereof, or a combination thereof; and
a compound of Formula II,

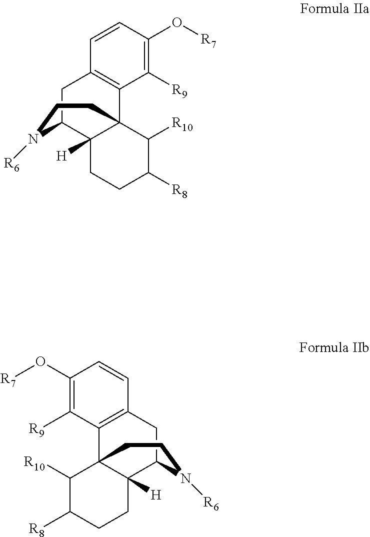

wherein R₆, R₇, and R₈ are independently H, D, C₁₋₁₀-alkyl, halo C₁₋₁₀-alkyl wherein halogen is F, Cl, or Br; R₉ and R₁₀ are independently H; C₁₋₁₀-alkyl; halo C₁₋₁₀-alkyl wherein halogen is F, Cl, or Br; OH; or R₉ and R₁₀ together form a five-membered heterocycle wherein the hetero atom is O, S, or N. In certain embodiments, the compound is a compound of Formula I wherein R₁ is an aryl, and R₂ is substituted or unsubstituted —C₁₋₁₀ alkyl-X—(Y)ₙ. In certain embodiments, R₆ is substituted or unsubstituted C₁₋₁₀ alkyl-C₁₋₁₀ aryl, and R₇ is substituted or unsubstituted —C₁₋₁₀ alkyl-X—(Y). In certain embodiments, R₆ is phenyl, R₇ is —C₁₋₁₀ alkyl-N—(C₁₋₁₀ alkyl)₂. In some embodiments, the compound of Formula I is

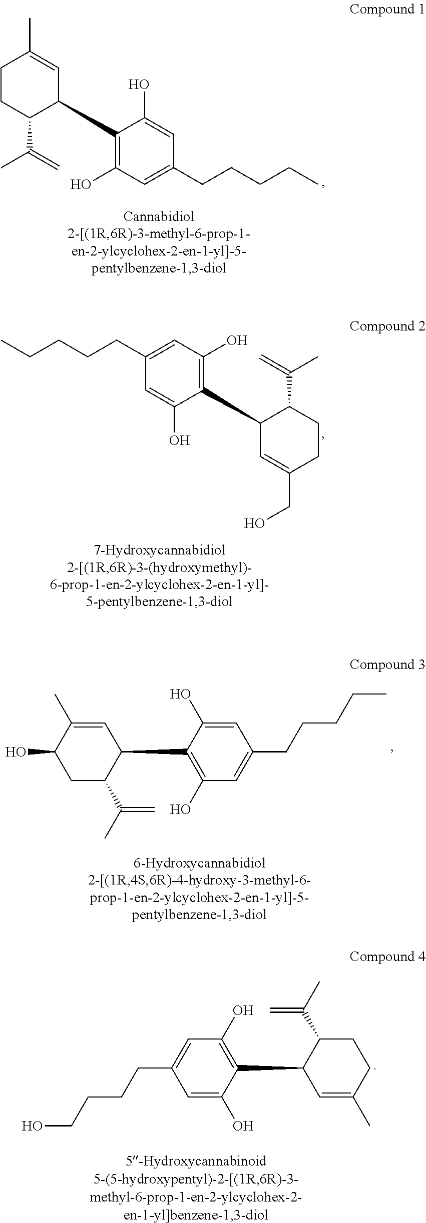

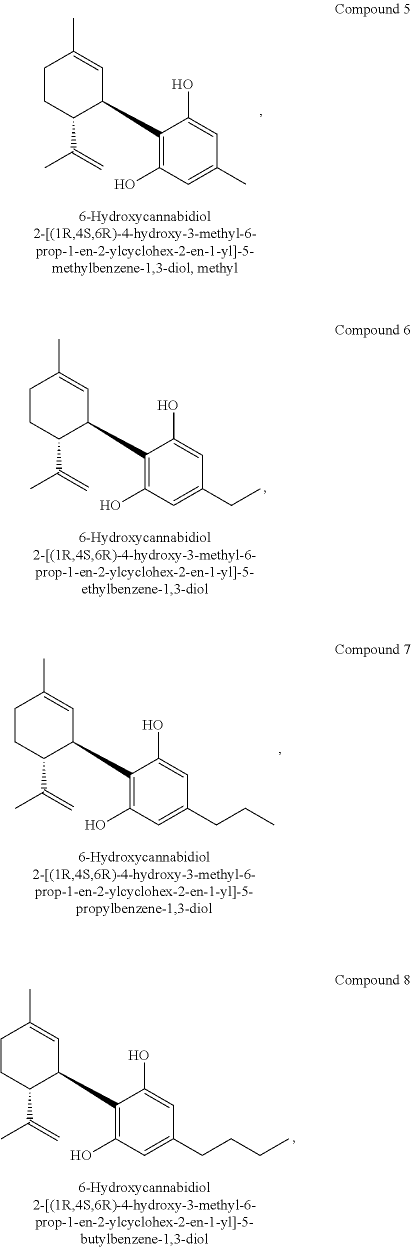 [SMILES: C=C(C)[C@@H]1CCC(C)=C[C@H]1c1c(O)cc(C)cc1O ; C=C(C)[C@@H]1CCC(C)=C[C@H]1c1c(O)cc(CC)cc1O ; C=C(C)[C@@H]1CCC(C)=C[C@H]1c1c(O)cc(CCC)cc1O ; C=C(C)[C@@H]1CCC(C)=C[C@H]1c1c(O)cc(CCCC)cc1O]

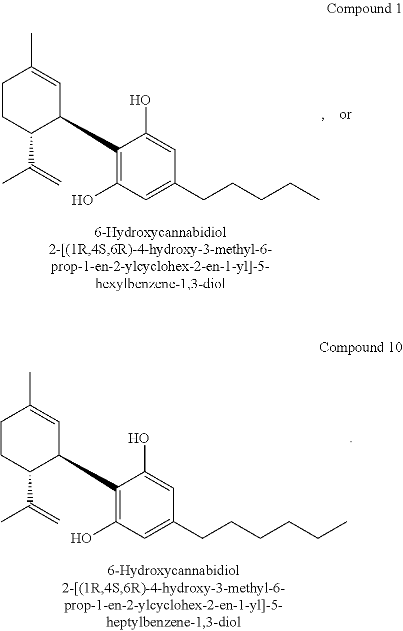 [SMILES: C=C(C)[C@@H]1CCC(C)=C[C@H]1c1c(O)cc(CCCCC)cc1O ; C=C(C)[C@@H]1CCC(C)=C[C@H]1c1c(O)cc(CCCCCC)cc1O]

In some embodiments, the composition is one or more compounds of formula I, or formula II, or a combination thereof.

In some embodiments, the compound of Formula II is a fluoro-derivative such as, but not limited to: (4bS,8aS,9S)-11-methyl-3-(trifluoromethoxy)-6,7,8,8a,9,10-hexahydro-5H-9,4b-(epiminoethano)phenanthrene; (4bS,8aS,9S)-3-(trifluoromethoxy)-11-(trifluoromethyl)-6,7,8,8a,9,10-hexahydro-5H-9,4b-(epiminoethano)phenanthrene; (4bS,8aS,9S)-3-methoxy-11-(trifluoromethyl)-6,7,8,8a,9,10-hexahydro-5H-9,4b-(epiminoethano)phenanthrene.

In some embodiments, the compound of Formula II is an acid addition salt selected from acetate, acetyl salicylate, adipate, aspartate, butyrate, caprate, caproate, caprylate, enanthate, formate, fumarate, glutamate glutarate, isophthallate, maleate, malonate, methionate, oxalate, pelargonate, pimelate, propionate, phthallate, salicylate, sebacate, succinate, terephthallate, tyrosinate, tryptophanate, valerate, N-acyl-aspartate, N-acyl-glutamate, N-acyl-tyrosinate, N-acyl-tryptophanate, N-acyl-methionate, citrate, galactonate, glucaric acid (saccharic acid), mannonate, mucate, rhamnonate, and tartrate.

In another embodiment, dextromethorphan or a compound of Formula II as defined above, and a derivative of a compound of Formula I, wherein the derivative is an acid addition salts selected from: acetate, acetyl salicylate, adipate, aspartate, butyrate, caprate, caproate, caprylate, enanthate, formate, fumarate, glutamate glutarate, isophthallate, maleate, malonate, methionate, oxalate, pelargonate, pimelate, propionate, phthallate, salicylate, sebacate, succinate, terephthallate, tyrosinate, tryptophanate, valerate, N-acyl-aspartate, N-acyl-glutamate, N-acyl-tyrosinate, N-acyl-tryptophanate, N-acyl-methionate, citrate, galactonate, glucaric acid (saccharic acid), mannonate, mucate, rhamnonate, and tartrate.

In some embodiments, the compound of Formula II is an acid addition salt selected from N-acyl-aspartate, N-acyl-glutarate, N-acyl-tyrosinate, N-acyl-tryptophanate, and N-acyl-methionate.

Examples include addition salts of base of formula II represented by dextromethorphan, such as dextromethorphan hydrogen acetate, dextromethorphan hydrogen acetyl salicylate, dextromethorphan hydrogen adipate, dextromethorphan hydrogen aspartate, dextromethorphan hydrogen butyrate, dextromethorphan hydrogen caprate, dextromethorphan hydrogen caproate, dextromethorphan hydrogen caprylate, dextromethorphan hydrogen enanthate, dextromethorphan hydrogen formate, dextromethorphan hydrogen fumarate, dextromethorphan hydrogen glutarate, dextromethorphan hydrogen isophthallate, dextromethorphan hydrogen maleate, dextromethorphan hydrogen malonate, dextromethorphan hydrogen oxalate, dextromethorphan hydrogen pelargonate, dextromethorphan hydrogen pimelate, dextromethorphan hydrogen propionate, dextromethorphan hydrogen phthallate, dextromethorphan hydrogen salicylate, dextromethorphan hydrogen sebacate, dextromethorphan hydrogen succinate, dextromethorphan hydrogen terephthallate, dextromethorphan hydrogen tyrosinate, dextromethorphan hydrogen tryptophanate, and dextromethorphan hydrogen valerate.

Another embodiment of the invention is a composition comprising an acid addition salt of compound of Formula I and an acid addition salt of a compound of Formula II. Another embodiment of the invention is a composition comprising an acid addition salt of Formula I and an acid addition salt of dextromethorphan.

Another embodiment of the invention is a composition comprising an acid addition salt of a halogenated compound of Formula I and an acid addition salt of dextromethorphan.

An embodiment of the invention is an addition salt of Formula I, wherein with organic acid such as aspartic acid, benzenesulfonic acid, besylic acid, benzoic acid, bicarbonic acid, tartaric acid, bromide, camphor sulfonic acid, camsylic acid, chloride, citric acid, decanoic acid, edetate, lauryl sulfonic acid, estolic acid, ethanesulfonic acid, esylic acid, fumaric acid, gluceptic acid, gluconic acid, glutamic acid, glycolic acid, glycollylarsanilic acid, hexanoic acid, hexylresorcinol, hydroxynaphthoic acid, isethionic acid, iodide, lactic acide, galactopyranosyl-d-gluconic acid, lactobionic acid, malic acid, maleic acid, mandelic acid, methanesulfonic acid, methylbromide, methylnitric acid, methylsulfonic acid, mucic acid, napsylic acid, nitric acid, octanoic acid, oleic acid, pamoic acid, 4,4′-methylenebis(3-hydroxy-2-naphthonic acid, pantothenic acid, phosphoric acid, polygalacturonic acid, propionic acid, salicylic acid, stearic acid, succinic acid, sulfuric acid, tartaric acid, teoclic acid, 8-chloro-1,3-dimethyl-7h-purine-2,6-dione, tosylic acid, malic acid, methionic acid, phthallic acid, malonic acid, tyrosine, tryptophan, maleic acid, fumaric acid, succinic acid, glutaric acid, adipic acid, pimelic acid, sebacic acid, formic acid, acetic acid, propionic acid, butyric acid, valeric acid, caproic acid, enanthic acid, caprylic acid, pelargonic acid, capric acid, oxalic acid, isophthallic acid, terephthallic acid, salicylic acid, difluorosuccinicacid, trifluorosuccinic acid, tetrafluorosuccinic acid, difluoroglutaric acid, difluoroacetic acid, trifluoroacetic acid; and dextromethorphan; or a combination thereof.

In some aspects of the invention, the compound of Formula I is a prodrug wherein the compound is an ester or an addition formed from the following acids: 3-(nitrooxy)propanoic acid. Examples include, but are not limited to, 3-nitrooxy derivatives of Compounds 71-73, 4-nitrooxy derivatives of Compounds 74-76, and 3-nitrooxy derivatives of Compounds 74-76. In some embodiments, the compounds of Formula I form addition salts of 3-(nitrooxy)propanoic acid, 3-(nitrooxy)butanoic acid, and 4-(nitrooxy)butanoic acid. In another embodiment, the acid addition salt is of 3-(nitrooxy)propanoic acid, 3-(nitrooxy)butanoic acid, and 4-(nitrooxy)butanoic acid.

In another embodiment, the pharmaceutically acceptable acid addition salts of the compounds of the Formula I can be formed with inorganic acids such as hydrochloric acid, hydrogen bromide, sulfuric acid, phosphoric acid and nitric acid.

Other anion salts of compounds of Formulas I and II include the salts formed from the following acidic groups:

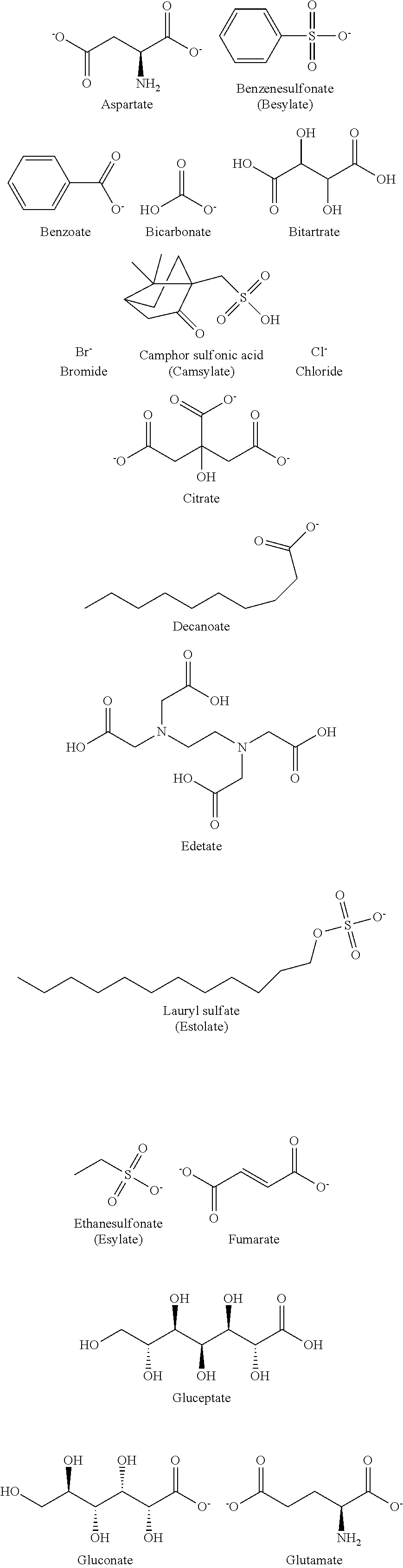

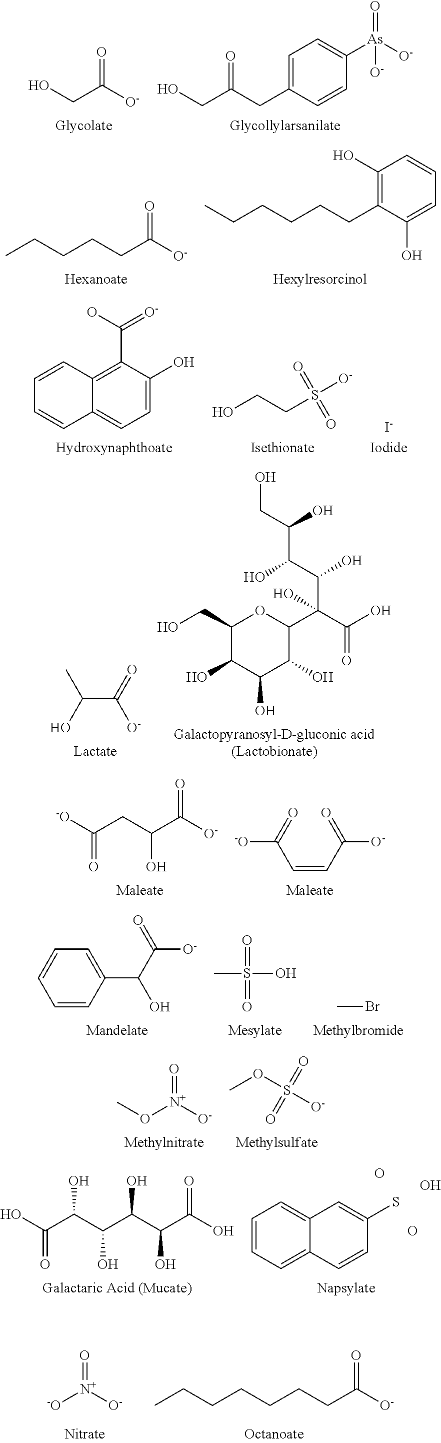

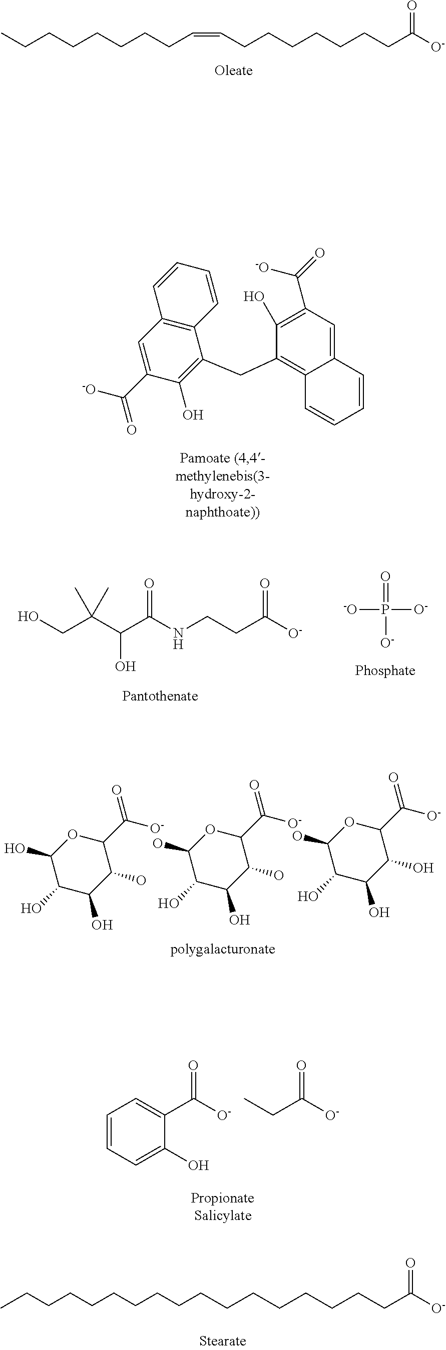

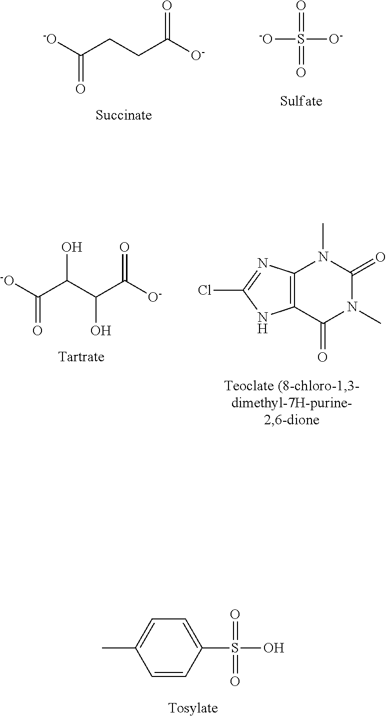

Another embodiment is a composition comprising a compound of Formula I, and at least one compound selected from thioridazine, perphenazine, fluphenazine, zuclopenthixol, risperidone, sertindole, nortriptyline, amitriptyline, imipramine, fluoxetine, paroxetine, ajmaline, amiodarone, amitriptyline, aprindine, azelastine, celecoxib, chlorpheniramine, chlorpromazine, diphenhydramine, doxorubicin, fluphenazine, fluvastatin, haloperidol, imipramine, indinavir, lasoprazole, levomepromazine, lopinavir, loratadine, mequitazine, methadone, metoclopramide, mibefradil, moclobemide, nelfinavir, nevirapine, nicardipine, norfluoxetine, perphenazine, pimozide, terfenadine, thioridazine, cimetidine, quinidine, cisapride, citalopram, clozapine, cocaine, desipramine, ranitidine, risperidone, ritonavir, saquinavir, sertraline, terbinafine, ticlopidine, trifluperidol, yohimbine, clomipramine, doxepin, mianserin, imipramine, 2-chloroimipramine, amitriptyline, amoxapine, protriptyline, trimipramine, nortriptyline, maprotiline, phenelzine, isocarboxazid, tranylcypromine, trazodone, citalopram, sertraline, aryloxy indanamine, benactyzine, escitalopram, fluvoxamine, venlafaxine, desvenlafaxine, duloxetine, mirtazapine, nefazodone, selegiline, sibutramine, milnacipran, tesofensine, brasofensine, moclobemide, rasagiline, nialamide, iproniazid, iproclozide, toloxatone, butriptyline, dosulepin, dibenzepin, iprindole, lofepramine, opipramol, and dapoxetine.

Another embodiment of the invention is a composition comprising a compound of Formula I, a compound of Formula II, and at least one compound selected from thioridazine, perphenazine, fluphenazine, zuclopenthixol, risperidone, sertindole, nortriptyline, amitriptyline, imipramine, fluoxetine, paroxetine, ajmaline, amiodarone, amitriptyline, aprindine, azelastine, celecoxib, chlorpheniramine, chlorpromazine, diphenhydramine, doxorubicin, fluphenazine, fluvastatin, haloperidol, imipramine, indinavir, lasoprazole, levomepromazine, lopinavir, loratadine, mequitazine, methadone, metoclopramide, mibefradil, moclobemide, nelfinavir, nevirapine, nicardipine, norfluoxetine, perphenazine, pimozide, terfenadine, thioridazine, cimetidine, quinidine, cisapride, citalopram, clomipramine, clozapine, cocaine, ranitidine, risperidone, ritonavir, saquinavir, sertraline, terbinafine, ticlopidine, trifluperidol, yohimbine, doxepin, mianserin, imipramine, 2-chloroimipramine, amitriptyline, amoxapine, desipramine, protriptyline, trimipramine, nortriptyline, maprotiline, phenelzine, isocarboxazid, tranylcypromine, trazodone, citalopram, sertraline, aryloxy indanamine, benactyzine, escitalopram, fluvoxamine, venlafaxine, desvenlafaxine, duloxetine, mirtazapine, nefazodone, selegiline, sibutramine, milnacipran, tesofensine, brasofensine, moclobemide, rasagiline, nialamide, iproniazid, iproclozide, toloxatone, butriptyline, dosulepin, dibenzepin, iprindole, lofepramine, opipramol, and dapoxetine.

In one embodiment, the composition comprises a compound of Formula I, and one or more of AChIs such as 2-((1-Benzylpiperidin-4-yl)methyl)-5,6-dimethoxy-2,3-dihydro-1H-inden-1-one (Donepezil), (S)-3-(1-(dimethylamino)ethyl)phenyl ethyl(methyl) carbamate (Rivastigmine), dimethyl (2,2,2-trichloro-1-hydroxyethyl)phosphonate (Metrifonate), (4aS,6R,8aS)-3-methoxy-11-methyl-4a,5,9,10,11,12-hexahydro-6H-benzo[2,3]benzofuro[4,3-cd]azepin-6-ol (Galantamine), and 1,2,3,4-tetrahydroacridin-9-amine (Tacrine), O,S-dimethyl acetylphosphoramidothioate, O,O-dimethyl S-((4-oxobenzo[d][1,2,3]triazin-3(4H)-yl)methyl) phosphorodithioate, 2,2-dimethyl-2,3-dihydrobenzofuran-7-yl methylcarbamate, S-(((4-chlorophenyl)thio)methyl) O,O-diethyl phosphorodithioate, 2-chloro-1-(2,4-dichlorophenyl)vinyl diethyl phosphate, O,O-diethyl O-(3,5,6-trichloropyridin-2-yl) phosphorothioate, O-(3-chloro-4-methyl-2-oxo-2H-chromen-7-yl) O,O-diethyl phosphorothioate, 1-phenylethyl (E)-3-((dimethoxyphosphoryl)oxy)but-2-enoate,4-(tert-butyl)-2-chlorophenyl methyl methylphosphoramidate, O,O-diethyl O-(2-(ethylthio)ethyl) phosphorothioate, O,O-diethyl S-(2-(ethylthio)ethyl) phosphorothioate, O,O-diethyl O-(2-isopropyl-6-methylpyrimidin-4-yl) phosphorothioate, 2,2-dichlorovinyl dimethyl phosphate, (E)-4-(dimethylamino)-4-oxobut-2-en-2-yl dimethyl phosphate, O,O-dimethyl S-(2-(methylamino)-2-oxoethyl) phosphorodithioate, S,S′-(1,4-dioxane-2,3-diyl) O,O,O′,O′-tetraethyl bis(phosphorodithioate), O,O-diethyl S-(2-(ethylthio)ethyl) phosphorodithioate, O-ethyl O-(4-nitrophenyl) phenylphosphonothioate, O,O,O′,O′-tetraethyl S,S′-methylene bis(phosphorodithioate), O-ethyl S,S-dipropyl phosphorodithioate, O-(4-(N,N-dimethylsulfamoyl)phenyl) O,O-dimethyl phosphorothioate, O-(4-(N,N-dimethylsulfamoyl)phenyl) O,O-dimethyl phosphorothioate, ethyl (3-methyl-4-(methylthio)phenyl) isopropylphosphoramidate, O,O-dimethyl O-(3-methyl-4-nitrophenyl) phosphorothioate, O-ethyl S-phenyl ethylphosphonodithioate, isopropyl 2-((ethoxy(isopropylamino)phosphorothioyl)oxy)benzoate, diethyl 2-((dimethoxyphosphorothioyl)thio)succinate, O,S-dimethyl phosphoramidothioate, O,S-dimethyl phosphoramidothioate, S-((5-methoxy-2-oxo-1,3,4-thiadiazol-3(2H)-yl)methyl) O,O-dimethyl phosphorodithioate, methyl 3-((dimethoxyphosphoryl)oxy)but-2-enoate, (E)-dimethyl (4-(methylamino)-4-oxobut-2-en-2-yl) phosphate, 1,2-dibromo-2,2-dichloroethyl dimethyl phosphate, isopropyl (S)-methylphosphonofluoridate, 3,3-dimethylbutan-2-yl (S)-methylphosphonofluoridate, O,O-diethyl O-(4-nitrophenyl) phosphorothioate, S-(2-(ethylsulfinyl)ethyl) O,O-dimethyl phosphorothioate, O,O-diethyl S-((ethylthio)methyl) phosphorodithioate, S-((6-chloro-2-oxobenzo[d]oxazol-3(2H)-yl)methyl) O,O-diethyl phosphorodithioate, S-((1,3-dioxoisoindolin-2-yl)methyl) O,O-dimethyl phosphorodithioate, (E)-3-chloro-4-(diethylamino)-4-oxobut-2-en-2-yl dimethyl phosphate, O,O,O′,O′-tetramethyl O,O′-(thiobis(4,1-phenylene)) bis(phosphorothioate), tetraethyl diphosphate, S-((tert-butylthio)methyl) O,O-diethyl phosphorodithioate, 2-chloro-1-(2,4,5-trichlorophenyl)vinyl dimethyl phosphate, and dimethyl (2,2,2-trichloro-1-hydroxyethyl)phosphonate, or pharmaceutically acceptable derivatives, metabolites, analogs, or salts thereof, prepared using prodrug strategies described in FIG. 13 wherein the parent drug compounds of Formula I and II are represented by R with general schemes representing various embodiments of prodrugs of compounds of Formula I and II

### Methods of Use

The five most costly brain disorders (€ million) were: dementia: €22,164; psychotic disorders: €16,717; mood disorders: €19,238; addiction: €11,719; anxiety disorders: €11,687. Apart from psychosis, these five disorders ranked amongst those with the lowest direct medical expenditure per subject (<€3000). It was estimated that the total cost of bipolar disorder (BP), also known as manic-depressive illness, made more than a decade ago was as high as $45 billion per year. Most of this cost is accounted for by indirect costs related to the reduced functional capacity and lost work. Patients with BP have higher rates of utilization of healthcare resources compared with the general population and compared with patients with other types of psychiatric conditions. Comorbidity contributes to the heavy burden that BP imposes on society. Brain diseases represent a considerable social and economic burden in Europe. With yearly costs of about 800 billion euros and an estimated 179 million people afflicted in 2010, brain diseases are an unquestionable emergency and a grand challenge for neuroscientists. The global cost of mental health conditions alone was estimated at US$2.5 trillion in 2010, with a projected increase to over US $6 trillion in 2030. Glioblastoma multiform is the most common malignant primary brain tumor in adults, with an estimated incidence of 4.43 per 100,000 person-years in the United States and a median age at presentation of 64 years. Symptoms often include headaches; nausea and vomiting; and progressive memory, personality, or neurologic deficits. While Alzheimer and other dementias are projected to show a 66% increase from 2005 to 2030. In the United States, depression is the second highest source of disability among women, and antidepressant non-responders are among the heaviest users of health care resources. Despite the clear decrease in quality of life and decreased productivity associated with depression, it is often underdiagnosed and inadequately treated.

Drug and alcohol dependence is a severe public health problem. It is estimated that between 26.4 million and 36 million people abuse opioids worldwide (UNODC, World Drug Report 2012), with an estimated 2.1 million people in the United States suffering from substance use disorders related to prescription opioid pain relievers in 2012 and an estimated 467,000 addicted to heroin (Substance Abuse and Mental Health Services Administration, Results from the 2012 National Survey on Drug Use and Health: Summary of National Findings, NSDUH Series H-46, HHS Publication No. (SMA) 13-4795. Rockville, Md.: Substance Abuse and Mental Health Services Administration, 2013; incorporated by reference in its entirety). The number of unintentional overdose deaths from prescription pain relievers has soared in the United States, more than quadrupled since 1999. There is also growing evidence to suggest a relationship between increased non-medical use of opioid analgesics and heroin abuse in the United States.

Links between a chronic diabetic metabolic situation and the risk and emergence of AD pathophysiology have long been suspected and substantiated in the recent years. In several large post-mortem series, more than a third of all subjects clinically diagnosed with typical AD showed evidence of cerebrovascular disease and had to be re-classified as mixed dementia. From a clinical perspective, it is therefore desirable to extend AD therapy beyond currently approved drugs and mechanisms, and also address the cognitive impairment by optimizing a latent diabetic metabolic situation or the fairly frequent Type 2 diabetes in the elderly subjects. Indeed, glycemic control is thought to have an impact on the severity of cognitive impairment. Due to the specific anti-diabetic actions of a compound of Formula I described above, in one embodiment, the present invention provides benefit on both, symptoms and disease progression in AD, and in cognitive impairment of mainly vascular origin (multi-infarct dementia, vascular dementia, vascular cognitive impairment, etc.).

In Parkinson's Disease, the anticholinergic effects of neuroleptics are highly unwanted as they inevitably worsen, in addition the motor condition and symptoms of the vegetative nervous system. In all dementias, lowering the seizure threshold is another infrequent but highly unwanted potential adverse effect of neuroleptics. About 10 million people worldwide have Parkinson's disease. Parkinson's disease is a synucleinopathy resulting in progressive neurodegeneration marked by motor dysfunction and non-motor symptoms including psychosis. More than 50% of patients with Parkinson's disease have psychosis at some time. Psychosis affects up to 75% of patients with Parkinson's disease dementia, and symptoms are more intractable in this group. Such psychosis is expressed primarily as hallucinations and delusions, which can cause great distress for patients and their caregivers. These episodes present a major challenge for treatment and care, increase the likelihood of placement in nursing homes, and are associated with increased mortality. Best-practice treatment guidelines promote initial consideration of comorbidities and reduction of dopaminergic therapy. However, these approaches are often insufficient, and few other therapeutic options exist.

The morbidity and mortality associated with depression are considerable and continue to increase. Depression currently ranks fourth among the major causes of disability worldwide, after lower respiratory infections, perinatal conditions, and HIV/AIDS. Seventeen percent of people will suffer from depression during their lifetime; making matters worse, people already suffering either from acute or chronic illness are even more likely to suffer from depression, where the incidence of depression may be 30% to 50% in patients depending on the specific medical condition.

The monoamine hypothesis has been the prevailing hypothesis of depression over the last several decades. It states that depression is associated with reduced monoamine function. Hence efforts to increase monoamine transmission by inhibiting serotonin (5-HT) and norepinephrine (NE) transporters has been a central theme in depression research since the 1960s. The selective 5-HT reuptake inhibitors (SSRIs) and 5-HT and NE reuptake inhibitors (SNRIs) that have emerged from this line of research are currently first line treatment options for major depressive disorder (MDD). One of the recent trends in antidepressant research has been to refine monoaminergic mechanisms by targeting monoaminergic receptors and additional transporters (e.g. with multimodal drugs and triple re-uptake inhibitors) or by adding atypical antipsychotics to SSRI or SNRI treatment. In addition, several other hypotheses of depression have been brought forward in pre-clinical and clinical research based on biological hallmarks of the disease and efficacy of pharmacological interventions. A central strategy has been to target glutamate receptors (for example, with intravenous infusions of the N-methyl-D-aspartate (NMDA) receptor antagonist ketamine). Other strategies have been based on modulation of cholinergic and gamma-aminobutyric acid (GABA)ergic transmission, neuronal plasticity, stress/hypothalamic pituitary adrenal (HPA)-axis, the reward system and neuroinflammation. Thus, there is a need to develop novel medications with complex pharmacological profiles derived from targeting several neurotransmitter and neuromodulator systems simultaneously.

BP frequently occurs together with other psychiatric disorders, especially anxiety disorders and substance abuse. In addition, BP has been associated with a variety of general medical conditions, which further complicate management of the psychiatric disorder.

BP is a brain disorder that causes unusual shifts in mood, energy, activity levels, and the ability to carry out day-to-day tasks. BP is characterized by a dysregulation of mood, impulsivity, risky behavior and interpersonal problems. BP is a recurrent and often chronic psychiatric illness, associated with functional impairment, elevated suicide rates and utilization of mental health systems. BP is commonly under-recognized and as many as 40% of patients with BPs are initially misdiagnosed, resulting in increased risk for suicide, mania and chronic psychosocial suffering. When correctly diagnosed, successful treatment is possible <50% of diagnosed patients and as many as 10-15% of patients eventually die as a result of suicide.

While the pharmacological guidelines for treatment are well established, treatment for BP remains less than ideal. Most individuals still have breakthrough episodes or significant residual symptoms while on medication. In addition, functional deficits often remain even when patients are in remission. Because many patients with BP remain symptomatic, even while fully adherent to their medication regimens, the need for greater understanding of the pathogenesis of this illness from the research on the pharmacological mechanisms of bipolar medications is all the more urgent. The major medication therapy of BPs is mood stabilizers unless the pharmacology mechanisms are not clear yet. Common neuroprotective effects of mood stabilizers play a role of brain cell dysfunction in BP, and the dysfunction may eventually cause neuron loss. Volumetric neuroimaging, now increasingly assessing potential involvement of different brain structures in mood regulation, could be applied to test neuroanatomical models of mood disorders. Imaging studies suggested that ongoing neuronal atrophy accompanies BP. For instance, PET images of the cerebral blood flow and the rate of glucose metabolism regarding as brain activity detected the reduced activity in the subgenual prefrontal cortex during the bipolar depression. This decrement in activity was, in part, at least explained by a corresponding reduction of cortical volume, as same as magnetic resonance imaging demonstration of the mean gray matter volume. In BP, abnormalities of the third ventricle, frontal lobe, cerebellum, and possibly the temporal lobe are also noted.

Brain tumors are formed by abnormal growths and can appear in different areas of the brain. Benign (not cancerous) tumors may grow and press on nearby areas of the brain, but rarely spread into other tissues. Malignant (cancerous) tumors are likely to grow quickly and spread into other brain tissue. A tumor that grows into or presses on an area of the brain may stop that part of the brain from working the way it should, whether the tumor itself is benign or malignant, and will then require treatment. The most common type of brain tumor seen does not originate from the brain tissue itself, but rather are metastases from extracranial cancers such as lung cancer and breast cancer. Brain tumors include neurofibromatosis type 1 or 2, von Hippel-Lindau disease, tuberous sclerosis, Li-Fraumeni syndrome, Turcot syndrome type 1 and type 2, Klinefelter syndrome, and Nevoid basal cell carcinoma syndrome. Neuroblastoma is cancer found in developing nerve cells, usually in children under 10 years of age. Almost 90% of cases are diagnosed by the age of 5. Different factors can affect the type of neuroblastoma a child has and their prognosis.

Specific treatment for neurological cancer is based on several factors including a patient's overall health and medical history; the type, location, and size of the tumor; the extent of the condition; and other individual factors. Generally, treatment for patients with cancer of the brain or spinal cord includes surgery, chemotherapy, radiation therapy, and/or steroids to treat and prevent swelling, especially in the brain; anti-seizure medication to treat and prevent seizures associated with intracranial pressure; placement of a shunt (to help drain excess fluid in the brain); lumbar puncture/spinal tap (to measure pressure in the spinal cord and brain); bone marrow transplantation; rehabilitation (to regain lost motor skills and muscle strength); and/or antibiotics (to treat and prevent infections). Chemotherapy is the use of anticancer drugs to treat cancerous cells. In most cases, chemotherapy works by interfering with the cancer cell's ability to grow or reproduce. These drugs may be given into a vein or by mouth, as a tablet.

Neuropsychiatric symptoms are a common burden in patients suffering from Alzheimer's disease (AD), Parkinson's disease dementia (PDD), and many other neurodegenerative disorders, including but not limited to dementia with Lewy bodies (DLB), vascular dementia (VaD), and frontotemporal lobar degeneration (FTLD).

Many neuropsychiatric symptoms manifest very early in neurodegenerative disease stages, and are even considered prodromal indicators or indicators for disease progression.

Behavioral and psychological symptoms of dementia (BPSD), also known as neuropsychiatric symptoms, in neurodegenerative diseases and disease states including but not limited to AD have a multifactorial origin. Therefore, a strategy aimed at simultaneously targeting multiple etiologies of a disease (hence, multiple drug targets) constitutes the best approach in the development of treatment strategies for a range of diseases including but not limited to AD.

Individual BPSD symptoms may appear as mutually exclusive but can nevertheless share the underlying mechanisms. This shared mechanism similarity can occur at the neurochemical and/or neuroanatomical levels and serves as a basis for developing targeted, but not mechanism-specific therapies addressing more than one BPSD symptom.

Shared mechanisms are illustrated by similar neurochemical organizations of the projections from cortical areas to basal ganglia to thalamus and back to the cortex. For example, the dorsolateral prefrontal cortex projects to the dorsolateral caudate which in turn targets lateral dorsomedial parts of internal globus pallidus that sends projections to the principal part of the ventral anterior or mediodorsal thalamus which returns projections to the cortex. In contrast, the orbitofrontal cortex projects to the ventromedial caudate that projects to medial dorsomedial parts of internal globus pallidus that sends projections to the magnocellular part of ventral anterior or mediodorsal thalamus which returns projections to the cortex. Thus, different parts of cortex may be responsible for different functions but there are common principles according to which cortical networks operate. For example, the dorsal anterior cingulate cortex and dorsolateral prefrontal cortex are more affected in apathetic patients, and the medial orbital frontal cortex in disinhibited patients with bvFTLD.

Compounds of Formula I, including but not limited to, CBD has been shown to have analgesic, anticonvulsant, muscle relaxant, anxiolytic, neuroprotective, anti-oxidant, and antipsychotic activity. This wide variety of effects is likely due to its complex pharmacological mechanisms. CBD acts on cannabinoid (CB) receptors of the endocannabinoid system, which are found in numerous areas of the body, including the peripheral and central nervous systems, including the brain. The endocannabinoid system regulates many physiological responses of the body including pain, memory, appetite, and mood. More specifically, CB1 receptors can be found within the pain pathways of the brain and spinal cord where they may affect CBD-induced analgesia and anxiolysis, and CB2 receptors have an effect on immune cells, where they may affect CBD-induced anti-inflammatory processes. CBD has been shown to act as a negative allosteric modulator of the cannabinoid CB1 receptor, the most abundant G-Protein Coupled Receptor (GPCR) in the body. Allosteric regulation of a receptor is achieved through the modulation of the activity of a receptor on a functionally distinct site from the agonist or antagonist binding site. The negative allosteric modulatory effects of CBD are therapeutically important as direct agonists are limited by their psychomimetic effects while direct antagonists are limited by their depressant effects. In addition to binding to CB1 and CB2 receptors of the endocannabinoid system, there is evidence that CBD activates 5-HT_{1A} serotonergic and TRPV1-2 vanilloid receptors, antagonizes alpha-1 adrenergic and μ-opioid receptors, inhibits synaptosomal uptake of noradrenaline, dopamine, serotonin and gaminobutyric acid and cellular uptake of anandamide, acts on mitochondria Ca2 stores, blocks low-voltage-activated (T-type) Ca2 channels, stimulates activity of the inhibitory glycine-receptor, and inhibits activity of fatty amide hydrolase (FAAH). CBD are metabolized in the liver by a number of cytochrome P450 isoenzymes, including CYP2C9, CYP2C19, CYP2D6 and CYP3A4. The main primary metabolite of CBD is 7-hydroxy-cannabidiol. CBD is a potent competitive inhibitor for CYP2D6. Other cannabidiols derivatives include 6a-hydroxycannabidiol, 7-hydroxycannabidiol, etc. Two free phenolic hydroxyl groups in theresorcinol moiety and the pentyl side chain of CBD are required for CYP2D6 inhibition.

For glutamatergic signaling that is targeted by dextromethorphan and memantine, it is well established that it mediates thalamocortical signaling, causing the activation of corresponding areas of the cortex.

Diseases like Alzheimer's disease are characterized by systematic, progressive, probably trans-synaptic spread of neurodegeneration. That does not only mean more cell loss in a certain area of the brain but also spreading of the pathology to other brain areas. As different brain areas have different functional roles, this explains why more advanced stages of the disease are accompanied by a wider spectrum of symptoms.

Behavioral and psychological symptoms of dementia, also known as neuropsychiatric symptoms, are commonly studied in the clinic using research tools such as the Neuropsychiatric Inventory (NPI). The NPI recognizes 12 sub-domains of behavioral functioning: delusions, hallucinations, agitation/aggression, dysphoria, anxiety, euphoria, apathy, disinhibition, irritability/lability, aberrant motor activity, night-time behavioral disturbances, and appetite and eating abnormalities.

Patients rarely display each and every of these NPI symptoms at once as there are NPI items like euphoria that are rare, even at a CDR score of 3. Conversely, clinical experience indicates that there is rarely a patient showing just one specific item, and none of the rest. Instead, BPSD symptoms occurs in various combinations or clusters. For example, a frequent AD cluster could e.g. be aggression, agitation, wandering, repetitiveness, while a frequent Vascular Dementia cluster could e.g. be confusion and restlessness, but the frequency and severity of NPI items is subject to change, e.g. from day to day, but especially during disease progression. As a given patient may present such a cluster of several symptoms of clinical relevance at once, there is a high medical need in treatments that can target various clusters of symptoms or the entire range of BPSD symptoms, irrespective of any currently prevailing pathophysiological hypothesis on the disease.

The prevalence of delusions is rather low in general population, in people with normal cognitive aging (0.4-2.4%) but is increased in subjects with mild cognitive impairment (MCI; 3.1-3.4%) and markedly increased in dementia (18.0-31.0%). Prevalence of hallucinations is also low in the general population, in people with normal cognitive aging (0.4-0.6%) but is increased in subjects with MCI (0.6-1.3%) and dementia (10.5-16.0%).

Both delusions and hallucinations are part or symptoms of psychosis in various neurological and psychiatric diseases and disease states. Neuroleptics have traditionally been used off-label to treat such symptoms faute-de-mieux in dementia; however, with very few exceptions both “typical” and “atypical” neuroleptics increase the incidence of CV adverse events and showed a markedly increased death rate when used off-label in dementia. Hence, the FDA issued a “black box” warning against their off-label use outside schizophrenia which leaves little therapeutic options to treat such BPSD symptoms in dementia. On this background, a drug with a completely different mechanism of action, namely cannabidiol, demonstrated an antipsychotic-like efficacy profile in preclinical studies and was suggested to have antipsychotic effects with relatively few adverse effects in clinical studies involving patients with psychosis.

Dextromethorphan has NMDA receptor channel blocking properties and NMDA receptor channel blockers such as phencyclidine or ketamine are known to possess psychotomimetic rather than antipsychotic properties. There are reports of psychosis induced by dextromethorphan in humans. These psychoactive properties of dextromethorphan may be a function of its metabolic degradation resulting in production of dextrorphan. Psychoactive effects of dextromethorphan observed in some subjects do not exclude a possibility that dextromethorphan also has antipsychotic properties under certain circumstances. Indeed, dextromethorphan, but not its metabolite dextrorphan, was reported to attenuate phencyclidine-induced motor behaviors in rats. Meta-analysis of the randomized controlled studies of another NMDA receptor channel blocker, memantine, in patients with Alzheimer's disease indicated that memantine induces significant improvement in delusions.

Agitation and aggression are grouped together as one item on the NPI scale. Prevalence of agitation and aggression is low in general population, in people with normal cognitive aging (2.8-2.9%) but is increased in subjects with MCI (9.1-11.3%) and dementia (30.3-40%). So this NPI item is one the most prevalent and at the same time difficult to treat clinical BPSD symptom.

Various NMDA receptor channel blockers have been shown to attenuate aggressive behaviors in mice and these effects may be difficult to separate from sedative action. In patients with probable Alzheimer disease and clinically significant agitation, dextromethorphan-quinidine combination reduced Agitation/Aggression scores of the NPI. A meta-analysis of randomized controlled studies of another nonselective NMDA receptor channel blocker, memantine, in patients with Alzheimer's disease indicated that also memantine induces significant improvement in agitation/aggression.

The prevalence of dysphoria/depression is moderate in general population, in people with normal cognitive aging (7.2-11.4%) but is increased in subjects with MCI (20.1-27.0%) and it is one of the most prevalent problems in dementia (32.3-42%).

NMDA receptor channel blockers such as dextromethorphan have been shown to possess antidepressant-like properties in preclinical models. Of the NMDA receptor channel blockers, ketamine, is proven to have rapid and robust antidepressant activity in patients with treatment-resistant major depressive disorder. Dextromethorphan given in combination with quinidine also exerts antidepressant action in humans. Dextromethorphan is not a selective NMDA receptor channel blocker and is more potent at serotonin and norephinephrine transporters as well as sigma-1 receptors that may contribute to therapeutic effects of dextromethorphan. While monoamine transporters are targeted by most currently used antidepressants, sigma-1 receptors have also been found to contribute to antidepressant-like effects of dextromethorphan in laboratory animals.

The prevalence of apathy is low in general population, in people with normal cognitive aging (3.2-4.8%) but is increased in subjects with MCI (14.7-18.5%) and dementia (35.9-49%). NMDA receptor channel blockers such as memantine are reported to reduce apathy in certain patients with neurodegenerative diseases.

The prevalence of anxiety is low in general population, in people with normal cognitive aging (5.0-5.8%) but is increased in subjects with MCI (9.9-14.1%) and dementia (21.5-39%).

CBD elicits anxiolytic effects comparable to those of classical anxiolytic drugs (e.g. diazepam) in animal anxiety models, including the marble-burying test, the Vogel conflict test and the elevated plus maze. In humans, the anxiolytic effects of CBD have been supported through tasks involving anxiogenic public speech paradigms in healthy individuals, in social anxiety, in clinical high risk of psychosis and in Parkinson's disease. Further, CBD also reduced drug cue-induced anxiety in heroin-abstinent individuals.

Like other members of the NMDA receptor antagonist class, dextromethorphan was observed to induce anxiolytic-like effects in laboratory animals within a certain dose range. Preclinical anxiolytic effects of dextromethorphan may be related not only to the inhibition of NMDA receptor function but also to interaction with the sigma-1 receptors. In patients with AD, treatment with another nonselective NMDA receptor channel blocker, memantine, significantly decreases in the scores of NPI subscale for anxiety.

The prevalence of euphoria/elation is very low in general population, and in people with normal cognitive aging (0.3-0.4%) but is increased in subjects with MCI (0.6-1.3%) and dementia (3.1-7%).

Human PET studies have established a positive correlation of the psychostimulant drug-induced-induced changes in euphoria analog scale scores with decreases in [11C]raclopride receptor binding potential (BP) in the caudate nucleus and putamen consistent with an increase in endogenous dopamine. Dopaminergic midbrain system is also under control of cholinergic projections such as those originating in habenula and activity of these projections are modulated by α3β4-containing nicotinic acetylcholine receptors. Antagonism at α3β4-containing nicotinic acetylcholine receptors is associated with various effects ascribed to reduced dopamine tone. α3β4-containing nicotinic acetylcholine receptors are one of the main targets of dextromethorphan.

The prevalence of disinhibition is low in general population, in people with normal cognitive aging (0.9-1.6%) but is increased in subjects with MCI (3.1-4.7%) and dementia (12.7-17%). Combination of dextromethorphan and quinidine has positive therapeutic effects in patients with pseudobulbar affect (PBA). PBA may occur in association with a variety of neurological diseases such as amyotrophic lateral sclerosis, extrapyramidal and cerebellar disorders, multiple sclerosis, traumatic brain injury, Alzheimer's disease, stroke, and brain tumors. PBA is a disinhibition syndrome, in which pathways involving serotonin and glutamate are disrupted. Meta-analysis of the randomized controlled studies of another nonselective NMDA receptor channel blocker, memantine, in patients with Alzheimer's disease indicated that memantine induces significant improvement in disinhibition.

The prevalence of irritability/lability is low in general population, in people with normal cognitive aging (4.6-7.6%) but is increased in subjects with MCI (14.7-19.4%) and dementia (27-36%). Combination of dextromethorphan and quinidine has positive therapeutic effects in patients with pseudobulbar affect that is characterized by emotional lability, uncontrolled crying or laughing which may be disproportionate or inappropriate to the social context. In patients with AD, treatment with another nonselective NMDA receptor channel blocker, memantine, significantly decreases the scores of NPI item for irritability/lability. Meta-analysis of the randomized controlled studies of memantine in patients with Alzheimer's disease indicated that memantine was superior to control in irritability/lability.

The prevalence of aberrant motor activity is low in general population, in people with normal cognitive aging (0.4-0.6%) but is increased in subjects with MCI (1.3-3.8%) and dementia (16-32%). Aberrant motor behavior in various neurological disease state such as Parkinson's disease are due to abnormal plasticity processes in basal ganglia that may be expressed as behavioral sensitization that is sensitive to glutamate/NMDA receptor blockade and antagonism at α3β4-containing receptors two of the receptor targets of dextromethorphan.

The prevalence of night-time behavioral disturbances is moderate in general population, in people with normal cognitive aging (10.9%) but is increased in subjects with MCI (13.8-18.3%) and dementia (27.4-39%). Meta-analysis of the randomized controlled studies of another nonselective NMDA receptor channel blocker, memantine, in patients with Alzheimer's disease indicated that memantine induces significant improvement in nighttime disturbance/diurnal rhythm disturbances. In patients with AD, memantine was effective in reducing fragmented sleep and polysomnography revealed longer total sleep, increases in sleep efficiency and time spent in stage II, and decreases in nocturnal awakening, the periodic limb movement index, and time spent in stage I.

The prevalence of appetite and eating abnormalities is low in general population, in people with normal cognitive aging (5.3%) but is increased in subjects with MCI (10.4-10.7%) and dementia (19.6-34%).

An embodiment is a method of treating behavioral and psychological symptoms of dementia in a patient in need thereof comprising the step of administering a pharmaceutical composition comprising a compound of Formula II and one or more agents are compounds of Formula I.

Dextromethorphan acts at 61 receptor and is an N-methyl-D-aspartate (NMDA) antagonist, and an α3β4 nicotinic receptor antagonist. Uptake of norepinephrine and serotonin are also inhibited. Several neuropsychiatric diseases and syndromes such as Alzheimer's disease and behavioral and psychological symptoms of dementia involve dis-regulation of glutamatergic, cholinergic, serotoninergic and norepinephrinergic neurotransmitter systems. Accordingly, in another embodiment, the composition comprises an NMDA receptor antagonist such as ketamine, methadone, memantine, amantadine, dextropropoxyphene, ketobemidone and dextromethorphan. In another embodiment, the composition is a combination of a compound of Formula I, and ketamine, methadone, memantine, amantadine, dextropropoxyphene, ketobemidone, or dextromethorphan. The compound of Formula I forms the combination as mixture, complex, conjugate, compound with covalent bond, or a salt.

Psychological symptoms of dementia involve disregulation of glutamatergic, cholinergic, serotoninergic and norepinephrinergic neurotransmitter systems. Therefore, an embodiment is a method of treating behavioral and psychological symptoms of dementia. Another embodiment is a treatment of a person in need thereof comprising administering a composition comprising a compound of Formula I and a compound of Formula II to improve EEG abnormalities, behavior, cognition, and reduce seizures, as well as improve breathing abnormalities, motor capabilities, bone density, and GI dysfunction.

Dextromethorphan is metabolized into active metabolites in the liver starting with O- and N-demethylation to form primary metabolites DO and 3-methoxy-morphinan are further N- and O-demethylated respectively to 3-hydroxy-morphinan. A major metabolic catalyst is the cytochrome P450 enzyme 2D6 (CYP2D6), which is responsible for the O-demethylation reactions of dextromethorphan and 3-methoxymorphinan. N-demethylation of dextromethorphan and DO are catalyzed by enzymes in the related CYP3A family. Conjugates of DO and 3-hydroxymorphinan can be detected in human plasma and urine within hours of its ingestion. DO is a substance most notable for its psychoactive effects.

Another embodiment is the method of treatment wherein the patient is suffering from a disease or disorder comprising peripheral arterial disease, e.g., Raynaud's Disease and claudicatio intermittens; pulmonary hypertension, angina pectoris, and/or diabetes mellitus. In another embodiment, the method of treatment of a patient after coronary stenting comprising a compound of Formula I, to and is useful in restenosis.

DO is a substance most notable for its psychoactive effects that likely arise from blockade of NMDA receptors. DO has a substantially higher affinity for NMDA receptors compared to that of dextromethorphan. Adverse psychoactive effects of dextromethorphan have been associated with its metabolism to DO. Therefore, another embodiment is a method of reducing adverse effects of dextromethorphan during treatment of a patient in need thereof comprising the step of administering a pharmaceutical composition comprising dextromethorphan and one or more agents of compounds of Formula I.

The genetically polymorphic cytochrome CYP2D6 has been implicated in the metabolism of many antipsychotic agents, including thioridazine, perphenazine, chlorpromazine, fluphenazine, haloperidol, zuclopenthixol, risperidone, and sertindole (Michalets, 1998). This enzyme is also important in the metabolism of other drugs that are commonly prescribed to patients with psychiatric disorders, e.g., tricyclic antidepressants (nortriptyline, desipramine, amitriptyline, imipramine, and clomipramine) and selective serotonin reuptake inhibitors, including fluoxetine and paroxetine. Drugs that inhibit these enzymes would be expected to cause increases in the plasma concentration of co-administered antipsychotic drugs. These increases may, in turn, lead to the development or aggravation of antipsychotics-induced side effects including cardiac toxicity, anticholinergic side effects, or orthostatic hypotension.

Many antipsychotic drugs inhibited CYP2D6-catalyzed dextromethorphan O-demethylation. Among the antipsychotic drugs tested, thioridazine and perphenazine were the most potent inhibitors and decreased the DO formation rate to 26.5 and 19.7% of control activity at 10 microM, and 11.4 and 10.7% of control activity at 25 micro M, respectively. The inhibitory potency of these drugs on dextromethorphan O-demethylation was comparable to the inhibitory effect of 10 to 25 microM quinidine. The estimated mean IC50 values for thioridazine and perphenazine were 2.7±0.5 and 1.5±0.3 micro M, respectively. The IC50 of quinidine, a potent CYP2D6 inhibitor, was estimated to be 0.52±0.2 micro M under these conditions. The estimated IC50s of chlorpromazine, fluphenazine, and haloperidol were 9.7, 16.3, and 14.4 micro M, respectively. Cisthiothixene, clozapine, and risperidone exhibited weaker inhibition than the other drugs tested, with mean IC₅₀s estimated to be 136.6, 92.2, and 39.1 micro M, respectively.

In one embodiment, the pharmaceutical composition of the invention comprise one or more of the CYP2D6 inhibitors such as, but are not limited to, Ajmaline, Amiodarone, Amitriptyline, Aprindine, Azelastine, Celecoxib, Chlorpheniramine, Chlorpromazine, Diphenhydramine, Doxorubicin, Fluoxetine, Fluphenazine, Fluvastatin, Fluvoxamine, Haloperidol, Imipramine, Indinavir, Lasoprazole, Levomepromazine, Lopinavir, Loratadine, Mequitazine, Methadone, Metoclopramide, Mibefradil, Moclobemide, Nelfinavir, Nevirapine, Nicardipine, Norfluoxetine, Paroxetine, Perphenazine, Pimozide, Terfenadine, Thioridazine, Cimetidine, Quinidine, Cisapride, Citalopram, Clomipramine, Clozapine, Cocaine, Desipramine, Ranitidine, Risperidone, Ritonavir, Saquinavir, Sertraline, Terbinafine, Ticlopidine, Trifluperidol, Venlafaxine, and Yohimbine.

Some embodiments include a method of decreasing the number of doses and/or total daily dose of dextromethorphan, a metabolite, a derivative or a prodrug thereof that can be administered while increasing efficacy and safeguarding tolerability and safety, comprising orally administering an effective amount of a compound of Formula I and a compound of Formula II.

Another embodiment is a method of treating a neurological disorder comprising administering about 5 mg/day to about 600 mg/day, about 5 mg/day to about 300 mg/day, about 5 mg/day to about 400 mg/day, about 5 mg/day to about 500 mg/day, about 5 mg/day to about 600 mg/day, about 5 mg/day to about 1,000 mg/day, about 50 mg/day to about 1000 mg/day, about 100 mg/day to about 1000 mg/day, about 150 mg/day to about 1000 mg/day, about 150 mg/day to about 5000 mg/day, about 150 mg/day to about 300 mg/day, or about 150 mg/day to about 100 mg/day, or an amount as required of a compound of Formula I and about 0.1 mg/day to about 1 mg/day, about 0.5 mg/day to about 15 mg/day, about 15 mg/day to about 60 mg/day, about 15 mg/day to about 120 mg/day, about 0.1 mg/day to about 200 mg/day, or an amount as required of the composition of the invention to a subject in need thereof.

Another embodiment is a method of increasing dextromethorphan plasma levels in a subject in need of treatment with dextromethorphan, wherein the subject is an extensive metabolizer of dextromethorphan, comprising co-administering a compound of Formula I with dextromethorphan to the subject.

Another embodiment is a method of inhibiting the metabolism of dextromethorphan, comprising administering a compound of Formula I, to a subject, wherein the subject is an extensive metabolizer of dextromethorphan, and wherein dextromethorphan is present in the body of the subject at the same time as a compound of Formula I.

Another embodiment is a method of increasing the metabolic lifetime of dextromethorphan, comprising administering a compound of formula I, to a subject in need of treatment with dextromethorphan wherein the subject is an extensive metabolizer of dextromethorphan, and wherein dextromethorphan is present in the body of the subject at the same time as a compound of formula I.

Another embodiment is a method of increasing dextromethorphan plasma levels comprising co-administering a compound of formula I and dextromethorphan to a subject in need of treatment with dextromethorphan, wherein the compound of formula I is administered on the first day of at least two days of co-administration of a compound of formula I, with dextromethorphan, wherein an increase in the dextromethorphan plasma level occurs on the first day that a compound of formula I and dextromethorphan are co-administered, as compared to the same amount of dextromethorphan administered without a compound of formula I.

Another embodiment is a method of increasing dextromethorphan plasma levels comprising co-administering a compound of formula I and dextromethorphan for at least five consecutive days, to a subject in need of treatment with dextromethorphan, wherein, on the fifth day, the dextromethorphan plasma level is higher than the dextromethorphan plasma level that would have been achieved by administering the same amount of dextromethorphan administered without a compound of formula I, for five consecutive days.

Another embodiment is a method of increasing dextromethorphan plasma levels comprising co-administering a compound of formula I and dextromethorphan for at least six consecutive days, to a subject in need of treatment with dextromethorphan, wherein, on the sixth day, the dextromethorphan plasma level is higher than the dextromethorphan plasma level that would have been achieved by administering the same amount of dextromethorphan administered without a compound of formula I, for six consecutive days.

Another embodiment is a method of reducing a trough effect of dextromethorphan comprising, co-administering a compound of formula I, with dextromethorphan to a subject in need of treatment with dextromethorphan, wherein dextromethorphan has a plasma level 12 hours after co-administering a compound of formula I, with dextromethorphan that is at least twice the plasma level that would be achieved by administering the same amount of dextromethorphan without a compound of formula I.

Another embodiment is a method of reducing a trough effect of dextromethorphan comprising, co-administering a compound of formula I, with dextromethorphan to a subject in need of treatment with dextromethorphan, wherein dextromethorphan has a plasma level 12 hours after co-administering a compound of formula I, with dextromethorphan that is at least twice the plasma level that would be achieved by administering the same amount of dextromethorphan without a compound of Formula I.

Another embodiment is a method of reducing a trough effect of dextromethorphan comprising, co-administering a compound of formula I, with dextromethorphan to a subject in need of treatment with dextromethorphan, wherein dextromethorphan has a plasma level 12 hours after co-administering a compound of formula I, with dextromethorphan that is at least twice the plasma level that would be achieved by administering the same amount of dextromethorphan without a compound of Formula I.

Another embodiment is a method of reducing an adverse event or other unwanted consequences such as addiction associated with treatment by dextromethorphan, comprising co-administering a compound of Formula I, and dextromethorphan to a subject in need of dextromethorphan treatment, wherein the subject is at risk of experiencing the adverse event as a result of being treated with dextromethorphan.

Another embodiment is a method of reducing an adverse event associated with treatment by a compound of formula I, comprising co-administering dextromethorphan and a compound of formula I, to a subject in need of a compound of Formula I, treatment, wherein the subject is at risk of experiencing the adverse event as a result of being treated with a compound of formula I.

Another embodiment is a method of improving antitussive properties of dextromethorphan comprising administering a compound of formula I, in conjunction with administration of dextromethorphan to a subject in need of treatment for cough. Another embodiment is a method of treating cough comprising administering a combination of a compound of formula I and dextromethorphan to a subject in need thereof. Another embodiment is a method of treating a neurological disorder comprising administering a compound of formula I and dextromethorphan to a subject in need thereof, wherein the compound of formula I and dextromethorphan are administered at least once a day for at least 8 days.

Another embodiment is a method of treating a neurological disorder comprising administering a composition comprising dextromethorphan, Formula I to a subject in need thereof, wherein the composition comprising dextromethorphan and Formula I is administered at least once a day for at least 8 days. Another embodiment is a method of treating a neurological disorder comprising administering a composition comprising dextromethorphan and Formula I to a subject in need thereof, wherein the compound of formula I and dextromethorphan are administered at least once a day for at least 8 days. Another embodiment is a pharmaceutical composition, dosage form, or medicament comprising a therapeutically effective amount of dextromethorphan, a therapeutically effective amount of a compound of formula I and a pharmaceutically acceptable excipient.

In another aspect, provided is a method of preventing adverse events associated with treatment by dextromethorphan, comprising co-administering a compound of formula I, to a subject in need of treatment with dextromethorphan, wherein the subject is at risk of experiencing the adverse event as a result of being treated with dextromethorphan.

Another embodiment, NMDA receptor antagonists reduce the physical aspects of the expression of morphine dependence as measured by naloxone-precipitated withdrawal and may attenuate not only the physical but also affective and motivational components of abstinence states, as well as craving.

Accordingly, an embodiment is a method of treating a subject in need of treatment for disorders or diseases associated with addiction and substance abuse comprising administration of dextromethorphan and Formula I.

Chronic exposure to morphine results in a number of biochemical adaptations of the glutamatergic receptor system in the limbic system. Excitatory amino acids are involved in the mediation of many neurochemical and behavioral effects resulting from chronic exposure to abusing drugs, some of which can be prevented or reversed using glutamatergic antagonists.

Continued self-administration of abusive drugs, including opioid, results in an overstimulation of dopamine in the brain reward centers and an increased release of excitatory amino acids including glutamate leading to the development of tolerance and dependence which could be blocked by glutamate antagonists. Accordingly, an embodiment is a method of treating a subject in need of treatment for disorders or diseases associated with addiction and substance abuse resulting from opioid tolerance and dependence by amelioration of opiate withdrawal symptoms and relapse prevention comprising administration of dextromethorphan and Formula I.

DEX affords neuroprotection on dopamine neurons in several inflammation-based animal Parkinson's disease models. 1-10 microM dextromethorphan protected dopamine neurons against lipopolysaccharide (LPS)-induced reduction of dopamine uptake in rat primary mixed mesencephalic neuron-glia cultures. Morphologically, in LPS-treated cultures, besides the reduction of an abundance of dopamine neurons, the dendrites of the remaining dopamine neurons were significantly less elaborative than those in the controls. In cultures pretreated with dextromethorphan (10 micro M) before LPS stimulation, dopamine neurons were significantly more numerous and the dendrites less affected. Significant neuroprotection was observed in cultures with dextromethorphan added up to 60 minutes after the addition of LPS. Thus, dextromethorphan significantly protects monoamine neurons not only with pretreatment but also with post-treatment. Animal studies using both LPS and MPTP PD models also show potent protective effect of dextromethorphan. Accordingly, an embodiment is a method of treating a subject in need of a treatment for Parkinson's disease comprising administration of dextromethorphan and Formula I.

The neuroprotective effect of dextromethorphan is associated with the inhibition of microglia over-activation by inhibition of superoxide anion production from NADPH-oxidase, and this neuroprotective effect of dextromethorphan is not associated with its NMDA receptor antagonist property. There is A correlation was observed between the anti-inflammatory potency and neuroprotection of NMDA receptor antagonists, such as MK801, AP5, and memantine, suggesting that the dopamine neuroprotection provided by dextromethorphan in the inflammation-related neurodegenerative models is not mediated through the NMDA receptor. This conclusion is not in conflict with previous reports, indicating that NMDA receptor blockade is associated with the neuroprotective effects of dextromethorphan in the acute glutamate-induced excitotoxicity models. Accordingly, an embodiment is a method of treating a subject in need of treatment for a disorder or disease thereof comprising administration of dextromethorphan and Formula I, wherein the disorder or disease is an inflammation-related neurodegenerative disorder.

GC-dependent effects of morphine activate the hypothalamic-pituitary-adrenal (HPA) axis. The activation of the HPA axis increases the products of GC as potent immunomodulatory hormones. Accordingly, an embodiment is a method of treating a subject in need of treatment for a disorder or disease thereof comprising administration of dextromethorphan and Formula I, wherein the disorder or disease is opioid dependence. The dosage of up to about 500 mg/day dextromethorphan is suggested, including doses of 120, 240, and 480 mg/day of dextromethorphan for heroin addicts undergoing withdrawal. dextromethorphan at high doses caused mild elevations of heart rate, blood pressure, temperature, and plasma bromide. Particularly among Han Chinese in Taiwan, dextromethorphan has been reported to have quite different “dextromethorphan metabolic enzyme CYP2D6” from that of Western population.

Significantly higher interleukin-6, interleukin-8, and TNF-alpha levels are manifested in bipolar disorder (BP) patients during manic and depressive episodes than normal controls.

In postmortem frontal cortex from BP patients, the significantly higher protein and mRNA levels of IL-1 beta receptor and neuroinflammatory markers inducible nitric oxide synthase (iNOS) and c-fos were found. Taken together, the imbalance of the immune system, subsequently leading to the neuronal inflammatory response, might be related to the progression of the brain atrophy and aggravated BP symptoms. BP treatment with immune-targeted therapies showed antidepressant effects. For example, open-label acetylsalicylic acid when added to fluoxetine led to increased remission rates in individuals with major depression who were previously non-responsive to fluoxetine monotherapy.

Thus, using an anti-inflammatory agent combined with a mood stabilizer improves the treatment effect on BP. Mood stabilizers have been shown to activate interconnected intracellular signaling pathways that promote neurogenesis and synaptic plasticity. A reduction in brain volume in BP patients was found to be largely suppressed by chronic treatment with Valproate (VPA) resulting in neuroprotective effects, as VPA renders neurons less susceptible to a variety of insults and even stimulates neurogenesis in the adult rodent brain. VPA induces cytoprotective proteins like Bcl-2, glucose-regulated protein 78 (Grp78), brain-derived neurotrophic factor (BDNF) and heat shock protein 70. Moreover, VPA promotes neurite outgrowth, while VPA at therapeutic levels was reported to inhibit histone deacetylase (HDAC), an enzyme that catalyzes the removal of the acetyl group from lysine residues of histones, promoting local, neuronal BDNF biosynthesis. Accordingly, an embodiment is a method of treating a subject in need of treatment for a disorder or disease thereof comprising administration of dextromethorphan and a compound of Formula I, wherein the disorder or disease is BP.

Another embodiment is a method of reducing adverse events of dextromethorphan in a subject in need thereof comprising:

a. administering dextromethorphan; and

b. administering a compound of Formula I, to the subject.

Some embodiments include a method of treating neuropsychiatric disorders comprising administering a therapeutically effective amount of dextromethorphan and a therapeutically effective amount of a compound of formula I, to a person in need thereof.

Some embodiments include a method of enhancing the therapeutic properties of dextromethorphan in treating neuropsychiatric disorders, comprising co-administering dextromethorphan and a compound of formula I.

Dextromethorphan is used as a cough suppressant. According to the FDA's dextromethorphan product labeling requirement under the OTC Monograph [21CFR341.74], dextromethorphan should be dosed 6 times a day (every 4 hours), 4 times a day (every 6 hours), or 3 times a day (every 8 hours).

Dextromethorphan is rapidly metabolized in the human liver. This rapid hepatic metabolism may limit systemic drug exposure in individuals who are extensive metabolizers. Subjects can be: 1) extensive metabolizers of Dextromethorphan those who rapidly metabolize Dextromethorphan; 2) poor metabolizers of Dextromethorphan those who only poorly metabolize Dextromethorphan, or 3) intermediate metabolizers of Dextromethorphan those whose metabolism of dextromethorphan is somewhere between that of an extensive metabolizer and a poor metabolizer. Extensive metabolizers can also be ultra-rapid metabolizers. Extensive metabolizers of dextromethorphan are a significant portion of the human population. Dextromethorphan can, for example, be metabolized to DO.

When given the same oral dose of dextromethorphan, plasma levels of dextromethorphan are significantly higher in poor metabolizers or intermediate metabolizers as compared to extensive metabolizers of dextromethorphan. The low plasma concentrations of dextromethorphan can limit its clinical utility as a single agent for extensive metabolizers, and possibly intermediate metabolizers, of dextromethorphan. Some antidepressants, such as a compound of Formula I inhibit the metabolism of dextromethorphan, and can thus improve its therapeutic efficacy. Similarly, antidepressants may allow dextromethorphan to be given less often, such as once a day instead of twice a day, once a day instead of three times a day, once a day instead of four times a day, twice a day instead of three times a day, or twice a day instead of four times a day, without loss of therapeutic efficacy.

Apathy, or loss of motivation, is the most common change in behavior in Alzheimer's disease (AD). It is common throughout the spectrum of cognitive decline from mild cognitive impairment to severe Alzheimer's disease (AD), as well as in a variety of other neuropsychiatric disorders. Apathy represents a form of executive cognitive dysfunction. Patients with apathy suffer from decreased daily function and specific cognitive deficits and rely on families to provide more care, which results in increased stress for families. Apathy is one of the primary syndromes associated with frontal and subcortical pathology, and apathy in AD appears to have multiple neuroanatomical correlates that implicate components of frontal subcortical networks. Despite the profound effects of this common syndrome, only a few instruments have been designed to specifically assess apathy, and these instruments have not been directly compared. Assessment of apathy in AD requires clinicians to distinguish loss of motivation from loss of ability due to cognitive decline. Although apathy may be misdiagnosed as depression because of an overlap in symptoms, current research has shown apathy to be a discrete syndrome. Distinguishing apathy from depression has important treatment implications because these disorders respond to different interventions.

The Apathy Inventory (IA), a rating scale for global assessment of apathy and separate assessment of emotional blunting, lack of initiative, and lack of interest, is a reliable method for assessing in demented and non-demented elderly subjects several dimensions of the apathetic syndrome, and also the subject's awareness of these symptoms. The IA assesses apathy as effectively as the Neuro Psychiatric Inventory apathy domain. Apathy can be the result of damage to one or more areas of the brain such as the frontal cortex, the thalamus, striatum and the amygdala. In most cases direct damage to the frontal lobes or the subcortical nuclei that have connections to the frontal lobes, cause apathy. Apathy associated with Alzheimer's disease is very difficult to treat. Antidepressants, SSRIs, psychostimulants, acetylcholinesterase inhibitors etc. alleviated apathy only to some degree.

Accordingly, an embodiment of the invention is a combination of dextromethorphan and Formula I, and one or more of antidepressants, SSRIs, psychostimulants, acetylcholinesterase inhibitors, dopaminergic agents. Another embodiment is a combination of dextromethorphan and Formula I, and one or more of donepezil, memantine, amantanidine, bupropion, ropinirole, methylphenidate, amphetamine, modafinil, metrifonate, tacrine, galantamine, rivastigmine, nefiracetam, Ginkgo biloba extract, etc.

Acetylcholinesterase is one of the most prominent constituents of central cholinergic pathways. It terminates the synaptic action of acetylcholine through hydrolysis and yields the choline moiety that is necessary for transmitter recycling. The pathogenesis of Alzheimer's disease (AD) has been linked to a deficiency in the brain neurotransmitter acetylcholine. The efficacy of acetylcholinesterase inhibitors (AChEIs) is attained through their augmentation of acetylcholine-medicated neuron to neuron transmission. This is accomplished by increasing the concentration of acetylcholine through reversible inhibition of its hydrolysis by acetylcholinesterase.

Accordingly, in one embodiment, the composition comprises AChIs such as 2-((1-Benzylpiperidin-4-yl)methyl)-5,6-dimethoxy-2,3-dihydro-1H-inden-1-one (Donepezil), (S)-3-(1-(dimethylamino)ethyl)phenyl ethyl(methyl) carbamate (Rivastigmine), dimethyl (2,2,2-trichloro-1-hydroxyethyl)phosphonate (Metrifonate), dimethyl (2,2,2-trichloro-1-hydroxyethyl)phosphonate (Metrifonate), (4aS,6R,8aS)-3-methoxy-11-methyl-4a,5,9,10,11,12-hexahydro-6H-benzo[2,3]benzofuro[4,3-cd]azepin-6-ol (Galantamine), and 1,2,3,4-tetrahydroacridin-9-amine (Tacrine), O,S-dimethyl acetylphosphoramidothioate, O,O-dimethyl S-((4-oxobenzo[d][1,2,3]triazin-3(4H)-yl)methyl) phosphorodithioate, 2,2-dimethyl-2,3-dihydrobenzofuran-7-yl methylcarbamate, S-(((4-chlorophenyl)thio)methyl) O,O-diethyl phosphorodithioate, 2-chloro-1-(2,4-dichlorophenyl)vinyl diethyl phosphate, O,O-diethyl O-(3,5,6-trichloropyridin-2-yl) phosphorothioate, O-(3-chloro-4-methyl-2-oxo-2H-chromen-7-yl) O,O-diethyl phosphorothioate, 1-phenylethyl (E)-3-((dimethoxyphosphoryl)oxy)but-2-enoate,4-(tert-butyl)-2-chlorophenyl methyl methylphosphoramidate, O,O-diethyl O-(2-(ethylthio)ethyl) phosphorothioate, O,O-diethyl S-(2-(ethylthio)ethyl) phosphorothioate, O,O-diethyl O-(2-isopropyl-6-methylpyrimidin-4-yl) phosphorothioate, 2,2-dichlorovinyl dimethyl phosphate, (E)-4-(dimethylamino)-4-oxobut-2-en-2-yl dimethyl phosphate, O,O-dimethyl S-(2-(methylamino)-2-oxoethyl) phosphorodithioate, S,S′-(1,4-dioxane-2,3-diyl) O,O,O′,O′-tetraethyl bis(phosphorodithioate), O,O-diethyl S-(2-(ethylthio)ethyl) phosphorodithioate, O-ethyl O-(4-nitrophenyl) phenylphosphonothioate, O,O,O′,O′-tetraethyl S,S′-methylene bis(phosphorodithioate), O-ethyl S,S-dipropyl phosphorodithioate, O-(4-(N,N-dimethylsulfamoyl)phenyl) O,O-dimethyl phosphorothioate, O-(4-(N,N-dimethylsulfamoyl)phenyl) O,O-dimethyl phosphorothioate, ethyl (3-methyl-4-(methylthio)phenyl) isopropylphosphoramidate, O,O-dimethyl O-(3-methyl-4-nitrophenyl) phosphorothioate, O-ethyl S-phenyl ethylphosphonodithioate, isopropyl 2-((ethoxy(isopropylamino)phosphorothioyl)oxy)benzoate, diethyl 2-((dimethoxyphosphorothioyl)thio)succinate, O,S-dimethyl phosphoramidothioate, O,S-dimethyl phosphoramidothioate, S-((5-methoxy-2-oxo-1,3,4-thiadiazol-3(2H)-yl)methyl) O,O-dimethyl phosphorodithioate, methyl 3-((dimethoxyphosphoryl)oxy)but-2-enoate, (E)-dimethyl (4-(methylamino)-4-oxobut-2-en-2-yl) phosphate, 1,2-dibromo-2,2-dichloroethyl dimethyl phosphate, isopropyl (S)-methylphosphonofluoridate, 3,3-dimethylbutan-2-yl (S)-methylphosphonofluoridate, O,O-diethyl O-(4-nitrophenyl) phosphorothioate, S-(2-(ethylsulfinyl)ethyl) O,O-dimethyl phosphorothioate, O,O-diethyl S-((ethylthio)methyl) phosphorodithioate, S-((6-chloro-2-oxobenzo[d]oxazol-3(2H)-yl)methyl) O,O-diethyl phosphorodithioate, S-((1,3-dioxoisoindolin-2-yl)methyl) O,O-dimethyl phosphorodithioate, (E)-3-chloro-4-(diethylamino)-4-oxobut-2-en-2-yl dimethyl phosphate, O,O,O′,O′-tetramethyl O,O′-(thiobis(4,1-phenylene)) bis(phosphorothioate), tetraethyl diphosphate, S-((tert-butylthio)methyl) O,O-diethyl phosphorodithioate, 2-chloro-1-(2,4,5-trichlorophenyl)vinyl dimethyl phosphate, and dimethyl (2,2,2-trichloro-1-hydroxyethyl)phosphonate, or pharmaceutically acceptable derivatives, metabolites, analogs, or salts thereof.

As explained above, this inhibition may augment dextromethorphan plasma levels, resulting in additive or synergistic efficacy such as relief of neurological disorders including pain, depression, smoking cessation, etc. Thus, while inhibition of dextromethorphan metabolism is only one of many potential benefits of the combination, co-administration of dextromethorphan with a compound of formula I may thereby enhance the efficacy of a compound of formula I, for many conditions. Co-administration of dextromethorphan with a compound of formula I may enhance the analgesic properties of a compound of formula I for many conditions. Co-administration of dextromethorphan with a compound of formula I may also enhance the antidepressant properties of a compound of formula I for many conditions, including faster onset of action.

Another potential benefit of co-administration of dextromethorphan and a compound of formula I is that it may be useful to reduce the potential for an adverse event, such as drowsiness or confusion, associated with treatment by dextromethorphan. This may be useful, for example, in subjects at risk of experiencing an adverse event as a result being treated with dextromethorphan.

Another potential benefit of co-administration of dextromethorphan and a compound of formula I is that it may be useful to reduce the potential for an adverse event, such as seizure, associated with treatment by a compound of Formula I. This may be useful, for example, in subjects at risk of experiencing the adverse event as a result being treated with a compound of formula I.

With respect to dextromethorphan, a compound of formula I, co-administration may reduce a central nervous system adverse event, a gastrointestinal event, or another type of adverse event associated with any of these compounds. Central nervous system (CNS) adverse events include, but are not limited to, nervousness, dizziness, sleeplessness, light-headedness, tremor, hallucinations, convulsions, CNS depression, fear, anxiety, headache, increased irritability or excitement, tinnitus, drowsiness, dizziness, sedation, somnolence, confusion, disorientation, lassitude, incoordination, fatigue, euphoria, nervousness, insomnia, sleeping disturbances, convulsive seizures, excitation, catatonic-like states, hysteria, hallucinations, delusions, paranoia, headaches and/or migraine, and extrapyramidal symptoms such as oculogyric crisis, torticollis, hyperexcitability, increased muscle tone, ataxia, and tongue protrusion.

Gastrointestinal adverse events include, but are not limited to, nausea, vomiting, abdominal pain, dysphagia, dyspepsia, diarrhea, abdominal distension, flatulence, peptic ulcers with bleeding, loose stools, constipation, stomach pain, heartburn, gas, loss of appetite, feeling of fullness in stomach, indigestion, bloating, hyperacidity, dry mouth, gastrointestinal disturbances, and gastric pain.

In some embodiments, co-administration of a combination of a compound of formula I and dextromethorphan results in pain relieving properties. For example, the combination may have improved pain-relieving properties as compared to a compound of formula I alone or compared to dextromethorphan alone, including potentially faster onset of action.

In some embodiments, the combination may have improved pain relieving properties of at least about 0.5%, at least about 1%, at least about 10%, at least about 20%, at least about 30%, at least about 50%, at least 100%, up to about 500% or up to 1000%, about 0.5% to about 1000%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, or any amount of pain relief in a range bounded by, or between, any of these values, as compared to a compound of formula I alone.

In some embodiments, the combination may have improved pain relieving properties of at least about 0.5%, at least about 1%, at least about 10%, at least about 20%, at least about 30%, at least about 50%, at least 100%, up to about 500% or up to 1000%, about 0.5% to about 1000%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, or any amount of pain relief in a range bounded by, or between, any of these values, as compared to as compared to dextromethorphan alone.

Unless otherwise indicated, any reference to a compound herein, such as dextromethorphan, a compound of Formula I by structure, name, or any other means, includes pharmaceutically acceptable salts; alternate solid forms, such as polymorphs, solvates, hydrates, etc.; tautomers; deuterium-modified compounds, such as deuterium-modified dextromethorphan and a compound of formula I; or any chemical species that may rapidly convert to a compound described herein under conditions in which the compounds are used as described herein. Examples of deuterium modified dextromethorphan and a compound of formula I, include, but are not limited to, those shown below.

A dosage form or a composition may be a blend or mixture of dextromethorphan and a compound that inhibits the metabolism of dextromethorphan, such as a compound of formula I, either alone or within a vehicle. For example, dextromethorphan and a compound of formula I, may be dispersed within each other or dispersed together within a vehicle. A dispersion may include a mixture of solid materials wherein small individual particles are substantially one compound, but the small particles are dispersed within one another, such as might occur if two powders of two different drugs are blended with a solid vehicle material, and the blending is done in the solid form. In some embodiments, dextromethorphan and a compound of formula I, may be substantially uniformly dispersed within a composition or dosage form. Alternatively, dextromethorphan and a compound of formula I, may be in separate domains or phases in a composition or dosage form. For example, one drug may be in a coating, and another drug may be in a core within the coating. For example, one drug may be formulated for sustained release and another drug may be formulated for immediate release.

Some embodiments include administration of a tablet that contains a compound of formula I in a form that provides sustained release and dextromethorphan in a form that provides immediate release or vice versa. While there are many ways that sustained release of a compound of formula I, may be achieved, in some embodiments, a compound of formula I is combined with hydroxypropyl methylcellulose. For example, particles of a compound of formula I hydrochloride could be blended with microcrystalline cellulose and hydroxypropyl methylcellulose to form an admixture of blended powders. This could then be combined with immediate release dextromethorphan in a single tablet.

Therapeutic compounds may be administered by any means that may result in the contact of the active agent(s) with the desired site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

Therapeutic compounds may be administered to a subject in a variety of forms adapted to the chosen route of administration, e.g., orally or parenterally. Parenteral administration in this respect includes administration by the following routes: intravenous, intramuscular, subcutaneous, intraocular, intrasynovial, transepithelial, including transdermal, ophthalmic, sublingual and buccal; topically including ophthalmic, dermal, ocular, rectal and nasal inhalation via insufflation, aerosol and rectal systemic.

The ratio of dextromethorphan to a compound of formula I may vary. In some embodiments, the weight ratio of dextromethorphan to a compound of formula I, may be about 0.1 to about 10, about 0.1 to about 2, about 0.2 to about 1, about 0.1 to about 0.5, about 0.1 to about 0.3, about 0.2 to about 0.4, about 0.3 to about 0.5, about 0.5 to about 0.7, about 0.8 to about 1, about 0.2, about 0.3, about 0.4, about 0.45, about 0.6, about 0.9, or any ratio in a range bounded by, or between, any of these values. A ratio of 0.1 indicates that the weight of dextromethorphan is 1/10 that of a compound of formula I. A ratio of 10 indicates that the weight of dextromethorphan is 10 times that of a compound of formula I.

The amount of dextromethorphan in a therapeutic composition may vary. For example, some liquid compositions may comprise about 0.0001% (w/v) to about 50% (w/v), about 0.01% (w/v) to about 20% (w/v), about 0.01% to about 10% (w/v), about 0.001% (w/v) to about 10% (w/v), about 0.10% (w/v) to about 0.50% (w/v), about 10% (w/v) to about 30% (w/v), about 3% (w/v) to about 5% (w/v), about 5% (w/v) to about 7% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of dextromethorphan.

Some liquid dosage forms may contain about 10 mg to about 500 mg, about 30 mg to about 350 mg, about 50 mg to about 200 mg, about 50 mg to about 70 mg, about 20 mg to about 50 mg, about 30 mg to about 60 mg, about 40 mg to about 50 mg, about 40 mg to about 42 mg, about 42 mg to about 44 mg, about 44 mg to about 46 mg, about 46 mg to about 48 mg, about 48 mg to about 50 mg, about 80 mg to about 100 mg, about 110 mg to about 130 mg, about 170 mg to about 190 mg, about 45 mg, about 60 mg, about 90 mg, about 120 mg, or about 180 mg of dextromethorphan, or any amount of dextromethorphan in a range bounded by, or between, any of these values.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of dextromethorphan.

Some solid dosage forms may contain about 10 mg to about 500 mg, about 30 mg to about 350 mg, about 20 mg to about 50 mg, about 30 mg to about 60 mg, about 40 mg to about 50 mg, about 40 mg to about 42 mg, about 42 mg to about 44 mg, about 44 mg to about 46 mg, about 46 mg to about 48 mg, about 48 mg to about 50 mg, about 50 mg to about 200 mg, about 50 mg to about 70 mg, about 80 mg to about 100 mg, about 110 mg to about 130 mg, about 170 mg to about 190 mg, about 60 mg, about 90 mg, about 120 mg, or about 180 mg of dextromethorphan, or any amount of dextromethorphan in a range bounded by, or between, any of these values.

The amount of a compound of formula I, in a therapeutic composition may vary. If increasing the plasma level of dextromethorphan is desired, a compound of formula I should be administered in an amount that increases the plasma level of dextromethorphan. For example, a compound of formula I, may be administered in an amount that results in a plasma concentration of dextromethorphan in the subject, on day 8, that is at least about 2 times, at least about 5 times, at least about 10 times, at least about 15 times, at least about 20 times, at least about 30 times, at least about 40 times, at least about 50 times, at least about 60 times, at least about 70 times, or at least about 80 times, the plasma concentration of the same amount of dextromethorphan administered without a compound of formula I.

In some embodiments, a compound of formula I, may administered to a subject in an amount that results in a 12 hour area under the curve from the time of dosing (AUC₀₋₁₂), or average plasma concentration in the subject for the 12 hours following dosing (C_{avg}) of dextromethorphan, on day 8, that is at least about 2 times, at least about 5 times, at least about 10 times, at least about 15 times, at least about 20 times, at least about 30 times, at least about 40 times, at least about 50 times, at least about 60 times, at least about 70 times, or at least about 80 times the plasma concentration of the same amount of dextromethorphan administered without a compound of formula I.

In some embodiments, a compound of formula I, may administered to a subject in an amount that results in a maximum plasma concentration (Cₘₐₓ) of dextromethorphan in the subject, on day 8, that is at least about 2 times, at least about 5 times, at least about 10 times, at least about 15 times, at least about 20 times, at least about 30 times, or at least about 40 times the plasma concentration of the same amount of dextromethorphan administered without a compound of formula I.

For co-administration of a compound of formula I, an increase in the dextromethorphan plasma level can occur on the first day that a compound of formula I is administered, as compared to the same amount of dextromethorphan administered without a compound of formula I. For example, the dextromethorphan plasma level on the first day that a compound of formula I is administered may be at least about 1.5 times, at least about at least 2 times, at least about 2.5 times, at least about 3 times, at least about 4 times, at least about 5 times, at least about 6 times at least about 7 times, at least about 8 times, at least about 9 times, or at least about 10 times the level that would be achieved by administering the same amount of dextromethorphan without a compound of formula I.

In some embodiments, the dextromethorphan AUC on the first day that a compound of formula I is administered may be at least twice the AUC that would be achieved by administering the same amount of dextromethorphan without a compound of formula I.

In some embodiments, the dextromethorphan Cₘₐₓ on the first day that a compound of formula I is administered may be at least twice the Cₘₐₓ that would be achieved by administering the same amount of dextromethorphan without a compound of formula I.

In some embodiments, the dextromethorphan trough level (e.g., plasma level 12 hours after administration) on the first day that a compound of formula I is administered may be at least twice the trough level that would be achieved by administering the same amount of dextromethorphan without a compound of formula I.

In some embodiments, a compound of formula I is administered on the first day of at least two days of treatment with dextromethorphan, wherein a decrease in the DO plasma level occurs on the first day that a compound of formula I and dextromethorphan are co-administered, as compared to the same amount of dextromethorphan administered without a compound of formula I. For example, the DO plasma level on the first day may be reduced by at least 5% as compared to the DO plasma level that would be achieved by administering the same amount of dextromethorphan without a compound of formula I.

In some embodiments, a compound of formula I and dextromethorphan are co-administered for at least five consecutive days, to a subject in need of treatment with dextromethorphan, wherein, on the fifth day, the dextromethorphan plasma level is higher than the dextromethorphan plasma level that would have been achieved by administering the same amount of dextromethorphan administered without a compound of formula I, for five consecutive days. For example, the dextromethorphan plasma level on the fifth day (for example at 0 hours, 1 hour, 3 hours, 6 hours, or 12 hours after administration) may be at least 5 times, at least 10 times, at least 20 times, at least 40 times, at least 50 times, at least 60 times, at least 65 times, or up to about 500 times, the level that would be achieved by administering the same amount of dextromethorphan without a compound of formula I, for five consecutive days.

In some embodiments, a compound of formula I and dextromethorphan are co-administered for at least six consecutive days, to a subject in need of treatment with dextromethorphan, wherein, on the sixth day, the dextromethorphan plasma level is higher than the dextromethorphan plasma level that would have been achieved by administering the same amount of dextromethorphan administered without a compound of formula I, for six consecutive days. For example, the dextromethorphan plasma level on the sixth day (for example at 0 hours, 1 hour, 3 hours, 6 hours, or 12 hours after administration) may be at least 5 times, at least 10 times, at least 20 times, at least 30 times, at least 50 times, at least 60 times, at least 70 times, at least 75 times, or up to about 500 times, the level that would be achieved by administering the same amount of dextromethorphan without a compound of formula I, for six consecutive days.

In some embodiments, a compound of formula I and dextromethorphan are co-administered for at least seven consecutive days, to a subject in need of treatment with dextromethorphan, wherein, on the seventh day, the dextromethorphan plasma level is higher than the dextromethorphan plasma level that would have been achieved by administering the same amount of dextromethorphan administered without a compound of formula I, for seven consecutive days. For example, the dextromethorphan plasma level on the seventh day (for example at 0 hours, 1 hour, 3 hours, 6 hours, or 12 hours after administration) may be at least 5 times, at least 10 times, at least 20 times, at least 30 times, at least 50 times, at least 70 times, at least 80 times, at least 90 times, or up to about 500 times, the level that would be achieved by administering the same amount of dextromethorphan without a compound of formula I, for seven consecutive days.

In some embodiments, a compound of formula I and dextromethorphan are co-administered for at least eight consecutive days, wherein, on the eighth day, dextromethorphan has a plasma level, for example at 0 hours, 1 hour, 3 hours, 6 hours, or 12 hours, after co-administering a compound of formula I with dextromethorphan that is at least 5 times, at least 10 times, at least 20 times, at least 30 times, at least 50 times, at least 60 times, at least 70 times, at least 80 times, at least 90 times, at least 100 times, or up to about 1,000 times, the plasma level that would be achieved by administering the same amount of dextromethorphan without a compound of formula I for eight consecutive days.

In some embodiments, a compound of formula I and dextromethorphan are co-administered for at least eight consecutive days, to a subject in need of treatment with dextromethorphan, wherein, on the eighth day, the DO plasma level is lower than the DO plasma level that would have been achieved by administering the same amount of dextromethorphan administered without a compound of Formula I for eight consecutive days. For example, the DO plasma level on the eighth day (for example at 0 hours, 1 hour, 3 hours, 6 hours, or 12 hours after administration) may be reduced by at least 10%, at least 20%, at least 30%, at least 40%, or at least 50%, as compared to the DO plasma level that would be achieved by administering the same amount of dextromethorphan without a compound of formula I for eight consecutive days.

In some embodiments, a compound of formula I may be administered to a subject in an amount that results in an AUC₀₋₁₂ of a compound of formula I in the subject, on day 8, that is at least about 100 nghr/mL, at least about 200 nghr/mL, at least about 500 nghr/mL, at least about 600 nghr/mL, at least about 700 nghr/mL, at least about 800 nghr/mL, at least about 900 nghr/mL, at least about 1,000 nghr/mL, at least about 1,200 nghr/mL, at least 1,600 nghr/mL, or up to about 15,000 nghr/mL.

In some embodiments, a compound of formula I may be administered to a subject in an amount that results in a C_{avg} of a compound of formula I in the subject, on day 8, that is at least about 10 ng/mL, at least about 20 ng/mL, at least about 40 ng/mL, at least about 50 ng/mL, at least about 60 ng/mL, at least about 70 ng/mL, at least about 80 ng/mL, at least about 90 ng/mL, at least about 100 ng/mL, at least 120 ng/mL, or up to about 1,500 ng/mL.

Some liquid compositions may comprise about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of a compound of Formula I or any amount of a compound of Formula I, in a range bounded by, or between, any of these values.

Some liquid dosage forms may contain about 10 mg to about 1000 mg, about 50 mg to about 1000 mg, about 10 mg to about 50 mg, about 50 mg to about 100 mg, about 40 mg to about 90 mg, about 200 mg to about 300 mg, about 70 mg to about 95 mg, about 100 mg to about 200 mg, about 105 mg to about 200 mg, about 110 mg to about 140 mg, about 180 mg to about 220 mg, about 280 mg to about 320 mg, about 200 mg, about 150 mg, or about 300 mg of a compound of Formula I, or any amount of a compound of Formula I, in a range bounded by, or between, any of these values.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of a compound of Formula I, or any amount of a compound of Formula I, in a range bounded by, or between, any of these values.

Some solid dosage forms may contain about 10 mg to about 1000 mg, about 50 mg to about 1000 mg, about 10 mg to about 50 mg, about 50 mg to about 100 mg, about 40 mg to about 90 mg, about 200 mg to about 300 mg, about 70 mg to about 95 mg, about 100 mg to about 200 mg, about 105 mg to about 200 mg, about 110 mg to about 140 mg, about 50 mg to about 150 mg, about 180 mg to about 220 mg, about 280 mg to about 320 mg, about 200 mg, about 150 mg, or about 300 mg of a compound of Formula I, or any amount of a compound of Formula I, in a range bounded by, or between, any of these values.

In some embodiments, a compound of Formula I is administered at a dose that results in a a compound of Formula I, plasma level of about 0.1 μM to about 10 μM, about 0.1 μM to about 5 μM, about 0.2 μM to about 3 μM, 0.1 μM to about 1 μM, about 0.2 μM to about 2 μM, 1 μM to about 10 μM, about 1 μM to about 5 μM, about 2 μM to about 3 μM, or about 2.8 μM to about 3 μM, about 1.5 μM to about 2 μM, about 4.5 μM to about 5 μM, about 2.5 μM to about 3 μM, about 1.8 μM, about 4.8 μM, about 2.9 μM, about 2.8 μM, or any plasma level in a range bounded by, or between, any of these values.

In some embodiments, a compound of Formula I may be administered to a subject in an amount that results in an AUC₀₋₁₂ of a compound of Formula I in the subject, on day 8, that is at least about 200 nghr/mL, at least about 400 nghr/mL, at least about 700 nghr/mL, at least about 1,000 nghr/mL, at least about 3,000 nghr/mL, at least about 7,000 nghr/mL, at least about 10,000 nghr/mL, at least about 15,000 nghr/mL, at least about 20,000 nghr/mL, at least about 30,000 nghr/mL, up to about 50,000 nghr/mL, up to about 150,000 nghr/mL, or any AUC in a range bounded by, or between, any of these values.

In some embodiments, a compound of Formula I is administered to a subject in an amount that results in a Cₘₐₓ of a compound of Formula I in the subject, on day 8, that is at least about 20 ng/mL, at least about 60 ng/mL, at least about 90 ng/mL, at least about 100 ng/mL, at least about 150 ng/mL, at least about 200 ng/mL, at least about 300 ng/mL, up to about 1,000 ng/mL, at least about 4,000 ng/mL, up to about 10,000 ng/mL, up to about 50,000 ng/mL, or any Cₘₐₓ in a range bounded by, or between, any of these values.

In some embodiments, a compound of Formula I is administered to a subject in an amount that results in a C_{avg} of a compound of Formula I in the subject, on day 8, that is at least about 20 ng/mL, at least about 30 ng/mL, at least about 50 ng/mL, at least about 80 ng/mL, at least about 90 ng/mL, at least about 100 ng/mL, at least about 150 ng/mL, at least about 200 ng/mL, at least about 300 ng/mL, up to about 1,000 ng/mL, up to about 5,000 ng/mL, up to about 30,000 ng/mL, or any Cₐvg in a range bounded by, or between, any of these values.

For compositions comprising both dextromethorphan and a compound of Formula I some liquids may comprise about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), about 40% (w/v) to about 50% (w/v) of dextromethorphan and a compound of Formula I combined, or any amount in a range bounded by, or between, any of these values. Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), about 80% (w/w) to about 90% (w/w) of dextromethorphan and a compound of Formula I combined, or any amount in a range bounded by, or between, any of these values. In some embodiments, the weight ratio of dextromethorphan to a compound of Formula I in a single composition or dosage form may be about 0.1 to about 2, about 0.2 to about 1, about 0.1 to about 0.3, about 0.2 to about 0.4, about 0.3 to about 0.5, about 0.5 to about 0.7, about 0.8 to about 1, about 0.2, about 0.3, about 0.4, about 0.45, about 0.6, about 0.9, or any ratio in a range bounded by, or between, any of these values.

A therapeutically effective amount of a therapeutic compound may vary depending upon the circumstances. For example, a daily dose of dextromethorphan may in some instances range from about 0.1 mg to about 1000 mg, about 40 mg to about 1000 mg, about 20 mg to about 600 mg, about 60 mg to about 700 mg, about 100 mg to about 400 mg, about 15 mg to about 20 mg, about 20 mg to about 25 mg, about 25 mg to about 30 mg, about 30 mg to about 35 mg, about 35 mg to about 40 mg, about 40 mg to about 45 mg, about 45 mg to about 50 mg, about 50 mg to about 55 mg, about 55 mg to about 60 mg, about 20 mg to about 60 mg, about 60 mg to about 100 mg, about 100 mg to about 200 mg, about 100 mg to about 140 mg, about 160 mg to about 200 mg, about 200 mg to about 300 mg, about 220 mg to about 260 mg, about 300 mg to about 400 mg, about 340 mg to about 380 mg, about 400 mg to about 500 mg, about 500 mg to about 600 mg, about 15 mg, about 30 mg, about 60 mg, about 120 mg, about 180 mg, about 240 mg, about 360 mg, or any daily dose in a range bounded by, or between, any of these values. dextromethorphan may be administered once daily; or twice daily or every 12 hours, three times daily, four times daily, or six times daily in an amount that is about half, one-third, one-quarter, or one-sixth, respectively, of the daily dose.

A daily dose of a compound of Formula I may in some instances range from about 10 mg to about 1000 mg, about 50 mg to about 600 mg, about 100 mg to about 2000 mg, about 50 mg to about 100 mg, about 70 mg to about 95 mg, about 100 mg to about 200 mg, about 105 mg to about 200 mg, about 100 mg to about 150 mg, about 150 mg to about 300 mg, about 150 mg to about 200 mg, about 200 mg to about 250 mg, about 250 mg to about 300 mg, about 200 mg about 300 mg, about 300 mg to about 400 mg, about 400 mg to about 500 mg, about 400 mg to about 600 mg, about 360 mg to about 440 mg, about 560 mg to about 640 mg, or about 500 mg to about 600 mg, about 100 mg, about 150 mg, about 200 mg, about 300 mg, about 400 mg, about 600 mg, or any daily dose in a range bounded by, or between, any of these values. A compound of Formula I may be administered once daily; or twice daily or every 12 hours, or three times daily in an amount that is about half or one-third, respectively, of the daily dose.

In some embodiments: 1) about 50 mg/day to about 100 mg/day, about 100 mg/day to about 150 mg/day, about 150 mg/day to about 300 mg/day, about 150 mg/day to about 200 mg/day, about 200 mg/day to about 250 mg/day, about 250 mg/day to about 300 mg/day of a compound of Formula I or about 300 mg/day to about 500 mg/day of a compound of Formula I; and/or 2) about 15 mg/day to about 60 mg/day, about 15 mg/day to about 30 mg/day, about 30 mg/day to about 45 mg/day, about 45 mg/day to about 60 mg/day, about 60 mg/day to about 100 mg/day, about 80 mg/day to about 110 mg/day, about 100 mg/day to about 150 mg/day, or about 100 mg/day to about 300 mg/day of dextromethorphan, are administered to a subject in need thereof.

In some embodiments, about 150 mg/day of a compound of Formula I and about 30 mg/day of dextromethorphan, about 150 mg/day of a compound of Formula I and about 60 mg/day of dextromethorphan, about 150 mg/day of a compound of Formula I and about 90 mg/day of dextromethorphan, about 150 mg/day of a compound of Formula I and about 120 mg/day of dextromethorphan, about 200 mg/day of a compound of Formula I and about 30 mg/day of dextromethorphan, about 200 mg/day of a compound of Formula I and about 60 mg/day of dextromethorphan, about 200 mg/day of a compound of Formula I and about 90 mg/day of dextromethorphan, about 200 mg/day of a compound of Formula I and about 120 mg/day of dextromethorphan, about 300 mg/day of a compound of Formula I and about 30 mg/day of dextromethorphan, about 300 mg/day of a compound of Formula I and about 60 mg/day of dextromethorphan, about 300 mg/day of a compound of Formula I and about 90 mg/day of dextromethorphan, or about 300 mg/day of a compound of Formula I and about 120 mg/day of dextromethorphan is administered to the subject.

In some embodiments, about 100 mg/day of a compound of Formula I and about 15 mg/day of dextromethorphan is administered to the subject for 1, 2, or 3 days, followed by about 200 mg/day of a compound of Formula I and about 30 mg/day of dextromethorphan. In some embodiments, about 100 mg/day of a compound of Formula I and about 30 mg/day of dextromethorphan is administered to the subject for 1, 2, or 3 days, followed by about 200 mg/day of a compound of Formula I and about 60 mg/day of dextromethorphan.

In some embodiments, about 75 mg/day of a compound of Formula I and about 15 mg/day of dextromethorphan is administered to the subject for 1, 2, or 3 days, followed by about 150 mg/day of a compound of Formula I and about 30 mg/day of dextromethorphan. In some embodiments, about 75 mg/day of a compound of Formula I and about 30 mg/day of dextromethorphan is administered to the subject for 1, 2, or 3 days, followed by about 150 mg/day of a compound of Formula I and about 60 mg/day of dextromethorphan.

Therapeutic compounds may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, troches, capsules, elixirs, suspensions, syrups, wafers, and the like.

Tablets, troches, pills, capsules and the like may also contain one or more of the following: a binder such as gum tragacanth, acacia, cornstarch, or gelatin; an excipient, such as dicalcium phosphate; a disintegrating agent such as corn starch, potato starch, alginic acid, and the like; a lubricant such as magnesium stearate; a sweetening agent such as sucrose, lactose, or saccharin; or a flavoring agent such as peppermint, oil of wintergreen, or cherry flavoring. When the dosage unit form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially nontoxic in the amounts employed.

Some compositions or dosage forms may be a liquid or may comprise a solid phase dispersed in a liquid. Therapeutic compounds may be formulated for parenteral or oral administration. Solutions of the active compounds as free bases or pharmacologically acceptable salts can be prepared in water suitably mixed with a surfactant, such as hydroxypropylcellulose. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

Although dementias such as Alzheimer's disease (AD) are characterized by cognitive deficits, neuropsychiatric symptoms (behavioral and psychological symptoms of dementia, BPSD) are among the main drivers for caregiver burden and hospitalization. Frequency of BPSD symptoms increases with the disease progression (e.g. up to 60% in mild and moderate AD and up to 90% in severe AD).

Currently marketed dementia therapies leave much room for improvement when it comes to treat BPSD but also other non-cognitive areas of concern. In the continued absence of a disease-modifying therapy, this is of increasing importance, as symptoms like hostility, aggression, wandering, sexually inappropriate behavior or incontinence pose major problems to caregivers and families, and are predictors for (costly) nursing home placement.

It is common global practice to prescribe (typical or atypical) neuroleptics to facilitate nursing and caregiving. However, the FDA has determined that off-label prescription of neuroleptics poses a major threat to the health of demented subjects, and has issued a black box warning, citing severe cardiovascular adverse events and an increased risk for death. EU approval of risperidone allows for short-term use in moderate-severe AD patients only in case of harm to self or others. In Parkinson's Disease, the anticholinergic effects of neuroleptics are highly unwanted as they inevitably worsen, in addition, the motor condition and symptoms of the vegetative nervous system. In all dementias, lowering the seizure threshold is another infrequent but highly unwanted potential adverse effect of neuroleptics. These concerns about the use of neuroleptic drugs in dementias result in decreased use of neuroleptics in this category of patients leaving BPSD symptoms in the vast majority of mild-to-moderate AD patients essentially untreated.

Accordingly, several embodiments are novel compositions and methods useful in the symptomatic and disease-modifying treatment of neurodegenerative diseases and brain injuries including sequelae thereof like organic brain syndrome and chronic traumatic encephalopathies; chronic or intractable pain, ophthalmologic indications associated with retinopathies, anxiety disorders, post-traumatic stress disorder, depression, diabetes mellitus and it's complications like peripheral neuropathies with or without neuropathic pain, Buerger's disease, Raynaud's disease, coronary artery disease, angina pectoris, atherosclerosis including CNS like multi-infarct dementia, Vascular Cognitive Impairment, Vascular Dementia or Binswanger's Disease, and nephropathies.

In an embodiment of the fifth aspect, dextromethorphan is administered in a combined dose with a selected enantiomer of Formula I, wherein the amount of the compound of Formula I enantiomer administered comprises from about 0.1 mg/day to about 1000 mg/day.

Some embodiments include a method of treating a disease or disorder comprising administering about 5 mg/day to about 600 mg/day, about 5 mg/day to about 300 mg/day, about 5 mg/day to about 400 mg/day, about 5 mg/day to about 500 mg/day, about 5 mg/day to about 600 mg/day, about 5 mg/day to about 1,000 mg/day, about 50 mg/day to about 1000 mg/day, about 100 mg/day to about 1000 mg/day, about 150 mg/day to about 1000 mg/day, about 150 mg/day to about 5000 mg/day, about 150 mg/day to about 300 mg/day, or about 150 mg/day to about 100 mg/day, or an amount as required of a compound of Formula I and about 0.1 mg/day to about 1 mg/day, about 0.5 mg/day to about 15 mg/day, about 15 mg/day to about 60 mg/day, about 15 mg/day to about 120 mg/day, about 0.1 mg/day to about 200 mg/day, or any amount of a compound of Formula I in a range bounded by, or between, any of these values, or an amount as required of dextromethorphan to a subject in need thereof.

The composition of the present invention can be formulated into any pharmaceutical dosage forms for oral, topical, rectal, vaginal, nasal, or ophthalmic administration, and include. syrups and suspensions, using commonly known ingredients and procedures and methods can be used to formulate the compositions of the invention.

The present invention can be formulated into any pharmaceutical dosage forms for oral, topical, rectal, vaginal, nasal, or ophthalmic administration, and include. syrups and suspensions, and commonly known ingredients and procedures to formulate pharmaceutical composition are within the purview of a person skilled in the art, including various known methods can be used to formulate the composition of the invention.

The oral formulations and the tablet formulations include enteric coating layered formulations that comprise a separating layer to separate the acidic enteric coating material from omeprazole being an acid susceptible substance. HPC or other suitable polymers disclosed herein may be used in a layer that separates the core material from the enteric coating layer in the described formulations.

### Synthetic Methods

The biosynthesis of CBD involves many steps, and begins with the formation of cannabigerolic acid (CBGA), which, after exposure to CBGA synthase becomes cannabidiolic acid (CBDA). Either through exposure to heat, or over large periods of time, a carboxylic acid group is removed from CBDA to yield CBD. The quantity of CBD produced is contingent upon the amount of CBGA and the activity of CBDA synthase in a cannabis plant, as well as the amount of heat applied to CBDA. Heat may be derived through combustion (as in smoking), or through a vaporizer. In the case of concentrated oils, CBDA is converted to CBD in industrial heated ovens. CBD can also be created synthetically, although this is not a common method of production.

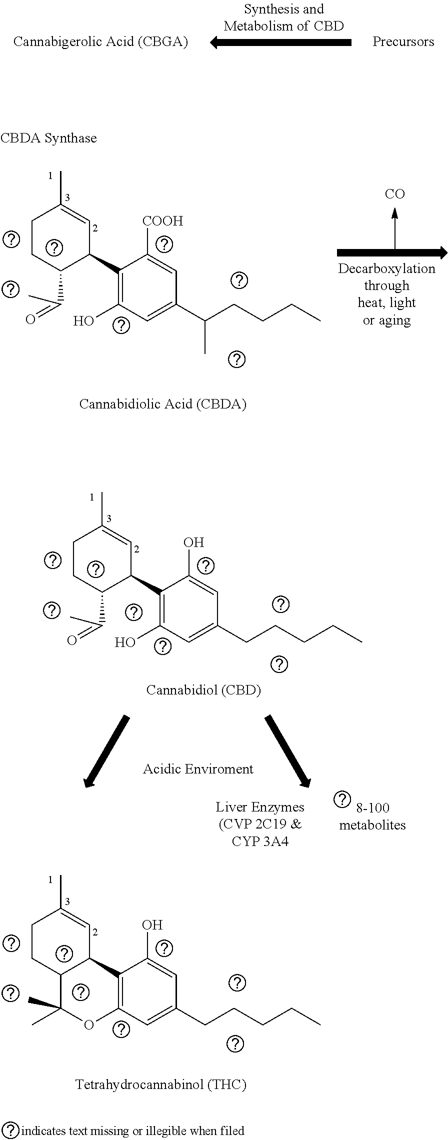

Various versatile and convenient chiral carboxylic acid ligands are available in the literature such as mandelic acid, 2-methylmandelic acid, 2-chloromandelic acid, 3-chloromandelic acid, 4-methoxymandelic acid, O-acetylmandelic acid, α-methoxyphenylacetic acid, malic acid, tartaric acid, etc. The chiral ligands can be prepared from readily available building blocks.

Formation of the diastereomeric compounds and salts is carried out in a suitable reaction medium. Suitable reaction media include water, methanol, ethanol, 1-propanol, 2-propanol, diethyl ether, methyl tert-butyl ether, tetrahydrofuran, acetic acid, methyl acetate, ethyl acetate, isopropyl acetate, butyl acetate, acetone, methyl ethyl ketone, methyl isobutyl ketone, acetonitrile, methylene chloride, chloroform, 1,2-dichloroethane, benzene, toluene and xylenes, and/or mixtures thereof.

Esterification Esters are derived from carboxylic acids. A carboxylic acid contains the —COOH group, and in an ester the hydrogen in this group is replaced by a hydrocarbon group R′ such as an alkyl, cycloalkyl, an aryl, and a hetero-aryl group. Esters are produced when carboxylic acids are heated with alcohols in the presence of an acid catalyst. The catalyst is an acid, usually concentrated sulfuric acid. Dry hydrogen chloride gas can be used in some cases. TsOH (tosic acid) is also often used.

The esterification reaction is both slow and reversible. The equation for the reaction between an acid RCOOH and an alcohol R′OH (where R and R′ can be the same or different) is:

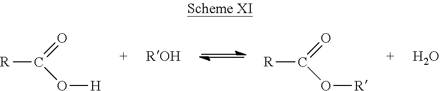

### EXAMPLES

### Optically Pure Dextromethorphan and Formula I

Compound of formula I and dextromethorphan salt in chloroform or other suitable solvents such as dichloromethane, DMF, etc., which can be separated by crystallization and recrystallization in suitable solvents such DMF and/or chromatographic techniques referred to and described in this specification.

In one embodiment, provided is a process for separating the diastereomers of a compound by using an ionic liquid to increase separation efficiency. When the diastereomers are separated, for example, by a process such as liquid-liquid extraction, one or more ionic liquids may be used as the extractant.

In one embodiment, this separation process may be performed on a compound containing a mixture of at least one pair of diastereomers, and the diastereomers may be separated by contacting the mixture with at least one ionic liquid in which one of the diastereomers is soluble to a greater extent than the other diastereomer, and separating the lower-solubility diastereomer from the mixture. The inventions disclosed herein thus include processes for the separation of diastereomers, the use of such processes, and the products obtained and obtainable by such processes.

In another embodiment, this separation process may be performed on a compound such as a diastereomeric mixture of salt wherein, the diastereomers are separated by contacting the mixture with at least one ionic liquid in which one of the diastereomers is soluble to a greater extent than the other diastereomer, and separating the lower-solubility diastereomer from the mixture.

In yet another embodiment, there is provided a process for separating the erythro or threo diastereomers from a mixture comprising both diastereomers by liquid-liquid extraction using at least one ionic liquid as an extractive solvent.

Another embodiment is a process for performing an industrial operation selected from the group consisting of a calibration operation, a cleaning operation, a rinsing operation, a drying operation, a particulate removal operation, a solvent operation, a dispersion operation, a heat transfer operation, and an insulating operation, comprising contacting a mixture comprising a pair of diastereomers with at least one ionic liquid in which one of the diastereomers is soluble to a greater extent than the other diastereomer, separating the lower-solubility diastereomer from the mixture, and employing the separated diastereomer in the operation.

Another embodiment is a process for separating one diastereomer from another diastereomer in a pair of diastereomers in a compound. In such a process, an ionic liquid is used to facilitate the separation, and the diastereomers may be separated by contacting the mixture with at least one ionic liquid in which one of the diastereomers is soluble to a greater extent than the other diastereomer, and separating the lower-solubility diastereomer from the mixture.

The term “ionic liquid” is defined as an organic salt that is fluid at or below about 100° C.

“Liquid-liquid extraction” is a process for separating components in solution by their distribution between two immiscible liquid phases. Liquid-liquid extraction involves the transfer of mass from one liquid phase into a second immiscible liquid phase, and is carried out using an extractant or solvent.

Components in a liquid mixture can be separated by a process such as liquid-liquid extraction using a single equilibrium (or theoretical) stage, or using multiple stages. An equilibrium, or theoretical, stage is a device that allows intimate mixing of a feed with an immiscible liquid such that concentrations approach equilibrium, followed by physical separation of the two immiscible liquid phases. A single stage device can be a separatory funnel, or an agitated vessel, which allows for intimate mixing of the feed with the immiscible extractant. Following intimate mixing, one or both of the liquid phases can be recovered, for example, by decantation.

Multiple stage devices for liquid separation can be crosscurrent or countercurrent devices. In a multiple stage device, the feed enters a first equilibrium stage and is contacted with an extractant. The two liquid phases are mixed, with droplets of one phase suspended in the second phase, and then the two phases are separated, from the first stage is contacted with additional extractant, and the separation process is repeated. The process of (1) contacting with extractant, (2) allowing for equilibrium concentrations to be approached, and (3) separating the liquid phases is repeated until the desired purity of the component of interest is achieved. The number of equilibrium stages will depend on the desired purity, as well as the solubility of the components in the extractant and the flow rates of the feed and extractant.

In a crosscurrent system (or device), the feed is initially contacted with extractant in a first equilibrium stage. Dextromethorphan and Formula I, from this stage then cascades down through one or more additional stages. At each stage, the composition is contacted with fresh extractant, and further purification of the desired component in the composition is achieved. An example of a crosscurrent system where the threo isomer of the composition is purified using the ionic liquid 1-butyl-3-methylimidazolium tetrafluoroborate ([BMIM][BF₄] as the extractant. In a countercurrent system or device, the extractant enters at the stage farthest from the feed, and the two phases are passed through and across each other, coming from the two different (e.g. opposite) directions.

Equipment used for liquid-liquid extraction can be classified as “stagewise” or “continuous (differential) contact” equipment. Stagewise equipment is also referred to as “mixer-settlers”. Mixing the liquids occurs by contacting the feed with the extractant, and the resultant dispersion is settled as the two phases separate. Mixing can occur with the use of baffles or impellers, and the separation process may be carried out in batch fashion or with continuous flow. Settlers can be simple gravity settlers, such as decanters, or can be cyclones or centrifuges, which enhance the rate of settling.

Continuous contact equipment is typically arranged for multistage countercurrent contact of the immiscible liquids, without repeated separation of the liquids from each other between stages. Instead, the liquids remain in continuous contact throughout their passage through the equipment. Countercurrent flow is maintained by the difference in densities of the liquids and either the force of gravity (vertical towers) or centrifugal force (centrifugal extractors). Gravity-operated extractors can be classified as spray towers, packed towers or perforated-plate (sieve-plate) towers. Gravity-operated towers also include towers with rotating stirrers and pulsed towers as is known in the art.

When the diastereomers of a compound of the composition, and in particular the threo and erythro isomers of 2,3-dihydrodecafluoropentane, are separated by a process such as liquid-liquid extraction, any of the equipment described above can be used to perform the separation. In one preferred embodiment, the separation is carried out using a vertical tower with perforated plates. After separation of the phase containing the lower-solubility diastereomer from the phase containing the extractant and the higher-solubility diastereomer, the higher solubility diastereomer may be separated from the extractant by a process such as distillation.

The dielectrical constant of the solvent (if solvent is used at the resolutions) changes the formation, composition and enantiomer recognition of the crystalls. The composition of crystalline diastereoisomers is also influenced by the pH of the reaction mixture. The purity (de) of the diastereoisomer can be improved using a mixture of structurally related resolving agents. It is often referred as “Dutch resolution.” If the diastereoisomeric salt cannot be separated by fractionated precipitation, it is feasible to get its crystalline solvate by fractionated precipitation from a solvate forming solution. When the solvent, unsuitable for separation of the diastereoisomers, contains structurally partly similar compounds to the solvate forming solution, the separation of enantiomers became feasiable by fractionated precipitation of the diastereoisomeric salt.

At the crystallization of melts of racemate forming enantiomeric mixtures the eutectic composition usually determinates the composition of the crystallized mixture and the oily residue. That eutectic composition can be known from the binary melting point phase diagram. When the initial isomeric composition (ee0) is higher than the eutectic composition, the pure optical isomer cam be crystallized.

An ionic liquid, or a mixture of two or more thereof, may be used in a process hereof to separate the diastereomers of a compound. When, for example, the diastereomers are separated by a process such as liquid-liquid extraction, the extractant used may be an ionic liquid or a mixture of two or more ionic liquids. Ionic liquids are organic compounds that are liquid at room temperature (approximately 25° C.). They differ from most salts in that they have very low melting points, and they generally tend to be liquid over a wide temperature range. They also generally tend to not be soluble in non-polar hydrocarbons; to be immiscible with water (depending on the anion); and to be highly ionizing (but have a low dielectric strength). Ionic liquids have essentially no vapor pressure, most are air and water stable, and they can either be neutral, acidic or basic.

A cation or anion of an ionic liquid useful herein can in principle be any cation or anion such that the cation and anion together form an organic salt that is liquid at or below about 100° C. The properties of an ionic liquid can, however, be tailored by varying the identity of the cation and/or anion. For example, the acidity of an ionic liquid can be adjusted by varying the molar equivalents and type and combinations of Lewis acids used.

Many ionic liquids are formed by reacting a nitrogen-containing heterocyclic ring, preferably a heteroaromatic ring, with an alkylating agent (for example, an alkyl halide) to form a quaternary ammonium salt, and performing ion exchange or other suitable reactions with various Lewis acids or their conjugate bases to form the ionic liquid. Examples of suitable heteroaromatic rings include substituted pyridines, imidazole, substituted imidazole, pyrrole and substituted pyrroles. These rings can be alkylated with virtually any straight, branched or cyclic C₁₋₂₀ alkyl group, but preferably, the alkyl groups are C₁₋₁₆ groups, since groups larger than this may produce low melting solids rather than ionic liquids. Various triarylphosphines, thioethers and cyclic and non-cyclic quaternary ammonium salts may also been used for this purpose. Counter ions that may be used include chloroaluminate, bromoaluminate, gallium chloride, tetrafluoroborate, tetrachloroborate, hexafluorophosphate, nitrate, trifluoromethane sulfonate, methylsulfonate, p-toluenesulfonate, hexafluoroantimonate, hexafluoroarsenate, tetrachloroaluminate, tetrabromoaluminate, perchlorate, hydroxide anion, copper dichloride anion, iron trichloride anion, zinc trichloride anion, as well as various lanthanum, potassium, lithium, nickel, cobalt, manganese, and other metal-containing anions.

Ionic liquids may also be synthesized by salt metathesis, by an acid-base neutralization reaction or by quaternizing a selected nitrogen-containing compound; or they may be obtained commercially from several companies such as Merck (Darmstadt, Germany) or BASF (Mount Olive, N.J.).

In one embodiment, a library of ionic liquids may be prepared, for example, by preparing various alkyl derivatives of a particular cation (such as the quaternary ammonium cation), and varying the associated anions. In another embodiment, the diastereomers of the invention can be separated efficiently by cation exchange with mixed-mode sorbent in the solid phase extraction (SPE) procedure.

In one embodiment, diastereomers can be separated by extractive distillation, wherein an auxiliary which changes the partial pressure of the various diastereomers to be separated to a different degree allowing easier separation of the diastereomers by distillation in a good yield. Separation can be accomplished using fractionating columns, and preferably under reduced pressure of about 10⁻³ bar to about 1 bar.

In one embodiment, reversed (RP-HPLC) and normal phase chromatographic (NP-HPLC) separations can be used to separate the diastereomers of the invention Columns that can be used in the separation of enantiomers can be Primesep C, NUCLEOSI, cellulose based chiral IPLC columns, SHISEIDO Chiral CD-Ph, etc.

### Pharmaceutical Formulations

The compositions of this invention can be prepared by adding a compound of Formula I, and dissolved in, a suitable solvent. The solution, thus obtained, is added to the complex magnesium aluminum silicate to form a paste-like mass. While the foregoing steps are carried out at about room temperature, elevated temperatures can be employed if desired. Subsequently, sodium chloride and sodium saccharin are added to, and uniformly distributed throughout, the paste. Edible coloring and flavoring materials can be incorporated into the system at any stage of the ipreparative method. In another embodiment, soluble ingredients are added to a compound of Formula I solution which is prepared in the first step. The paste which is thus obtained can be incorporated readily into a conventional hard candy-forming mass, which mass, in turn, can be worked up, by conventional procedures, into attractive, pleasant-tasting lozenges each containing therapeutically effective quantities of a compound of Formula I uniformly distributed throughout.

Variations in the preparative methods presented here are within the scope of the present invention. For example, in producing the compositions of the invention, one can mix racemate or enatiomerically pure compound of Formula I and the complex magnesium aluminum silicate and subsequently add a suitable solvent thereto to form a paste therewith. Sodium chloride and sodium saccharin can be added to the dextromethorphan-complex magnesium aluminum silicate mixture prior to forming the mixture into a paste. In the alternative, sodium chloride and sodium saccharin can be added to the paste. Furthermore, suitable flavoring agents and coloring agents can be added either to the dry mixture or to the paste. In carrying out this invention, any medicinally acceptable organic solvent which is suitable for pharmaceutical use and in which a compound of Formula I soluble can be employed. Thus, for example, organic solvents, such as propylene glycol, glycerine, 1,3-butylene glycol, benzyl alcohol, etc., can be used. In an embodiment of compositions of the invention, benzyl alcohol is employed as the solvent.

Edible coloring agents and edible flavoring agents can be used in preparing the present compositions. Flavoring agents which are suitable for use include, for example, licorice, ginger, natural fruit extracts, etc. As the coloring agent one can use any color which is suitable for use in foods and drugs. The quantity of coloring and the quantity of flavoring agents used in formulating the composition of this invention is variable.

In an embodiment, the formulation contains about 0.3 g to about 1.5 g, about 1.0 g, of thickener; about 1 g to about 10 g, about 2.5 g, of 1,2-propylen glycol as a dissolving agent; about 0.12 g to about 0.19 g, or 0.15 g, of at least one paraben preservative such as methyl paraben; about 0.05 g to about 0.2 g, or about 0.1 g, of sorbic acid; about 30 g to about 60 g, or 40 g of a sugar alcohol solution; about 0.05 to about 0.2 g, or 0.1 g of an artificial sweetener; a compound of Formula I resin complex in an amount to yield a desired strength of about 2.10 g (the amount of a 1:6 complex needed to deliver equivalent to 60 mg of a compound of Formula I in a 20 ml adult 12 hour dose); and sufficient water to bring the volume up to 100 ml.

In another embodiment, suitable thickeners include: tragacanth; bentonite; acacia and lower alkyl ethers of cellulose (including the hydroxy and carboxy derivatives of the cellulose ethers). Exemplary paraben preservatives are C1-C4 alkyl parabens namely methyl, ethyl, propyl, and butyl parabens. In one embodiment, both methyl and propyl paraben are present in the formulation in a ratio of methyl paraben to propyl paraben of from about 2.5:1 to about 7.5:1. In another embodiment the methyl and propyl paraben ratio is 4:1.

In one embodiment, the artificial sweetener is a form of saccharin or aspartame. In one embodiment, saccharin is sacharin sodium. In other embodiments, equivalent sweetening amounts of other known sweetening agents such as the sugar alcohol sorbitol may be substituted therefor.

In another embodiment, the formulation comprises an amount of resinate sufficient to deliver, when administered at one dose every 12 hours, an antitussive effective amount of a compound of Formula I over a period of approximately 12 hours to a patient in need of such administration.

In an embodiment, the formulation comprises an adult dose of 20 ml contains approximately 420 mg of resinate, to deliver equivalent to 60 mg of a compound of Formula I when the drug to resin ratio is 1:6 and 2.10 g of resinate are present per 100 ml of formulation. The dosage can be altered analogously to that known for the administration of dextromethorphan which has not been complexed with resin, i.e. the typical 15 mg-30 mg/dose of dextromethorphan hydrobromide 1 to 4 times daily, becomes S-20 ml once to twice daily.

In another embodiment, the formulation comprises the nontoxic substances that block the NMDA receptor in accordance with this invention are dextromethorphan ((+)-3-hydroxy-Nmethylmorphinan), a compound of Formula I or derivatives thereof, and Saprodexter™, mixtures and pharmaceutically acceptable salts thereof.

In another embodiment, the formulation comprises substances that block the NMDA receptor include dizocilpine (5-iethyl-10,11-dihydro-5H-5,10-epimninodibenzo[a,d][7]annulene), ketamine (2(2-chlorophenyl)-2-(methylamino)cyclohexan-1-one), magnesiurn, selfotel ((2S,4R)-4-(phosphonomethyl)piperidine-2-carboxylic acid), aptiganel ((E)-1-(3-ethylphenyl)-1-methyl-2-(naphthalen-1-yl)guanidine), felbamate (2-phenylpropane-1,3-diyl dicarbamate), phencyclidine (1-(1-phenylcyclohexyl)piperidine), amantadine (1-aminoadamantine), memantine (3,5 dimethylaminoadamantone), pyrroloquinoline quinone (PQQ, 4,5-dioxo-4,5-dihydro-1H-pyrrolo[2,3-f]quinoline-2,7,9-tricarboxylic acid), (R)-(E)-4-(3-phosphonoprop-2-enyl)piperazine-2-carboxylic acid, (R)-2-amino-5-phosphonopentanoate, (S) and (R) 6-(1HTetrazol-5-ylmethyl)decahydroisoquinoline-3-carboxylic acid, (S)-a-amino-5-(phosphonomethyl)[1,19-biphenyl]-3-propanoic acid, (S) and (R) (6)-cis-4-(4-phenylbenzoyl) piperazine-2,3-dicarboxylic acid, cis-4-phosphonomethyl-2-piperidine carboxylic acid, 2R,4R,5S-(2-amino-4,5-(1,2-cyclohexyl)-7-phosphonoheptanoic acid), and cis-4-(phosphonomethyl)-2-piperidinecarboxylic acid, mixtures and pharmaceutically acceptable salts thereof.

In another embodiment, the therapeutic composition comprises at least one other pharmacologically active substance e.g., caffeine (a stimulant), an antiemetic drug such as metoclopramide, domperidone, belladonna alkaloids and phenothiazines such as chlorpromazine, prochlorperazine, and promethazine, a nonnarcotic analgesic, e.g., acetaminophen or a nonsteroidal anti-inflammatory drug such as aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and the like.

### Synthesis of Compounds of the Invention

All reactions were performed under an argon atmosphere with dry solvents, unless otherwise stated. Dry chloroform (CH₃Cl), methylene chloride (CH₂Cl₂), tetrahydrofuran (THF), ethyl acetate, DMF, DMSO, methanol, ethanol, and acetonitrile (CH₃CN) were purchased or prepared. All commercially available reagents were purchased and used without further purification. Reactions were monitored by thin-layer chromatography (TLC) on silica gel plates (Merck TLC Silica Gel 60 F254) using UV light, PMA (an ethanolic solution of phosphomolybdic acid) or ANIS (an ethanolic solution of para-anisaldehyde) as visualizing agent. Purification of products was conducted by column chromatography through silica gel 60 (0.060-0.200 mm). NMR spectra were obtained on Bruker AVANCE III 500 MHz (Bruker Corporation, Billerica, Mass., USA) using residual undeuterated solvent or TMS (tetramethylsilane) as an internal reference. High-resolution mass spectra (HR-MS) were recorded on a JEOL JMS-700 (JEOL, Tokyo, Japan) using EI (electron impact).

### Example 1

Dextromethorphan has been synthesized from a benzylisoquinoline (with a planar structure) by Grewe's cyclization to give the corresponding morphinan, wherein the 1,2,3,4,5,6,7,8-octahydro-1-(4-methoxybenzyl)isoquinoline is converted into the N-formyl derivative, cyclized to the N-formyl normorphinan, and the formyl group reduced to an N-methyl group, to give 3-methoxy-17-methylmorphinan. Dextromethorphan is freely soluble in ethanol 96% and essentially insoluble in water. Dextromethorphan can be monohydrated hydrobromide salt or bound to an ion exchange resin based on polystyrene sulfonic acid. Dextrometorphan's specific rotation in water is +27.6° (20° C., Sodium D-line).

### Example 2

Equimolar compound of formula I (429.506 g/mol) and dextromethorphan (271.40 g/mol) were mixed in a suitable solvent, agitated and let crystallize. The compound of Formula I and dextromethorphan positive cation would form hydrogen bond to form a complex and crystallize.

### Example 3

To a solution of 54.28 g of dextromethorphan in one liter of chloroform is added a solution of 85.9 g of cannabidiol in chloroform at 70° C. The salt is precipitated from the hot solution by the addition of ethyl acetate. After cooling the salt is collected, washed with ethyl acetate and dried to yield d-3-methoxy-N-methylmorphinan 4-[1-dimethylamino-3-[2-[2-(3-methoxyphenyl) ethyl]phenoxy]propan-2-yl] oxy-4-oxobutanoate salt and recrystallized from aqueous dimethylformamide (DMF) to yield of 135 g of the compound of Formula I.

### Example 4

Ingredients: 15 g of a compound of Formula I, 15 g Glyceryl tristearate; 100 ml Carbon tetrachloride. Preparation: Glyceryl tristearate is dissolved in the warm carbon tetrachloride at 55-60° C. A compound of formula I, derivative thereof, is then added and suspended in the solution. The suspension is then spray dried using an inlet temperature of 90° C. and an outlet temperature of 40° C. The resulting coated a compound of Formula I having an average particle size of from about 10 to about 200 microns is then suspended in the following aqueous vehicle.

Ingredients: 10.00 g Tragacanth, USP; 1.20 g Methylparaben, USP; 0.20 g Propylparaben, USP; 0.30 g Saccharin sodium, USP; 3.00 g Sucaryl sodium, USP; 250.00 mL Sorbic acid; 1.00 g Methyl cellulose, 15 cps; 2.00 mL Imitation black currant; and 1000.00 mL Distilled water.

The parabens, saccharin sodium, sucaryl sodium and sorbic acid are dissolved in a portion of the distilled water which has been heated to 85° C. The tragacanth is then added to this solution and dispersed uniformly. The dispersion is again heated, cooled and the sorbitol solution, a solution of the methyl cellulose in water and the imitation black currant are then added with mixing to form the vehicle. The coated a compound of Formula I is then added to the above vehicle and mixed until the particles are thoroughly wetted and uniformly dispersed.

The controlled drug-release composition of the present invention is characterized by comprising 100 parts by weight of an organic polymeric material which is soluble in an organic solvent and insoluble in water; 5 to 60 parts by weight of a lipid-soluble, low molecular weight release auxiliary agent; and 1 to 70 parts by weight of a drug.

In one embodiment, the polymeric material is biodegradable or biocompatible, or both, for example, biodegradable aliphatic polyester, or an aliphatic poly(carbonate), poly(lactic acid), lactic acid-glycolic acid copolymer, poly(caprolactone), poly(hydroxybutyric acid) and the like.

In one embodiment, the release auxiliary agent is a carboxylic acid ester, a monoester or diester of glycerin. In another embodiment, the release auxiliary agent is an ester of an organic acid selected from succinic acid, citric acid, tartaric acid, malic acid or the like, or monoacetate ester or diacetate ester of glycerin.

In one embodiment, the composition may further comprise a cell adhesion material or an endothelialization promoting agent on a surface of a medical device.

In one embodiment, in invention is a drug-releasable medical device characterized by containing the compositions of the present disclosure. The drug-releasable medical device forms a layer of the composition on the surface, and contacts with a living body, or is incorporated or indwelled in a living body. The device includes a stent, a catheter, a clip, an organ replacement medical device, a capsule sensor or an artificial organ. The stent in one embodiment is used for treating coronary artery stenosis and gradually releasing the composition from the surface. The release rate is 1/10³ mu g/mm²/h to 1 mu g/mm²/h on 21 days after indwelling the stent. In addition, the stent of the present invention is characterized in that the drug to be gradually released is carried in a polymeric material coated on the surface of a metal forming the stent or in a porous stent substrate.

The polymeric material coated on the surface of the stent is amorphous. The polymeric material coated on the surface of the stent is an amorphous biodegradable polymeric material. The polymeric material is a poly(lactic acid) or a lactic acid-glycolic acid copolymer, which is biodegradable. The polymeric material further comprises a release auxiliary agent that promotes the release of a drug to be carried. The auxiliary agent that promotes the release of a drug is a tartrate ester or a malate ester, or a monoester or diester of glycerin. The surface of the metal forming the stent may be a porous body and the above-mentioned drug to be gradually released may be carried in the porous body. In one embodiment, the porous body has a pore size of 0.01 nm to 300 nm in diameter.

### Example 5

DEUTERATION of H-COMPOUND to form D-COMPOUND: The H-compound (a compound of the invention, about 1.25 mmol) is dissolved in 3 mL of 100 mM pH=7 deuterated phosphate-buffered saline (D-PBS) diluted with 9.00 mL D₂O to a final concentration of 25 mM. 100 mM D-PBS pH=7 (pH paper) buffer is prepared by dissolving 259.5 mg of K₃PQ₄ in D₂O (12.00 mL) and adding 264 μL 20% DCl in D₂O. The reaction mixture is shaken at room temperature for 11 days while monitoring for completion of hydrogen/deuterium (HID) exchange by LC/MS.

A small scale workup is performed to prepare the hydrochloride salt of the deuterated compound. Thus a 1.2 mL aliquot of the reaction mixture (10% of total volume) is diluted with 5 mL saturated NaHCO₃ and extracted with EtOAc (3×5 mL). The organic layer is dried over Na2S04 and filtered. Evaporation of the solvent gave 20 mg of a colorless oil which is converted to the HCl salt by addition of a few drops of 4M HCl in dioxane. The salt is triturated with ether and the solvents were evaporated to give deuterated compound HCl salt. A 9.6 mL aliquot (80% of total volume) is diluted with 40 mL saturated NaHCO₃ and extracted once with EtOAc (200 mL). The organic layer is quickly dried over Na₂SO₄. Evaporation of the solvent gives the compound which is stored in a freezer.

### Example 6

DEXTROMETHORPHAN MALATE: Dissolve the free base dextromethorphan (0.05 mole) in 20 ml of acetone, add the solution to a solution of the malic acid (0.05 mole) in 60 ml of hot water, and then cool the reaction mixture to separate crystals by filtration and dry.

### Example 7

DEXTROMETHORPHAN METHIONATE: Dissolve the free base dextromethorphan (0.05 mole) in 20 ml of acetone, add the solution to a solution of the methionine or N-acyl methionine (0.05 mole) in 60 ml of hot water, and then cool the reaction mixture to separate crystals by filtration and dry.

### Example 8

DEXTROMETHORPHAN PHTHALLATE: Dissolve the free base dextromethorphan (0.05 mole) in 20 ml of acetone, add the solution to a solution of the phthallic acid (0.05 mole) in 60 ml of hot water, and then cool the reaction mixture to separate crystals by filtration and dry.

### Example 9

DEXTROMETHORPHAN MALONATE: Dissolve the free base dextromethorphan (0.05 mole) in 20 ml of acetone, add the solution to a solution of the malonic acid (0.05 mole) in 60 ml of hot water, and then cool the reaction mixture to separate crystals by filtration and dry.

### Example 10

DEXTROMETHORPHAN TYROSINATE: Dissolve the free base dextromethorphan (0.05 mole) in 20 ml of acetone, add the solution to a solution of the tyrosine or N-acyl tyrosine (0.05 mole) in 60 ml of hot water, and then cool the reaction mixture to separate crystals by filtration and dry.

### Example 11

DEXTROMETHORPHAN TRYPTOPHANATE: Dissolve the free base dextromethorphan (0.05 mole) in 20 ml of acetone, add the solution to a solution of the tryptophan or N-acyl tryptophan (0.05 mole) in 60 ml of hot water, and then cool the reaction mixture to separate crystals by filtration and dry.

### Example 12

DEXTROMETHORPHAN MALEATE: Dissolve the free base dextromethorphan (0.05 mole) in 20 ml of acetone, add the solution to a solution of the maleic acid (0.05 mole) in 60 ml of hot water, and then cool the reaction mixture to separate crystals by filtration and dry.

### Example 13

DEXTROMETHORPHAN SUCCINATE: Dissolve the free base dextromethorphan (0.05 mole) in 20 ml of acetone, add the solution to a solution of the succinic acid (0.05 mole) in 60 ml of hot water, and then cool the reaction mixture to separate crystals by filtration and dry.

### Example 14

DEXTROMETHORPHAN GLUTARATE/GLUTAMAT: Dissolve the free base dextromethorphan (0.05 mole) in 20 ml of acetone, add the solution to a solution of the glutaric acid, glutamic acid or N-acyl glutamic acid (0.05 mole) in 60 ml of hot water, and then cool the reaction mixture to separate crystals by filtration and dry.

### Example 15

DEXTROMETHORPHAN ADIPATE: Dissolve the free base dextromethorphan (0.05 mole) in 20 ml of acetone, add the solution to a solution of the adiptic acid (0.05 mole) in 60 ml of hot water, and then cool the reaction mixture to separate crystals by filtration and dry.

### Example 16

DEXTROMETHORPHAN PIMELATE: Dissolve the free base dextromethorphan (0.05 mole) in 20 ml of acetone, add the solution to a solution of the pimelic acid (0.05 mole) in 60 ml of hot water, and then cool the reaction mixture to separate crystals by filtration and dry.

### Example 17

DEXTROMETHORPHAN SEBACATE: Dissolve the free base dextromethorphan (0.05 mole) in 20 ml of acetone, add the solution to a solution of the sebacic acid (0.05 mole) in 60 ml of hot water, and then cool the reaction mixture to separate crystals by filtration and dry.

### Example 18

DEXTROMETHORPHAN FORMATE: Dissolve the free base dextromethorphan (0.05 mole) in 20 ml of acetone, add the solution to a solution of the formic acid (0.05 mole) in 60 ml of hot water, and then cool the reaction mixture to separate crystals by filtration and dry.

### Example 19

DEXTROMETHORPHAN ACETATE: Dissolve the free base dextromethorphan (0.05 mole) in 20 ml of acetone, add the solution to a solution of the acetic acid (0.05 mole) in 60 ml of hot water, and then cool the reaction mixture to separate crystals by filtration and dry.

### Example 20

DEXTROMETHORPHAN PROPIONATE: Dissolve the free base dextromethorphan (0.05 mole) in 20 ml of acetone, add the solution to a solution of the propionic acid (0.05 mole) in 60 ml of hot water, and then cool the reaction mixture to separate crystals by filtration and dry.

### Example 21

DEXTROMETHORPHAN BUTYRATE: Dissolve the free base dextromethorphan (0.05 mole) in 20 ml of acetone, add the solution to a solution of the butyric acid (0.05 mole) in 60 ml of hot water, and then cool the reaction mixture to separate crystals by filtration and dry.

### Example 22

DEXTROMETHORPHAN VALERATE: Dissolve the free base dextromethorphan (0.05 mole) in 20 ml of acetone, add the solution to a solution of the valeric acid (0.05 mole) in 60 ml of hot water, and then cool the reaction mixture to separate crystals by filtration and dry.

### Example 23

DEXTROMETHORPHAN CAPROATE: Dissolve the free base dextromethorphan (0.05 mole) in 20 ml of acetone, add the solution to a solution of the caproic acid (0.05 mole) in 60 ml of hot water, and then cool the reaction mixture to separate crystals by filtration and dry.

### Example 24

DEXTROMETHORPHAN ENANTHATE: Dissolve the free base dextromethorphan (0.05 mole) in 20 ml of acetone, add the solution to a solution of the enanthoic (heptanoic) acid (0.05 mole) in 60 ml of hot water, and then cool the reaction mixture to separate crystals by filtration and dry.

### Example 25

DEXTROMETHORPHAN CAPRYLATE: Dissolve the free base dextromethorphan (0.05 mole) in 20 ml of acetone, add the solution to a solution of the caprylic acid (0.05 mole) in 60 ml of hot water, and then cool the reaction mixture to separate crystals by filtration and dry.

### Example 26

DEXTROMETHORPHAN PELARGONATE: Dissolve the free base dextromethorphan (0.05 mole) in 20 ml of acetone, add the solution to a solution of the pelargonic acid (0.05 mole) in 60 ml of hot water, and then cool the reaction mixture to separate crystals by filtration and dry.

### Example 27

DEXTROMETHORPHAN CAPRATE: Dissolve the free base dextromethorphan (0.05 mole) in 20 ml of acetone, add the solution to a solution of the capric acid (0.05 mole) in 60 ml of hot water, and then cool the reaction mixture to separate crystals by filtration and dry.

### Example 28

DEXTROMETHORPHAN OXALATE: Dissolve the free base dextromethorphan (0.05 mole) in 20 ml of acetone, add the solution to a solution of the oxalic acid (0.05 mole) in 60 ml of hot water, and then cool the reaction mixture to separate crystals by filtration and dry.

### Example 29

DEXTROMETHORPHAN ISOPHTHALLATE: Dissolve the free base dextromethorphan (0.05 mole) in 20 ml of acetone, add the solution to a solution of the isophthalic acid (0.05 mole) in 60 ml of hot water, and then cool the reaction mixture to separate crystals by filtration and dry.

### Example 30

DEXTROMETHORPHAN TEREPHTHALLATE: Dissolve the free base dextromethorphan (0.05 mole) in 20 ml of acetone, add the solution to a solution of the terephthalic acid (0.05 mole) in 60 ml of hot water, and then cool the reaction mixture to separate crystals by filtration and dry.

### Example 31

DEXTROMETHORPHAN SALICYLATE: Dissolve the free base (0.05 mole) in 20 ml of acetone, add the solution to a solution of the salicylic acid (0.05 mole) in 60 ml of hot water, and then cool the reaction mixture to separate crystals by filtration and dry.

### Example 32

DEXTROMETHORPHAN ACETYLSALICYLATE: Dissolve the free base (0.05 mole) in 20 ml of acetone, add the solution to a solution of the acetyl salicylic acid (0.05 mole) in 60 ml of hot water, and then cool the reaction mixture to separate crystals by filtration and dry.

### Example 33

DIACID ADDITION SALT OF DEXTROMETHORPHAN AND A COMPOUND SELECTED FROM FORMULA I COMPOUNDS: Dissolve the free base (0.25 mole) and dextromethorphan (0.25 mole) in 20 ml of acetone, add the solution to a solution of a di or tri acid (0.05 mole) in 60 ml of hot water, and then cool the reaction mixture to separate crystals by filtration and dry. The di and tri acids include, but not limited to adipic acid, aspartic acid, N-acyl aspartic acid, citric acid, fumaric acid, galactonic acid, glutaric acid, glutamic acid, N-acyl glutamic acid, glucaric acid (saccharic acid), malic acid, maleic acid, mannonic acid, mucic acid, oxalic acid, pimelic acid, phthallic acid, isophthallic acid, terephthallic acid, rhamnonic acid, sebacic acid, succinic acid, and tartaric acid. Thus, forming addition salts such as:

### Example 34

Adipic acid addition salt of dextromethorphan

### Example 35

Aspartic acid addition salt of dextromethorphan

### Example 36

N-Acyl aspartic acid addition salt of dextromethorphan

### Example 37

Citric acid addition salt of dextromethorphan

### Example 38

Fumaric acid addition salt of dextromethorphan

### Example 39

Galactonic acid addition salt of dextromethorphan

### Example 40

Glutaric acid addition salt of dextromethorphan

### Example 41

Glutamic acid addition salt of dextromethorphan

### Example 42

N-Acyl glutamic acid addition salt of dextromethorphan

### Example 43

Glucaric acid (saccharic acid) addition salt of dextromethorphan

### Example 44

Malic acid addition salt of dextromethorphan

### Example 45

Maleic acid addition salt of dextromethorphan

### Example 46

Mannonic acid addition salt of dextromethorphan

### Example 47

Mucic acid addition salt of dextromethorphan

### Example 48

Oxalic acid addition salt of dextromethorphan

### Example 49

Pimelic acid addition salt of dextromethorphan

### Example 50

Phthallic acid addition salt of dextromethorphan

### Example 51

Isophthallic acid addition of dextromethorphan

### Example 52

Terephthallic acid addition salt of dextromethorphan

### Example 53

Rhamnonic acid addition salt of dextromethorphan,

### Example 54

Sebacic acid addition salt of dextromethorphan

### Example 55

Succinic acid addition salt of dextromethorphan

### Example 56

Tartaric acid addition salt of dextromethorphan

### Example 57

Adipic acid addition salt of CBD

### Example 58

Aspartic acid addition salt of CBD

### Example 59

N-Acyl aspartic acid addition salt of CBD

### Example 60

Citric acid addition salt of CBD

### Example 61

Fumaric acid addition salt of CBD

### Example 62

Galactonic acid addition salt of CBD

### Example 63

Glutaric acid addition salt of CBD

### Example 64

Glutamic acid addition salt of CBD

### Example 65

N-Acyl glutamic acid addition salt of CBD

### Example 66

Glucaric acid (saccharic acid) addition salt of CBD

### Example 67

Malic acid addition salt of CBD

### Example 68

Maleic acid addition salt of CBD

### Example 69

Mannonic acid addition salt of CBD

### Example 70

Mucic acid addition salt of CBD

### Example 71

Oxalic acid addition salt of CBD

### Example 72

Pimelic acid addition salt of CBD

### Example 73

Phthallic acid addition salt of CBD

### Example 74

Isophthallic acid addition of CBD

### Example 76

Rhamnonic acid addition salt of CBD

### Example 77

Sebacic acid addition salt of CBD

### Example 78

Succinic acid addition salt of CBD

### Example 79

Tartaric acid addition salt of CBD

General Experimental Details

Anhydrous solvents were purchased from Aldrich Chemical Company (Milwaukee, Wis., USA). Samples of Δ9-THC and Δ8-THC were obtained from RBI/Sigma (Natick, Mass., USA). (+)-p-Menth-2-ene-1,8-diol was prepared as described in a co-pending patent application by the present inventors. TLC plates (silica gel GF, 250 micron, 10×20 cm) were purchased from Analtech (Newark, Del., USA). TLCs were visualized under short wave UV, and then by spraying with ceric ammonium nitrate/sulfuric acid and heating. Column chromatography was carried out using TLC grade silica gel purchased from Aldrich Chemical Company. NMR spectra were obtained on a Bruker 300 MHz instrument. HPLC area percentages reported here are not corrected. HPLCs were run on Shimadzu LC-10AD.

### Example 80

One-Step Reaction of bis(diphenylacetate) Compound with Olivetol (3) to Produce Δ9-THC

Preparation of bis(diphenylacetate)

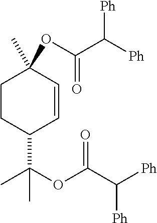 [SMILES: CC(C)(OC(=O)C(c1ccccc1)c1ccccc1)[C@H]1C=C[C@](C)(OC(=O)C(c2ccccc2)c2ccccc2)CC1]

A 25 ml three-necked roundbottom flask with a stir bar was oven-dried, fitted with septa, and cooled under N2. Pyridine (12 ml) was added and the pale yellow solution was stirred. Diphenylacetyl chloride (5.69 g, 4.2 eq.) was added. The solution turned brown. N,N-dimethylaminopyridine (0.1435 g, 0.2 eq.) was added. The mixture was stirred for 1 hour. (+)-p-Menth-2-ene-1,8-diol (1.00 g) was added. The mixture became a lighter colour and solids precipitated. The slurry was allowed to stir overnight at room temperature. The reaction was quenched with water. The mixture was extracted three times with ethyl acetate. The organics were combined and washed with 2M HCl, saturated NaHCO₃, and saturated NaCl (aq.), dried over Na2SO4, filtered and concentrated in vacuo to orange oil. The oil was dissolved in hot methanol and cooled to crystallize. The white solid was collected and washed twice with cold methanol. After drying under vacuum, the yield was 3.282 g (76.8% yield). 1H NMR (CDCl3): δ (ppm) 7.4-7.2 (m, 20H), 5.89-5.84 (dd, 1H), 5.51-5.47 (dd, 1H), 4.90 (s, 2H), 2.7-2.6 (m, 1H), 2.0-1.9 (m, 2H), 1.7-1.6 (m, 1H), 1.43 (s, 3H), 1.42 (s, 3H), 1.40 (s, 3H), 1.35-1.2 (m, 1H). 13C NMR: δ (ppm) 171.47, 171.44, 139.06, 138.84, 132.38, 128.64, 128.56, 128.51, 128.46, 128.28, 127.11, 127.07, 127.02, 85.12, 80.91, 58.32, 57.86, 44.22, 33.81, 25.41, 23.32, 22.81, 21.41. M.p. 111° C. Elemental Analysis: 81.66% C, 6.59% H. Rf (20% EtOAc/hexane): 0.54. [α]D 25=+61.50 (c=1.00, CHCl3). IR (KBr, cm-1): 3061, 3028, 1720.5 (carbonyl stretch).

### Example 81

A 25 ml roundbottom flask with a stir bar was oven-dried, fitted with septa, and cooled under N2. The bis(diphenylacetate) (4) (279 mg, 0.499 mmol) and olivetol (90 mg) were added. Anhydrous CH2Cl2 (8 ml) was added and stirred. The solution was cooled to −5° C. internal temperature. BF3.(OEt)2 (64 μl, 1.0 eq.) was added. The solution gradually darkened to orange. After 30 minutes, the reaction was quenched with 10% Na2CO3 (10 ml). The layers were separated and the organic layer was washed with 2×5 ml 10% Na2CO3. The aqueous washes were combined and extracted twice with CH2Cl2. The organics were combined and washed with water and saturated NaCl solution, then dried over Na2SO4, filtered, and concentrated in vacuo to light yellow oil. The oil was chromatographed on 5 g TLC mesh silica to yield 135.2 mg (86.1%) of Δ9-THC. NMR did show a small amount of solvent present. HPLC showed 96.6 area percent Δ9-THC. 1H NMR agreed with published reports and commercial samples. 13C NMR (CDCl3): δ (ppm) 154.81, 154.16, 142.82, 134.41, 123.74, 110.11, 107.54, 77.18, 45.83, 35.47, 33.58, 31.52, 31.17, 30.63, 27.58, 25.03, 23.34, 22.53, 19.28, 13.99. HPLC R.T.: 28.34 min. Rf (10% MTBE/hexane): 0.30. [α]D 25=−174.2° (c=1.16, EtOH).

### Example 82

Reaction of bis(diphenylacetate) Compound with Olivetol to Produce Ring-Open Intermediate

A 25 ml 2-neck roundbottom flask with a stir bar was oven-dried, fitted with septa, and cooled under N2. Bis(diphenylactetate) (4) (279 mg, 0.499 mmol) and olivetol (90 mg) were added. Anhydrous CH2Cl2 (8 ml) was added. The solution was stirred to dissolve the solids and then cooled to −20° C. internal temperature. BF3.(OEt)2 (16 μl, 0.25 eq.) was added. The solution was stirred for 12 minutes and then quenched with 10% Na2CO3 (aq.) (6 ml). The mixture was extracted twice with CH2Cl2. The combined organics were washed with water and saturated NaCl, dried over Na2SO4, filtered, and concentrated in vacuo to oil. Chromatography on 10 g TLC mesh silica gel (2% MTBE/hexane-15%) yielded A9-THC (fractions 16-22, 31.4 mg, 20.0% yield), but the predominant product was the diphenylacetate triol (the ring open product corresponding to compound D) (fr. 24-37, 160 mg, 60.7% yield). 1H NMR (CDCl3): δ (ppm) 7.26-71.8 (m, 10H), 6.26 (br s, 1H), 6.04 (br s, 1H), 5.35 (s, 1H), 4.51 (s, 1H), 3.92 (br d, 1H), 2.43-2.36 (m, 3H), 2.1-1.9 (m, 2H), 1.79 (m, 1H), 1.71 (s, 3H), 1.6-1.4 (m, 2H), 1.44 (s, 3H), 1.42 (s, 3H), 1.3-1.2 (m, 4H), 0.85 (t, 3H). 13C NMR (CDCl3) 8 ppm 171.56, 142.87, 139.24, 139.08, 128.64, 128.36, 128.31, 126.92, 126.89, 124.93, 115.43, 87.27, 57.53, 45.94, 35.43, 33.46, 31.51, 30.60, 29.96, 24.04, 23.34, 23.20, 23.17, 22.48, 13.97. Rf (20% EtOAc/hexane): 0.48. [α]D 25=−45.9° (c=1.298, CHCl3). Elemental Analysis: 78.69% C, 8.93% H.

### Example 83

One-Step Reaction of Monoacetate with Olivetol to Produce Δ9THC

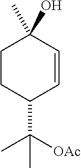

A 25 ml roundbottom flask with a stir bar was oven-dried, fitted with septa, and cooled under N2. The monoacetate (8) (109 mg) and olivetol (92.5 mg) were added. Anhydrous CH2Cl2 (8 ml) was added and stirred. The solution was cooled to −5° C. internal temperature. BF3.(OEt)2 (65 μl, 1.0 eq.) was added. The solution gradually darkened to orange. After 24 minutes, the reaction was quenched with 10% Na2CO3. The layers were separated and the aqueous layer was extracted twice with CH2Cl2. The organics were combined and washed with water and saturated NaCl solution, then dried over Na2SO4, filtered, and concentrated in vacuo to oil. HPLC showed 64.0 area percent Δ9-THC. The oil was chromatographed on 20 g TLC mesh silica to yield 58.7 mg (36.3%) of Δ9-THC. 1H NMR agreed with published reports and commercial samples.

### Example 84

Reaction of Monoacetate Compound (8) with Olivetol to Produce Ring-Open Intermediate

A 25 ml 2-neck roundbottom flask with a stir bar was oven-dried, fitted with septa, and cooled under N2. The monoacetate (8) (109 mg, 0.514 mmol) and olivetol (92.5 mg) were added. Anhydrous CH2Cl2 (8 ml) was added. The solution was stirred to dissolve the solids and then cooled to −20° C. internal temperature. BF3.(OEt)2 (16 μl, 0.25 eq.) was added. The solution was stirred for 45 minutes and then quenched with 10% Na2CO3 (aq.) (4 ml). The mixture was extracted twice with CH2Cl2. The combined organics were washed with water, dried over Na2SO4, filtered, and concentrated in vacuo to a colourless oil. Chromatography on silica gel yielded 90.5 mg (47.0% yield) of acetyl triol (the ring open product corresponding to compound D). 1H NMR (CDCl3): δ (ppm) 6.22 (br m, 2H), 5.76 (br s, 2H), 5.36 (s, 1H), 4.00 (br d, 1H), 2.67 (dt, 1H), 2.40 (t, 2H), 2.26-2.16 (m, 1H), 2.07-1.90 (m, 2H), 1.73 (s, 3H), 1.51 (s, 3H), 1.49 (s, 3H), 1.42 (s, 3H), 1.32-1.24 (m, 41H), 0.85 (t, 3H). 13C NMR (CDCl3): δ (ppm) 170.83, 142.69, 138.03, 124.99, 115.42, 85.90, 44.29, 35.38, 33.47, 31.49, 30.66, 30.09, 25.16, 24.65, 23.17, 22.57, 22.43, 21.84, 13.95. Rf (20% EtOAc/hexane): 0.37.

### Example 85

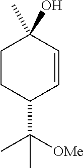

A 25 ml roundbottom flask with a stir bar was oven-dried, fitted with septa, and cooled under N2. The monomethoxy compound (9) (91.9 mg) and olivetol (90 mg) were added. Anhydrous CH2Cl2 (8 ml) was added and stirred. The solution was cooled to −5° C. internal temperature. BF3.(OEt)2 (16 μl, 0.25 eq.) was added. After 1 hour another 16 μl was added. Two hours later, another 32 μl was added. The solution gradually darkened to orange. TLC showed a mixture of Δ9-THC and the ring open product, and major spots. The reaction was quenched with 10% Na2CO3. The layers were separated and the organic was washed with water and sat. NaCl, then dried over Na2SO4, filtered, and concentrated in vacuo to oil.

### Example 86

Reaction of Monomethoxy Compound with Olivetol to Produce Ring-Open Intermediate

A 5 ml roundbottom flask with a stir bar was oven-dried, fitted with a septum, and cooled under N2. The monomethoxy compound (9) (33.5 mg) in 1.5 ml of anhydrous methylene chloride was added. Olivetol (32.7 mg) and magnesium sulfate (134 mg) were added. p-Toluenesulfonic acid monohydrate (34.6 mg) was added. The slurry was stirred at room temperature for 30 minutes. Solid NaHCO₃(100 mg) was added and stirred. The solids were removed by filtration. The solution was washed once with 5% NaHCO₃(aq.). The aqueous wash was extracted once with CH2Cl2. The organics were combined, washed with water, and dried over Na2SO4. The solution was concentrated in vacuo and chromatographed on silica gel. Colourless oil of the methoxy triol (the ring open product corresponding to compound D) (35.3 mg, 56.0% yield) was obtained. 1H NMR (CDCl3): δ (ppm) 7.90 (br s, 1H), 6.68 (br s, 1H), 6.33-6.21 (br d, 2H) 5.75 (s, 1H), 3.74 (s, 1H), 3.20 (s, 3H), 2.44 (t, 2H), 2.07 (br s, 2H), 2.00-1.77 (m, 3H), 1.80 (s, 3H), 1.54 (m, 2H), 1.31 (m, 3H), 1.14 (s, 3H, 1.13 (s, 3H), 0.87 (t, 3H). 13C NMR (CDCl3): δ (ppm) 186.50, 169.63, 166.85, 143, 41, 140.11, 123.58, 79.32, 48.63, 48.05, 35.51, 32.62, 31.52, 30.63, 27.76, 23.74, 23.01, 22.53, 21.95, 20.39, 13.99. Elemental Analysis: 73.3% C, 8.80% H. Rf (10% EtOAc/hexane): 0.25. [α]D 25=−22.70 (c=0.088, CHCl3).

### Example 87

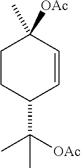 [SMILES: CC(=O)OC(C)(C)[C@H]1C=C[C@](C)(OC(C)=O)CC1]

A 100 ml three-necked roundbottom flask with a stir bar was oven-dried, fitted with septa, and cooled under N2. (+)-p-menth-2-ene-1,8-diol (10.00 g) was added. Triethylamine (68.7 ml, 8.4 eq.) was added and the slurry was stirred. N,N-dimethylaminopyridine (1.435 g, 0.2 eq.) was added. Acetic anhydride (23.3 ml) was placed in an addition funnel and added slowly over 15 minutes. The yellow solution became homogeneous. The solution was warmed to 35° C. internal temperature and stirred for 2.5 hours, then raised to 40° C. for another three hours, then allowed to stir for 13 hours at room temperature. The reaction was quenched with water while cooling in ice. The mixture was extracted three times with hexane and once with ethyl acetate. The organics were combined and washed with saturated NaCl (aq.), dried over Na2SO4, filtered and concentrated in vacuo to an orange oil. Chromatography on 50 g TLC mesh silica yielded the diacetate (10) as a colourless oil (12.3 g, 82.3%). The oil was cooled in dry ice to freeze the oil and then the solid was broken up with a spatula. It was allowed to warm to room temperature and it remained a white solid. 1H NMR (CDCl3): δ (ppm): 5.84 (dd, 1H), 5.54 (dd, 1H), 2.70 (m, 1H), 2.05-1.8 (m, 3H), 1.85 (s, 6H), 1.68 (m, 1H), 1.40 (s, 3H), 1.30 (s, 3H), 1.29 (s, 3H). 13C NMR (CDCl3): δ (ppm) 169.95, 169.89, 132.40, 127.88, 83.79, 79.73, 43.62, 33.85, 25.26, 23.10, 22.74, 22.05, 21.49. m.p. 28-31° C. Elemental Analysis: 65.26% C, 8.61% H. Rf (20% EtOAc/hexane): 0.52. [α]D 25=+73.5° (c=0.99, CHCl3).

### Example 88

A 25 ml roundbottom flask with a stir bar was oven-dried, fitted with septa, and cooled under N2. The diacetate (126.9 mg, 0.499 mmol) and olivetol (90 mg, 0.499 mmol) were added. Anhydrous CH2Cl2 (8 ml) was added and stirred. The solution was cooled to −5° C. internal temperature. BF3.(OEt)2 (64 μl, 1.0 eq.) was added. The solution gradually darkened to red. After 15 minutes, the reaction was quenched with 10% Na2CO3. The layers were separated and the organic layer was washed with 10% Na2CO3. The combined aqueous were extracted once with CH2Cl2. The organics were combined and washed with water and saturated NaCl solution, then dried over Na2SO4, filtered, and concentrated in vacuo to a tannish oil (0.132 mg). HPLC showed 88.8 area percent Δ9-THC. Chromatography on silica gel yielded 95.9 mg (61.0% yield) of Δ9-THC. HPLC showed 94.9 area percent Δ9-THC.

### Example 89

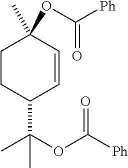 [SMILES: CC(C)(OC(=O)c1ccccc1)[C@H]1C=C[C@](C)(OC(=O)c2ccccc2)CC1]

A 25 ml three-necked roundbottom flask with a stir bar was oven-dried, fitted with septa, and cooled under N2. (+)-p-Menth-2-ene-1,8-diol (1.00 g) was added. Pyridine (6 ml, 12.6 eq.) was added and the pale yellow solution was stirred. N,N-dimethylaminopyridine (0.1435 g, 0.2 eq.) was added. Benzoyl chloride (2.73 ml, 4 eq.) was added. After 10 minutes, a solid precipitated. The slurry was allowed to stir overnight at room temperature. The reaction was quenched with water. The mixture was extracted three times with CH2Cl2. The organics were combined and washed with water and saturated NaCl (aq.), dried over Na2SO4, filtered and concentrated in vacuo. The oil was chromatographed on 25 g TLC mesh silica to yield a colourless oil. The oil was cooled in dry ice and froze, but melted on warming to room temperature. 1H NMR (CDCl3) δ (ppm): 8.0 (dt, 4H), 7.51 (m, 2H), 7.40 (dt, 4H), 6.16 (dd, 1H), 5.88 (dd, 1H), 3.00 (m, 1H), 2.29 (m, 2H), 2.02 (m, 1H), 1.70 (s, 3H), 1.62 (s, 314), 1.60 (s, 3H), 1.25 (m, 1H). 13C NMR (CDCl3) δ (ppm): 165.53, 132.80, 132.53, 132.50, 131.77, 131.63, 129.40, 129.36, 128.39, 128.22, 128.16, 80.64, 44.55, 34.09, 25.81, 23.50, 23.10, 22.59, 21.99, 14.14, 14.05. Elemental Analysis: 76.21% C, 6.97% H. Rf (20% EtOAc/hexane): 0.57.

A 25 ml roundbottom flask with a stir bar was oven-dried, fitted with septa, and cooled under N2. The dibenzoate (189 mg, 0.499 mmol) and olivetol (90 mg) were added. Anhydrous CH2Cl2 (8 ml) was added and stirred. The solution was cooled to −5° C. internal temperature. BF3.(OEt)2 (64 μl, 1.0 eq.) was added. The solution gradually darkened to red. After 15 minutes, the reaction was quenched with 10% Na2CO3. The layers were separated and the organic layer was washed with water and saturated NaCl solution, then dried over Na2SO4, filtered, and concentrated in vacuo to oil. HPLC showed 78.8 area percent Δ9-THC.

### Example 90

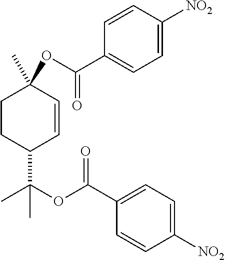 [SMILES: CC(C)(OC(=O)c1ccc([N+](=O)[O-])cc1)[C@H]1C=C[C@](C)(OC(=O)c2ccc([N+](=O)[O-])cc2)CC1]

A 25 ml three-necked roundbottom flask with a stir bar was oven-dried, fitted with septa, and cooled under N2. (+)-p-Menth-2-ene-1,8-diol (1.00 g) was added. Pyridine (6 ml, 12.6 eq.) was added and the pale yellow solution was stirred. N,N-dimethylaminopyridine (0.1435 g, 0.2 eq.) was added. p-Nitrobenzoyl chloride (4.58 ml, 4.2 eq.) was added. After a few minutes, tan solid precipitated. More pyridine (12 ml) was added. The slurry was allowed to stir overnight at room temperature. The reaction was quenched with water. The mixture was extracted three times with ethyl acetate. The organics were combined and washed twice with saturated NaCl (aq.), dried over Na2SO4, filtered and concentrated in vacuo to light yellow solid. The solid was recystallized from isopropyl alcohol and dried under vacuum. The yield was 3.303 g (120% yield), which clearly still contained pyridine and isopropyl alcohol by NMR. It was dried more and then recrystallized from ethyl acetate/hexane to give a lightly coloured solid (1.89 g, 68.7%). 1H NMR (d6-acetone) still seemed to have too many aryl protons. 1H NMR (CD2Cl2) δ (ppm): 8.3-8.2 (m, 4H), 8.2-8.1 (m, 4H), 6.14 (dd, 1H), 5.88 (d, 1H), 3.04 (m, 1H), 2.29 (m, 2H), 2.00 (m, 1H), 1.70 (s, 3H), 1.62 (s, 3H), 1.60 (s, 3H0, 1.67-1.65 (m, 2H). 3C NMR (CD2Cl2) δ (ppm): 164.275, 164.244, 151.00, 133.00, 131.46, 131.09, 131.04, 129.29, 124.00, 123.96, 87.04, 82.75, 45.00, 34.55, 26.10, 23.83, 23.45, 22.64. m.p>200° C. (decomposition). Elemental Analysis: 59.68% C, 4.71% H, 6.07% N. Rf (20% EtOAc/hexane): 0.41. [α]D 25=+38.0° (c=0.21, CHCl3).

A 10 ml roundbottom flask with a stir bar was oven-dried, fitted with septa, and cooled under N2. The di-p-nitrobenzoate (12) (116.5 mg) and olivetol (45 mg) were added. Anhydrous CH2Cl2 (4 ml) was added and stirred. The solution was cooled to −5° C. internal temperature. BF3.(OEt)2 (32 μl, 1.0 eq.) was added. The cloudy solution gradually darkened to orange. After 2 hours, the reaction was quenched with 10% Na2CO3. The layers were separated and the organic layer was washed with water and sat. NaCl, then dried over Na2SO4, filtered, and concentrated in vacuo to yellow oil. HPLC showed 71.5 area percent Δ9-THC.

### Example 91

Preparation of Cannabidiol By Condensation of Olivetol With Menthadienol

A solution of olivetol (1 g, 5.56 mmol) in dichloromethane (15 mL) was treated with Zn(OTf)2 (10 mg, 0.5 mol %) and heated to 60° C. in a modified pressure tube equipped with a septum for additions. A solution of menthadienol (0.63 g, 0.75 equiv, 4.14 mmol) in dichloromethane (5 mL) was added via syringe over 5.5 h. HPLC analysis of the reaction after a total of 6 h showed a 2.6:1 ratio of cannabidiol to abn-cannabidiol (47.0%:17.9%) with 23.3% unreacted olivetol and 4.3% of a double addition product. Only trace levels of cyclized degradants (delta-8- and delta-9-THC) were observed, even after continuation of the heating for a total of 20 h.

### Example 92

To a stirred mixture of potassium carbonate (2.98 kg) in ethanol (16.7 L) was added (+)-limonene oxide (25.0 kg) and the mixture heated to 60° C. Thiophenol (8.86 kg) was added over 11 hours at 70-80° C. Ethanol was distilled out at atmospheric pressure over the course of four hours until the pot temperature reached 105° C., the batch cooled to 80° C. then cold water (16 L) added. After cooling to 40° C., methyl t-butyl ether (MTBE, 16 L) was added. The organic phase was separated, washed with water (4.5 L), and the solvents removed under reduced pressure at 60° C. The residual oil (30.9 kg) was fed to a 4 inch, wiped-film evaporator at 3 mm and 145° C. to remove the unreacted limonene oxide. The nonvolatile fraction (14.7 kg of thiophenyl ether) was dissolved in glacial acetic acid (26.0 L) and stirred while 35% hydrogen peroxide (6.0 kg) added over 6.5 hours. The reaction temperature was maintained at 10-20° C. The reaction was allowed to warm to room temperature overnight, then transferred into a mixture of warm water (89 L, 40-45° C.) and MTBE (34 L). The organic phase was washed aqueous 5% sodium bicarbonate (4 washes, 18 L each) at 40-45° C., to achieve a final pH of ca. 8 and a negative starch-iodine test. The organic phase was concentrated under reduced pressure at 60° C. to obtain a residue of crude sulfoxide mixture (14.8 kg, estimated 95% yield). This residual was dissolved in tetraglyme (11.6 L) and stored until needed, during this time the product partially crystallized. The solution was gently warmed to redissolve the sulfoxide mixture. To a portion of this tetraglyme solution (containing ca. 8.3 kg of sulfoxide mixture) was added potassium carbonate (2.7 kg) and tetraglyme (5 L) and the stirred mixture was heated to 180° C. with application of vacuum (75-80 mm), distilling the volatiles over the course of nine hours. The strong smelling distillate (ca. 2.8 kg) was dissolved in heptane (2.5 L) and washed with water (4.5 L+1 L), then concentrated under reduced pressure at 60° C. to crude (+)-menthadienol (1.76 kg) of approximately 83% purity by GC. Several batches of crude (+)-menthadienol (totaling ca. 6.68 kg) were combined with hexadecane (1.00 kg) and solid potassium carbonate (67 grams) in a stirred round bottom flask fitted with a 5 plate 2 inch diameter Oldershaw column fitted with a reflux return splitter. Distillation was effected at pot temperature of ca. 105-110° C. with vacuum of ca. 1-5 mm. After the initial low boiling fractions were removed (bp: 45-75° C.), main fractions of boiling point: 75-80° C. were collected totaling 4.0 kg of (+)-menthadienol (assayed at 95-98% by gas chromatography (AUC)). Optical rotation of a sample prepared by this procedure was +75.4° (c=1.074 in chloroform at 25° C.). The literature value is +67.9° at 20° C. (Ohloff et al., Helvetica Chimica Acta, 63:76 (1980), which is hereby incorporated by reference in its entirety).

### Example 93

To a stirred mixture of anhydrous ethanol (10.5 L) and diethylmalonate (1.90 kg) at 20° C. was added, over 35 minutes, a sodium ethoxide solution (21% in ethanol, 4.2 L). The reaction temperature was allowed to rise to 27° C. To the resulting slurry was added 3-nonene-2-one (1.50 kg), over the course of three hours, allowing the temperature to rise to 45-50° C. The reaction mixture heated to 70° C. over two hours and held for an additional two hours. The reaction mixture was then cooled to 0° C. and held overnight. The solid product was then collected by filtration through a polypropylene filter. The solid cake was rinsed with MTBE (5.0 L) then dried under reduced pressure at 20-25° C. to constant weight affording 2.38 kg (99% yield) of sodium ethyl dihydroolivetolate as an off-white solid. 1H NMR 500 MHz (DMSO-d6) δ 0.85 (t, 3H), 1.1-1.5 (m, 11H), 1.7 (dd, 1H), 2.05, dd, 1H), 2.4 (m, 1H), 2.7 (d, 1H), 4.05 (q, 2H) and 4.4 ppm (s, 1H). HPLC analysis showed 100% product (Phenomenex (Houston, Tex.) HyperClone 5u BDS C18 column, 4.6×150 mm, 1 mL/min, gradient 100% water/0.1% TFA to 100% acetonitrile/0.1% TFA over 15 minutes, rt=8.0 min).

### Example 94

To a stirred suspension of sodium ethyl dihydroolivetolate (200.8 g, 0.727 mol) and anhydrous sodium acetate (238.5 g, 2.91 mol) in acetic acid (1010 mL) at 50° C. was dropwise added bromine (655.5 g, 2.29 mol) over the course of three hours while maintaining the batch temperature at 50-55° C. After stirring an additional hour at 50-55° C., the slurry was cooled over three hours to 20° C. Water (925 mL) was added over 1 h during which the product crystallized. The slurry was cooled to 10° C., held overnight, and then filtered through filter paper. The solid cake was washed with water (3×400 mL, to achieve a final rinse pH of 4) and then air dried overnight to obtain 310 g (86% yield) of crude ethyl dibromoolivetolate, containing ca. 11.7% by weight of water. 1H NMR, 500 MHz (CDCl3) δ 0.9 (t, 3H), 1.4 (m, 8H), 1.6 (t, 3H), 3.1 (m, 2H), 4.4 (m, 2H), 6.4 (s, 1H) and 12.3 ppm s, 1H). HPLC analysis showed 98.5% product (AUC, Sunfire reversed phase C18 column from Waters Corporation (Milford, Mass.), 4.6×150 mm, 1 mL/min, gradient 80% 0.1% TFA in water with 20% 0.5% TFA in acetonitrile to 100% of 0.5% TFA in acetonitrile over 15 minutes, rt=13.8 min).

### Example 95

A 2 L Parr reactor charged with ethyl dibromoolivetolate (160.3 g of water wet material, 0.345 mol), ethanol (290 mL), water (440 mL), sodium citrate (220 g, 0.747 mol) and 5% palladium-on-charcoal catalyst (7.4 g) was degassed with nitrogen and then pressurized to 50 psig with hydrogen gas. The stirrer was started and the reaction mixture was heated to 60° C. and maintained at that pressure and temperature for six hours after which the heat was turned off. After cooling to ambient temperature, the mixture was filtered through celite (100 g) and the reactor and solid filter cake was rinsed with water (600 mL), then toluene (300 mL). The layers were separated and the organic phase was evaporated under reduced pressure to a semisolid residue. Heptane (260 mL) was added and the mixture was warmed to 45° C. at which point the solids dissolved. The stirred mixture was allowed to cool slowly to ambient temperature overnight, during which crystallization occurred. The slurry was cooled to 5° C., held 4 hours, and the solid product was collected by filtration. The filter cake was rinsed with cold heptane (150 mL) then dried to a constant weight under reduced pressure at 20° C. to afford 63.0 g (72% yield) of yellow crystals of ethyl olivetolate. HPLC analysis indicated that the product was 99.6% pure (AUC, Sunfire reversed phase C18 column, 4.6×150 mm, 1 mL/min flow rate, gradient 80% 0.1% TFA in water with 20% 0.5% TFA in acetonitrile to 100% of 0.5% TFA in acetonitrile over 15 minutes, rt=10.3 min). This product was identified by melting point (mp: 68° C.) and NMR analysis. 1H NMR, 500 MHz (CDCl3) δ 0.9 (t, 3H), 1.4 (m, 8H), 1.6 (t, 3H), 2.8 (m, 2H), 4.4 (m, 2H), 5.4 (br s, 1H), 6.2 (s, 1H), 6.3 (s, 1H) and 11.8 ppm (s, 1H).

### Example 96

To a stirred solution of ethyl olivetolate (40.1 g, 155 mmol) in dichloromethane (360 mL) was added anhydrous magnesium sulfate (10.4 g) and scandium triflate (3.93 g, 8 mmol). The mixture was cooled to 10° C. To this slurry was added a cold solution of (+)-menthadienol (25.1 g, 155 mmol) in dichloromethane (160 mL) over three minutes, followed by a dichloromethane rinse (120 mL). A slight exotherm was observed. After stirring at 10° C. for three hours, HPLC analysis showed the reaction was complete by no further decrease in the olivetolate ester concentration. The reaction was quenched by addition of solid anhydrous sodium carbonate (4.0 g) and stirred overnight at 25° C. The reaction mixture was clarified by filtration through a bed of celite and the flask and filter cake was washed with dichloromethane (250 mL). The combined filtrates were concentrated under reduced pressure to about 150 mL of volume. Heptane (400 mL) was added and the mixture was again concentrated under reduced pressure to about 150 mL. Heptane (400 mL) was added and the mixture extracted with aqueous sodium hydroxide solution (2×200 mL of a 20% aqueous solution) followed by water (2×200 mL). HPLC analysis showed the organic phase to be free of any residual ethyl olivetolate. The heptane phase was concentrated under reduced pressure to 58.6 g (87% yield after correcting for the HPLC purity of 90%) of a dark colored oil, primarily ethyl cannabidiolate as determined by HPLC analysis. This crude material is used directly in the next step.

### Example 97

Crude ethyl cannabidiolate (58.6 g, ca. 90% pure by HPLC) was dissolved in methanol (390 mL) and the stirred solution was degassed by refluxing under nitrogen for 1 hour. Aqueous sodium hydroxide solution (80.8 g of NaOH in 390 mL of water) was degassed by refluxing under nitrogen for one hour. The hydroxide solution was transferred under nitrogen pressure, through a steel cannula to the hot ethyl cannabidiolate/methanol solution over 20 minutes while maintaining the reaction at 70° C. After five hours at 70-80° C., the hydrolysis was found complete by HPLC analysis and the reaction cooled to 20° C. The reaction was quenched by addition of a degassed aqueous solution of citric acid (50 wt % solution, 400 g). The mixture was extracted with heptane (400 mL) and the organic layer was washed with aqueous sodium bicarbonate solution (300 mL) and water (300 mL). The heptane solution was concentrated under reduced pressure to ca. 100 mL, reconstituted with heptane (400 mL), concentrated again to ca. 50 mL and heptane (200 mL) was added. The slowly stirred heptane solution was cooled to 10° C., seeded with cannabidiol crystals and stirred slowly at 10° C. for three hours to develop a crop of crystals. The slurry was stored overnight at −5° C. The solid product was collected by filtration on cold sintered glass and the reactor and cake rinsed with cold heptane (150 mL). The solids were dried under nitrogen stream for two hours then under reduced pressure at 20° C. for 15 hours to afford 21 g (44% yield) of solid cannabidiol. HPLC analysis showed 99.6% (AUC) product (Sunfire C18 5u column, 4.6 mm×150 mm, 1 ml/min flow rate, gradient 80% of 0.1% TFA/water and 20% 0.05% TFA/acetonitrile to 100% 0.05% TFA/acetonitrile over 15 minutes, rt=11.9 min).

### Example 98

Ethyl olivetolate (25 g, 99 mmol) was dissolved in dichloromethane (250 mL) and MgSO4 (25 g, 1 wt) and Sc(OTf)3 (4.88 g, 9.9 mmol, 10 mol %) were added sequentially. A solution of menthadienol (24.5 g, 161 mmol, 1.6 equiv of purity of 100% but really approximately 80-85% AUC by GC) in dichloromethane (125 mL) was added over 1.5 h using a dropping funnel. The reaction was monitored by HPLC analysis and after approximately 3 h the reaction was filtered through celite, the solids washed with dichloromethane (125 mL), and the combined organics were evaporated under reduced pressure. The residue was dissolved in heptane and applied to 5 wt silica, then eluted with heptane (1×500 mL), 10% dichloromethane/heptane (6×500 mL), 15% dichloromethane/heptane (2×500 mL) and 20% dichloromethane/heptane (2×500 mL). Fractions containing ethyl cannabidiolate were combined and concentrated to give the product (31.3 g, 82% yield, with a purity of 93.3% AUC by HPLC).

### Example 99

The ethyl cannabidiolate (31.3 g, 81 mmol) was dissolved in MeOH (10 vol, 310 mL) and degassed with argon. Separately, a solution of NaOH (64.8 g, 1.62 mol, 20 equiv) in deionized water (10 vol, 310 mL) was prepared and degassed with argon. The organic solution was added to the aqueous solution under a strict argon atmosphere, then the mixture heated to reflux and held there for 3.5 h, then cooled to room temperature. HPLC analysis indicated completion of reaction. The reaction mixture was quenched with aqueous citric acid (129.6 g citric acid, 8.3 equiv, as a 30% solution in water). The addition was exothermic. Heptane (310 mL, 10 vol) was added to the mixture and the product extracted into the heptane phase. A second extraction using heptane (150 mL, ca. 5 vol) was then performed and HPLC analysis of the aqueous fractions indicated the absence of the cannabidiol. The combined organics were dried by azeotropic distillation of the water and concentrated to ca. 250 mL and then cooled to −16 to −17° C., and seeded with solid cannabidiol when the temperature reached −1.5° C. After 20 h, the resulting solids were filtered off, washed with cold heptane and dried on the filter, then under high vacuum. This ultimately gave 17.9 g cannabidiol (57.5% yield over two steps from ethyl olivetolate) with a purity of >99.8% AUC by HPLC.

### Biological Studies

### Example 100

ANTI-AGITATION AND ANXIOLYTIC EFFECTS OF THE COMPOUND OF FORMULA I GIVEN IN COMBINATION WITH AN NMDA RECEPTOR CHANNEL BLOCKER: A total of 24 seven-month-old male Wistar rats (RccHan: WIST, Envigo, Netherlands) were used. Animals were housed in groups in standard rat cages under a 12/12 h artificial light/dark cycle (lights on at 7:00 a.m.). Room temperature was kept constant (temperature: 22±1° C.). Standard laboratory rat food (4RF21-GLP, Mucedola srl, Milan, Italy) and tap water were provided ad libitum throughout the experimental period. All experimental procedures were carried out in accordance with the Animal Welfare Act and the European Communities Council Directive of 22 Sep. 2010 (2010/63/EU). To measure locomotor activity and exploratory behavior of rats, a dark plexiglas box of four equal squares was used. The light was adjusted to 80 lux (measured in the center of the OF, in the corners light was 50-60 lx). For each test, there was a novel object placed into one corner of the field. Digital camera was located above the apparatus to record movements of rats. Dextromethorphan HBr monohydrate (DXM, Lot No. SLBZ4801, Sigma-Aldrich, Germany) was dissolved in deionized water (vehicle1) and administered orally (administration volume was 4 ml/kg). Cannabidiol (CBD, Batch: CBDAPI2003, THC Pharm, Germany) was dissolved in tween80 and then diluted with saline to a final concentration of 20% (vehicle2) and injected intraperitoneally (i.p.) in a volume of 2 ml/kg. Control experiments were performed following administration of respective vehicle/s. All solutions were freshly prepared. All animals were handled for approx. one week before the experimental procedures and were habituated to the experimental room for approx. 1 hour one day before the experimental procedure. Following placement of the animals in the center of the box, the experimenter left the room while a camera above recorded movements of rats. Each test lasted for 60 min. Apparatus was thoroughly cleaned with 30% ethanol. For the testing of drug effects, rats were administered with DXM (or vehicle) and CBD (or vehicle). Tests were repeated using a Latin square design until a total of n=8 animals were tested in each treatment condition.

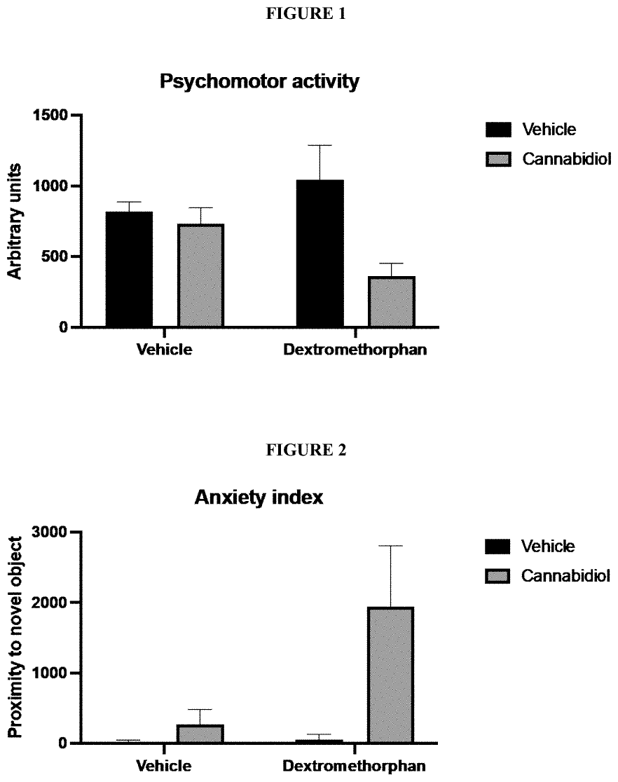

Combined administration of CBD (20 mg/kg) and DXM (60 mg/kg) has significantly reduced motor agitation displayed by the rats under the conditions of moderate bright light in the open field (FIG. 1). Of note, such effects observed when rats were pre-treated with the combination but not by either CBD or DXM given alone, as confirmed by the ANOVA results (F(1,28)=4.2, P<0.05). Further, rats treated with the combination of CBD and DXM have spent more time in the close proximity to the novel object (FIG. 2). The anxiety index was calculated as the increase in the time spent in the quadrant adjacent to the novel object relative to the next corner quadrant (i.e. farther away from the object). ANOVA has confirmed a significant CBD by DXM treatment interaction confirming that the anxiety index was affected by the combination of CBD and DXM and not by either of the drugs alone (F(1,27)=4.4, P<0.05). Taken together, these results suggest the combination of CBD and DXM, but not CBD or DXM alone, can reduce agitation and anxiety.

### Example 101

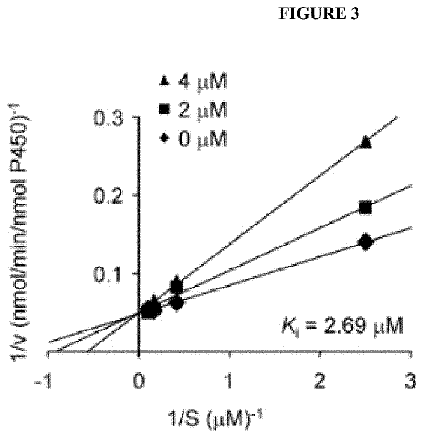

INHIBITION OF CYP 2D6: The activity of dextromethorphan O-demethylation was measured using recombinant CYP2D6 (0.25 pmol). An incubation mixture consisted of an enzyme source, dextromethorphan, an NADPH-generating system (500 μM NADP, 10 mM glucose 6-phosphate, 10 mM magnesium chloride, and 1 unit/ml glucose 6-phosphate dehydrogenase), and 100 mM potassium phosphate buffer (pH 7.4) in a final volume of 200 μl. Incubations were performed at 37° C. for 10 min and were terminated by adding 10 μl of 70% (w/v) perchloric acid. After removal of protein by centrifugation, 50 μl of the supernatant was subjected to a high-performance liquid chromatography (D7500 integrator, L-7100 pump, L-7200 autosampler, L-7300 column oven, and L-7485 fluorescence detector) equipped with a Mightysil RP-18 GP column (4.6μ 250 mm, 5 μm). The mobile phase was the mixture of acetonitrile/methanol: 10 mM potassium phosphate buffer adjusted to pH 3.5 with phosphoric acid (200:160:630). Elution was performed at a flow rate of 1.0 ml/min. The formation of dextrorphan was monitored at an excitation of 280 nm and an emission of 310 nm. As shown in FIG. 3, cannabidiol inhibited metabolism of dextromethorphan with a Ki of 2.69 uM.

What is claimed is:

1. A composition comprising:
a) a compound of Formula I: 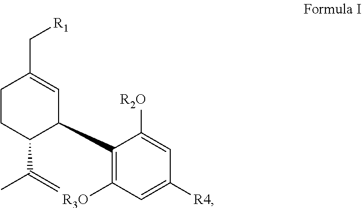
wherein R₁, R₂, R₃, and R₄ are independently H, OH, or Alkyl (C₁-C₁₀), or enantiomers thereof, metabolites thereof, derivatives thereof, and/or prodrugs thereof, pharmaceutically acceptable salts thereof, N-oxides thereof, or a combination thereof, or
b) an NMDA receptor antagonist or a compound of Formula II, 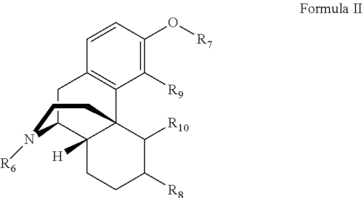
wherein, R6, R1, and Rs are independently H, D, C1-10-alkyl, halo C1-10-alkyl wherein halogen is F, Cl, or Br; R9 and R10 are independently H; C1-10-alkyl; halo C1-10-alkyl wherein halogen is F, Cl, or Br; OH; or R9 and R10 together form a five-membered heterocycle wherein the hetero atom is O, S, or N; enantiomers, metabolites, derivatives, prodrugs, salts, diastereomers, pharmaceutical acceptable salts, or N-oxides thereof, or a combination thereof, or enantiomers thereof, metabolites thereof, derivatives thereof, prodrugs thereof, pharmaceutically acceptable salts thereof, N-oxides thereof, or acid addition salts, or a combination thereof, or
c) a combination of at least one compound of formula I and at least one compound of formula II.

2. The composition of claim 1, wherein the compound of Formula I is 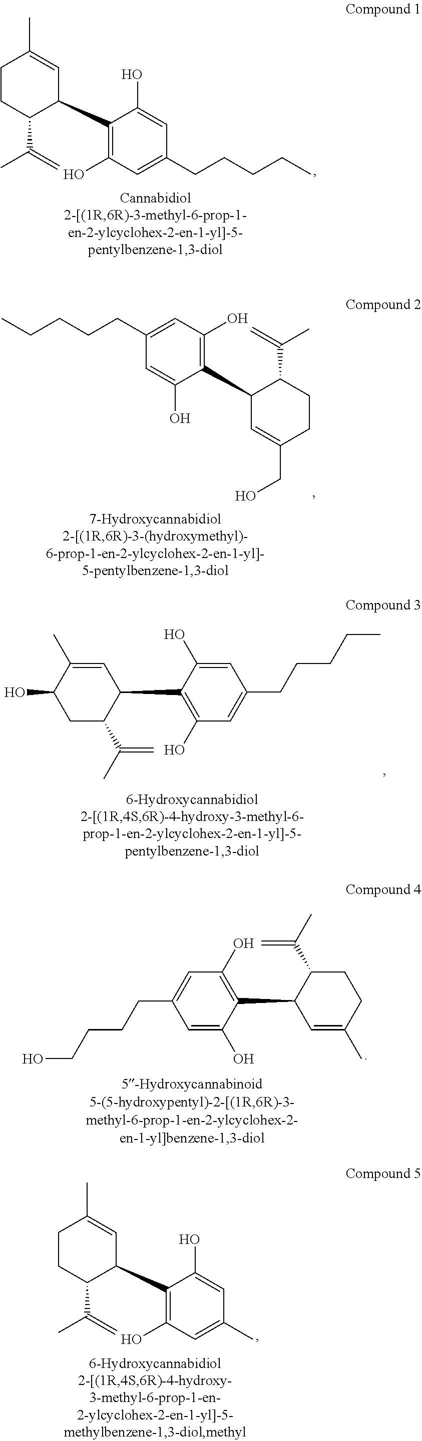 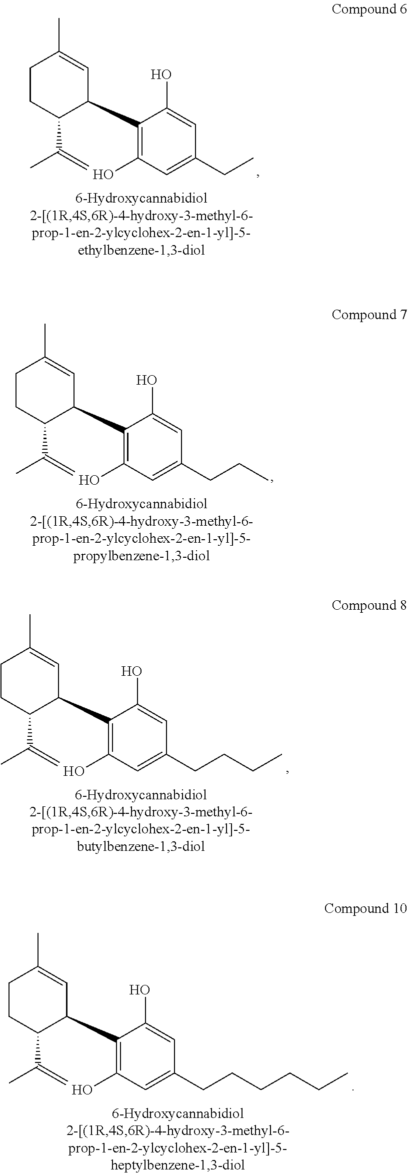 [SMILES: C=C(C)[C@@H]1CCC(C)=C[C@H]1c1c(O)cc(CC)cc1O ; C=C(C)[C@@H]1CCC(C)=C[C@H]1c1c(O)cc(CCC)cc1O ; C=C(C)[C@@H]1CCC(C)=C[C@H]1c1c(O)cc(CCCC)cc1O ; C=C(C)[C@@H]1CCC(C)=C[C@H]1c1c(O)cc(CCCCCC)cc1O]
or a metabolite thereof, derivative thereof, prodrug thereof, pharmaceutically acceptable salt thereof, N-oxide thereof, or acid addition salt; or a combination thereof.

3. The composition of claim 2, wherein the NMDA receptor antagonist selected from the group consisting of: ketamine; methadone; memantine; amantadine;
dextropropoxyphene; ketobemidone; dextromethorphan; (4bS,8aS,9S)-1 1-methyl-3-(trifluoromethoxy)-6,7,8,8a,9, 10-hexahydro-5H-9,4b-(epiminoethano)phenanthrene;
(4bS,8aS,9S)-3-(trifluoromethoxy)-1 1-(trifluoromethyl)-6,7,8,8a,9, 10-hexahydro-5H-9,4b-(epiminoethano)phenanthrene; and
(4bS,8aS,9S)-3-methoxy-1 1-(trifluoromethyl)-6,7,8,8a,9, 10-hexahydro-5H-9,4b-(epiminoethano) phenanthrene;
or an acid addition salt thereof selected from acetate, acetyl salicylate, adipate, aspartate, butyrate, caprate, caproate, caprylate, enanthate, formate, fumarate, glutamate glutarate, hydrobromide, hydrochloride, isophthallate, maleate, malonate, methionate, oxalate, pelargonate, pimelate, propionate, phthallate, salicylate, sebacate, succinate, terephthallate, tyrosinate, tryptophanate, valerate, N-acyl-aspartate, N-acyl-glutamate, N-acyl-tyrosinate, N-acyl-tryptophanate, N-acyl-methionate, citrate, galactonate, glucaric acid (saccharic acid), mannonate, mucate, rhamnonate, and tartrate; or a combination thereof.

4. The pharmaceutical composition of claim 2, wherein the compound of Formula II is dextromethorphan.

5. The pharmaceutical composition of claim 4, wherein the composition further comprises: a. polymer, b. emulsifier, c. binder, d. a disintegrating agent, and/or e. a lubricant.

6. The composition of claim 4, further comprising ajmaline, amiodarone, amitriptyline, amoxapine, aprindine, azelastine, amphetamine, aryloxyindanamine, benactyzine, brasofensine, bupropion, butriptyline, celecoxib, 2-chloroimipramine, chlorpheniramine, chlorpromazine, cimetidine, cisapride, citalopram, clomipramine, clozapine, cocaine, dapoxetine, desipramine, desvenlafaxine, dibenzepin, diphenhydramine, donepezil, dosulepin, doxorubicin, duloxetine, escitalopram, fluoxetine, fluphenazine, fluvastatin, fluvoxamine, galantamine, haloperidol, 1m1pramme, indinavir, iprindole, iproclozide, iproniazid, isocarboxazid, lansoprazole, levomepromazine, lofepramine, lopinavir, loratadine, lurasidone, maprotiline, mequitazine, methadone, methylphenidate, metoclopramide, mianserin, mibefradil, milnacipran, mirtazapine, moclobemide, modafinil, nefazodone, nelfinavir, nevuapme, nialamide, nicardipine, norfluoxetine, nortriptyline, opipramol, perphenazine, phenelzine, pimozide, protriptyline, quinidine, rasagiline, risperidone, ritonavir, rivastigmine, saquinavir, selegiline, sertindole, sertraline, sibutramine, tacrine, terbinafine, terfenadine, tesofensine, thioridazine, ticlopidine, toloxatone, tranylcypromine, trazodone, trifluperidol, trimipramine, venlafaxine, yohimbine, or zuclopenthixol; or a combination thereof.

7. A method of increasing dextromethorphan plasma levels in a subject in need thereof of, wherein the subject is an extensive metabolizer of dextromethorphan, the method comprising administering a therapeutically effective composition of claim 4.

8. The method of claim 7, wherein the composition is administered once or twice a day, wherein the daily dose of dextromethorphan is about 0.1 mg to about 1000 mg, resulting in an AUCo-12 of dextromethorphan that is greater than the AUCo-12 of dextromethorphan that would be achieved by administering the same amount of dextromethorphan without a compound of Formula I.

9. The method of claim 8, wherein the AUCo-12 of a compound of Formula I is at least about 10 ng/hr/mL, about 100 ng/hr/mL, 200 ng/hr/mL, about 300 ng/hr/mL, or about 400 ng/hr/ml, or about 500 ng/hr/mL, or about 600 ng/hr/mL, or about 700 ng/hr/mL, or about 800 ng/hr/mL, or about 900 ng/hr/mL, or about 1000 ng/hr/mL

10. The method of claim 9, wherein the administration is cutaneous, oral, nasal, anal, rectal, vaginal, sublingual, buccal, sublabial, muscular, intramuscular, intravenous, peritoneal, epidural, intracerebral, intracerebroventricular, epicutaneous or topical, intraarticular, intracardiac, intracavernous, intradermal, intralesional, intramuscular, intraocular, intraosseous, intraperitoneal, intrathecal, intrauterine, intravaginal, intravesical, intravitreal, transdermal, or transmucosal.

11. A method of treatment of a subject in need thereof of, comprising:
a) administering a therapeutically effective amount of the composition of claim 4;
b) treating a neuropsychiatric or neurodegenerative disease or disorder, or brain injury, comprising behavioral and psychological symptoms of dementia (BPSD), in a patient in need thereof, and
c) producing a symptomatic relief and/or disease modification.
